SEQUENCE LISTING (19 sequences)

SEQ ID NO: 1 - protein, 873 aa, Infectious laryngotracheitis virus
  REGION 1..873: note = misc_feature - amino acid sequence of the envelope glycoprotein forming spikes at the surface of the virion envelope of ILTV (Gallid herpesvirus 1) strain 632
MASLKMLICVCVAILIPSTLSQDSHGIAGIIDPRDTASMDVGKISFSEAIGSGAPKEPQIRNRIFACSSPTGASVARLAQPRHCHRHADSTNMTEGIAVVFKQNIAPYVFNVTLYYKHITTVTTWALFSRPQITNEYVTRVPIDYHEIVRIDRSGECSSKATYHKNFMFFEAYDNDEAEKKLPLVPSLLRSTVSKAFHTTNFTKRHQTLGYRTSTSVDCVVEYLQARSVYPYDYFGMATGDTVEISPFYTKNTTGPRRHSVYRDYRFLEIANYQVRDLETGQIRPPKKRNFLTDEQFTIGWDAMEEKESVCTLSKWIEVPEAVRVSYKNSYHFSLKDMTMTFSSGKQPFNISRLHLAECVPTIATEAIDGIFARKYSSTHVRSGDIEYYLGSGGFLIAFQKLMSHGLAEMYLEEAQRQNHLPRGRERRQAAGRRTASLQSGPQGDRITTHSSATFAMLQFAYDKIQAHVNELIGNLLEAWCELQNRQLIVWHEMKKLNPNSLMTSLFGQPVSARLLGDIVAVSKCIEIPIENIRMQDSMRMPGDPTMCYTRPVLIFRYSSSPESQFSANSTENHNLDILGQLGEHNEILQGRNLIEPCMINHRRYFLLGENYLLYEDYTFVRQVNASEIEEVSIFINLNATILEDLDFVPVEVYTREELRDTGTLNYDDVVRYQNIYNKRFRDIDTVIRGDRGDAIFRAIADFFGNTLGEVGKALGTVVMTAAAAVISTVSGIASFLSNPFAALGIGIAVVVSIILGLLAFKYVMNLKSNPVQVLFPGAVPPAGTPPRPSRRYYKDEEEVEEDSDEDDRILATRVLKGLELLHKDEQKARRQKARFSAFAKNMRNLFRRKPRTKEDDYPLLEYPSWAEESEDE

SEQ ID NO: 2 - DNA, 3312 bp, Gallid alphaherpesvirus 1
  misc_feature 1..3312: note = wild type polymerase gene (UL30)
  source 1..3312: mol_type = genomic DNA
atggacactttttcctccgccggtactggagctgctcgtgtagccccgataattggcctcgggccgcactctggcgcgtcttattatacttctgtgagagaattcccgtatgtatgtccaacgtgcattaatgggggcggaaggataggaacgcagattggaagggctacaagcaaaccaaccttttaccacaacgagcgacaattagacatcttgacaagcacacatggagcgtggcccgtcaggatgaaatattggaatgctggtcctggaatgactcaccacaacaagggagatgtaaagttttatgaatttcacgtttatgacctttttgaaaacactgagatcgctcagtcatgtgtacgatggatgcattccagatttcttgatacactgaaaccgaccggtactgtaataaccttagttggaaattctgcatgcggaaagcgcgtcgccgttcatgtgtatggatctttgccatacttttttgttaaaaagcgagaaattgaccaagccgcgaaagtcacgaactcggaagagttggcgcatgctctagcattagcgacgcgtaagacatctttaaaaaattcaccttttctagctgtttcagcggaaagtttttgcattgatgttgttcggcgcagagacatttattattctgaatctgaggaggaggacttctaccgtgtaaaagtgtgtaatggcaaagtgatgaaatttatatgtgataattttttcccgagcgtacctaaatatgaaagcaatgttgacgctattaccaggttcatcctcgacaataatctgacttcttttgggtggtatcgctttagagctcaaggtggcgcactccaaattcgagacccaggacaacatgctacatcagctgatgtcgaaattaactgcacagcaagtaatttggaactcggaaacaatgtctcgtggccagactacaaattactgtgctttgatatagaatgtaaagccggaggggcaaatgagtttgcatttccttctgctgagaaggtagaagatcttgttatccaaatttcggcagttacatattctttgttaactaaggaaaaagagcaggaaatcattttttccctagggacctgtgaactttccgaagattgtagtgatgggataactgtatgcaaatgtggctctgagttcgagcttcttctgtgctttatgacttttttcaagcaatattcacccgaatttgtgagcggatacaatattctagggttcgattgggcctatctttctaacaagctgagcaaggtgtatggcatgcgtctcgatggatatgggaaagcaaactcgtgggggacattcaaacttcaagatccgtctgcgcgcggacttggaagattcaaaaaagtcaaaatcaatggcgtcgtcaacattgatatgtttaccattagttatgaaaagctaaagttaccatcttataagttaaatgcagtagcagagtgcgttcttggagaacaaaaaattgatctggcttacaaagatatcccagtcatgtttgctgcaggccctaaagaaagggggaaaataggggagtattgtttgcaggattctagactgtcagggagtttgtttttcaaattctcccctcatttagaaatgtccgctgtcgctaaattggcctgtatacccttgacaagagcaataggcgatggacagcagctgcgcgtttatacatgcttgctccaacgctctacggcgtccgggtttgtgttgccggacaaaaagagtgcgttttccttcggttctactcttgcttcagacgcgtctgatgcacctgctacaagaaatgtaggctaccagggagccaaggtactagatccagaaataggattccatgtaactcctgtggttgtattcgattttgccagtttgtatccgagtattattcagagtaacaatctatgttacagtacgttaacgcacgaccccgccgcactggcaggtctacagccagaaaaagatttcttccagattgatgttcagggtcggaaattttattttgtcagggagcatattcgcaaaagtttgctgtcagacttgctaggtgattggttatctatgaggaaagccgtgcgcgcaaaaatcaagacagccgagactgaagaagagcgaatcttattagacaaacagcaagctgccttaagttgtctggaactctgctatgggtttactggggtgatgcatggaatgcttccttgtttagaagttgcttcaacagttacagctataggaagggacatgcttttgcgtacaaaggcgcacatcgagaaggagtggagaagtggaaatcaatttgccgaaaaatttttgcccgggtccgaacgtattcagctaaaccaatactctgtccgggtcattaatggagaaactgattctgcatattgtgaagtttaccggagtttacatttaaactttaactcaagttggtggctgcatggccgaatagatttaaacaagcgctttttagaggtccgggtaaaacttgagtggaaaagattttcgtgcgccctgttactcattggtaaggaaaaatacattggggtgatgcatggaggtaaaatgttgatgaagggcgtagatttagtcaggaaaacaaactgtaaatttgtcaataccacagcctcccgattagtgaatctacttttcgaagacgacgaaatagcgatggctgcagatacagcaagagccggatttgaggattttgactgtcttccgagcggacttactaagcttggccggttgattgcagaggcaaggctagctattaccggcaacggactaaacattagggacttcataatgaccgcggaattaagtcgtgcggtggataactatgccagtttgaaattgcctcatctcacggtttatcaaaagaaagctgctccgcgcgaagaattacctccaattaaagaaagaatcgaatatgtcatcatagaaccgggacaatctgtcccgaatgatccagcaactgagtcttttcctaactaaaagaaacttcttaaatttcttagcctggcagaagatccagaatgggtcgtatcaaatggtctaaaattgaatgtagaatactactttgattcgcttattagaacattaagtgtaacattcaacgcaattttcggtgatgcaaaaacagcagaggatgttttaagaagctttattccagagaaaatacagttttctgagaaagttgcagaggctcttgcacgaaacaccgcgacatttgtatccatccagaagtaatcggagggctgtaagttgcaaatgttgaataaa

SEQ ID NO: 3 - DNA, 3021 bp, Gallid alphaherpesvirus 1
  misc_feature 1..3021: note = fully deoptimized version of SEQ ID NO: 2
  source 1..3021: mol_type = genomic DNA
atggatacgttttccagtgcgggtacgggtgctgctagggtcgcaccgataatcggcctcgggccgcattcgggtgcgtcgtattatacgtctgtgagagaatttccgtacgtatgtccaacatgcattaacggtgggggacgtataggaacgcagatcggtagggcgacttcgaaaccgacattctaccataacgaacgccaactcgatatacttacatcgacacatggcgcatggcccgtccgtatgaaatattggaacgctggtccgggtatgacgcatcacaacaagggagacgtaaagttttacgaatttcacgtatacgacctattcgaaaatactgaaatagcgcaatcgtgcgtacgttggatgcattcgcgatttctcgatacactaaaaccgaccggtactgtaattacgctagttggaaattcggcatgcggtaagcgcgtcgcggtacacgtgtacggatctctaccgtacttttttgtaaaaaaacgcgaaatcgatcaagcggctaaggtcacgaattcggaagagctagcgcatgcgctagcattagcgacgcgtaagacatcgttaaaaaattcgccgtttctagctgtatcagcggaatcgttttgcattgacgttgtacgtcgcagagatatatattattctgaatctgaagaggaggacttttatcgtgtaaaagtgtgtaacggtaaggtgatgaaattcatatgcgataactttttcccgagcgtaccgaaatacgaatcgaacgttgacgctattacgcgattcatcctcgataataacctaacgtctttcggttggtatcggtttagagctcaagggggcgcgctacaaattcgagatccaggacaacacgcgactagcgcggacgtcgaaataaattgtacggcttcgaatttggaactcggaaataatgtatcgtggccggattacaagttactatgtttcgatatcgaatgtaaggcgggtggggcgaacgagttcgcatttccgtctgctgaaaaggtagaagacctcgtaatccaaatatcggcggtaacatattcgttgttaacgaaagagaaagagcaagaaatcatcttttcgctaggtacgtgcgaactatcggaagattgttcggatggtataactgtatgcaagtgcggctctgaattcgaacttctactgtgctttatgacgttttttaagcaatatagtccggaattcgtatccggatataacatactagggttcgattgggcgtatctttcgaataagctatcgaaggtatatggtatgcgactcgacggatacgggaaagcgaactcgtggggtacgttcaaactccaagatccgagtgcgcgtggacttggtcggtttaaaaaggttaagatcaatggcgtagtcaatatcgatatgtttacgattagttacgaaaaacttaagttaccgtcttataaattaaatgcagtagcagaatgcgttctaggagaacaaaaaattgatctagcgtataaagacataccggtaatgttcgcagcgggcccgaaagaaaggggtaaaataggggaatattgtctacaagattctagactgtcgggtagtctattctttaagtttagtcctcatttagaaatgtccgctgtagctaaactagcgtgtataccgttaacacgagcaataggcgacggtcagcaactgcgcgtatatacgtgtctactccaacgatcgaccgcgtccggattcgtactaccggataaaaagtcggcgttttcgttcggttcgacccttgcgtccgacgcgtctgatgcacctgctacgagaaacgtagggtatcaaggtgccaaagtactagatccggaaatcggattccacgtaacgcccgtagtcgtattcgatttcgcctcgctatatccgtcgattatacaaagtaacaatctatgttatagtaccctaacgcacgacccggccgcactggcgggtctacaaccagaaaaagatttcttccaaattgatgtacaggggcgtaaattttacttcgtaagggaacatatacgcaaaagtttgctgtcggacctactaggcgattggttatcgatgaggaaagccgtgcgcgcgaaaattaagacagccgagacggaagaagagcgcatactattagataagcagcaagctgccctaagttgtctggaactctgttatggttttactggggtaatgcacggaatgcttccgtgtctcgaagtcgcttcgaccgtaaccgcgataggtcgcgacatgctattgcgtacaaaagcgcatatcgaaaaggagtggcgatcgggtaatcaatttgccgaaaaattcctaccgggttccgaacgtatacaattgaaccaatattctgtccgcgtaattaatggcgaaactgatagtgcgtattgcgaagtataccgaagtctacatttaaactttaattcaagttggtggttacacgggcgaatagacttaaacaagcggtttttagaggtccgggtaaaactcgaatggaaaagattttcgtgtgccctattactaattggtaaggaaaaatatataggcgtgatgcatggaggtaagatgctaatgaaaggcgtagatctcgtccgtaaaacgaattgtaagttcgtcaataccaccgcttcccgactcgtaaacctacttttcgaagacgacgagatagcgatggctgccgatacggcaagagcgggttttgaagatttcgactgtcttccgagcggtctaactaagcttggccgcctaatcgcagaagcacgtctagcgattacgggtaacggactaaacataagggattttataatgaccgcggaattatcgcgtgcggtcgacaattatgcgagtctaaagttgccgcatctaacggtttaccaaaaaaaagcggcgccacgcgaagagttaccgccaataaaggaaagaatcgaatacgtaatcatcgaaccgggacaatcggtcccgaacgatccagcaacggagtcgtttccgaat

SEQ ID NO: 4 - DNA, 5266 bp, Gallid alphaherpesvirus 1
  misc_feature 1..5266: note = wild type sequence of the glycoprotein J gene (US5)
  source 1..5266: mol_type = genomic DNA
atggggacaatgttagtgttgcgccttttcctacttgcagtagcggacgcggcgttgccgaccggcagattctgccgagtttggaaggtgcctccgggaggaaccatccaagagaacctggcggtgctcgcggaatcgccggtcacgggacacgcgacatatccgccgcctgaaggcgccgtcagctttcagatttttgcggacacccctactttgcgcattcgctacggcgctacggaggacgaacttgcactggagcgcgggacgtccgcctcagacgcggacaacgtgacattttcgctgtcatatcgcccgcgcccagaaattcacggagcatacttcaccataggggtattcgctactggccagagcacggaaagcagctattcggtcatcagtcgggtcttagttaacgcctctctggaacggtccgtgcgcctggaaacgccgtgcgatgaaaattttttgcagaacgagcctacatggggctcgaagcgttggttaggccccccgtcgccttatgtgcgagataacgatgtcgccgtgttgacaaaagcgcagtacattggggagtgctactccaactcggcggcccagacggggctcacgtctctcaacatgacctttttctattcgcctaaaagaatagtaaacgtcacgtggacaaccggcggcccctccccctcgcgcataacggtatactcgtcgcgggagaacgggcagcccgtgttgaggaacgtttctgacgggttcttggttaagtacactcccgacattgacggccgggccatgataaacgttattgccaattattcgccggcggactccggcagcgtcctcgcgtttacggcctttagggaaggaaaactcccatccgcgattcaactgcaccggatagatatgtccgggactgagccgccggggactgaaacgaccttcgactgtcaaaaaatgatagaaaccccgtaccgagcgctcgggagcaatgttcccagggacgactctatccgtccgggggccactctgcctccgttcgataccgcagcacctgatttcgatacaggtacttccccgacccccactaccgtgccagagccagccattactacactcataccgcgcagcactagcgatatgggattcttctccacggcacgtgctaccggatcagaaactctttcggtacccgtccaggaaacggatagaactctttcgacaactcctcttacccttccactgactcccggtgagtcagaaaatacactgtttcctacgaccgcgccggggatttctaccgagaccccgagcgcggcacatgaaactacacagacccagagtgcagaaacggtggtctttactcagagtccgagtaccgagtcggaaaccgcgcggtcccagagtcaggaaccgtggtattttactcagactccgagtactgaacaggcggctcttactcagacgcagatcgcagaaacggaggcgttgtttactcagactccgagtgctgaacagatgacttttactcagactccgggtgcagaaaccgaggcacctgcccagaccccgagcacgatacccgagatatttactcagtctcgtagcacgccccccgaaaccgctcgcgctccgagcgcggcgccggaggtttttacacagagttcgagtacggtaacggaggtgtttactcagaccccgagcacggtaccgaaaactactctgagttcgagtactgaaccggcgatttttactcggactcagagcgcgggaactgaggcctttactcagacttcgagtgccgagccggacactatgcgaactcagagtactgaaacacactttttcactcaggccccgagtacggtaccgaaagctactcagactccgagtacagagccggaggtgttgactcagagtccgagtaccgaacctgtgcctttcacccggactctgggcgcagagccggaaattactcagaccccgagcgcggcaccggaggtttatactcggagttcgagtacgatgccagaaactgcacagagcacacccctggcctcgcaaaaccctaccagttcgggaaccgggacgcataatactgaaccgaggacttatccagtgcaaacgacaccacatacccagaaactctacacagaaaataagactttatcgtttcctactgttgtttcagaattccatgagatgtcgacggcagagtcgcagacgcccctattggacgtcaaaattgtagaggtgaagttttcaaacgatggcgaagtaacggcgacttgcgtttccaccgtcaaatctccctatagggtagaaactaattggaaagtagacctcgtagatgtaatggatgaaatttctgggaacagtcccgccggggtttttaacagtaatgagaaatggcagaaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaatcgagcctagaataggtggtttcttcctacatgccacgcctcacgctcataatataaatcacatggaatagcataccaatgcctattcattgggacgttcgaaaagcatggcatcgctacttggaactctggctctccttgccgcgacgctcgcacccttcggcgcgatgggaatcgtgatcactggaaatcacgtctccgccaggattgacgacgatcacatcgtgatcgtcgcgcctcgccccgaagctacaattcaactgcagctatttttcatgcctggccagagaccccacaaaccctactcaggaaccgtccgcgtcgcgtttcggtctgatataacaaaccagtgctaccaggaacttagcgaggagcgctttgaaaattgcactcatcgatcgtcttctgtttttgtcggctgtaaagtgaccgagtacacgttctccgcctcgaacagactaaccggacctccacacccgtttaagctcactatacgaaatcctcgtccgaacgacagcgggatgttctacgtaattgttcggctagacgacaccaaagaacccattgacgtcttcgcgatccaactatcggtgtatcaattcgcgaacaccgccgcgactcgcggactctattccaaggcttcgtgtcgcaccttcggattacctaccgtccaacttgaggcctatctcaggaccgaggaaagttggcgcaactggcaagcgtacgttgccacggaggccacgacgaccagcgccgaggcgacaaccccgacgcccgtcactgcaaccagcgcctccgaacttgaagcggaacactttacctttccctggctagaaaatggcgtggatcattacgaaccgacacccgcaaacgaaaattcaaacgttactgtccgtctcgggacaatgagccctacgctaattggggtaaccgtggctgccgtcgtgagcgcaacgatcggcctcgtcattgtaatttccatcgtcaccagaaacatgtgcaccccgcaccgaaaattagacacggtctcgcaagacgacgaagaacgttcccaaactagaagggaatcgcgaaaatttggacccatggttgcgtgcgaaataaacaagggggctgaccaggatagtgaacttgtggaactggttgcgattgttaacccgtctgcgctaagctcgcccgactcaataaaaatgtga

SEQ ID NO: 5 - DNA, 2955 bp, Gallid alphaherpesvirus 1
  misc_feature 1..2955: note = fully deoptimized version of SEQ ID NO: 4
  source 1..2955: mol_type = genomic DNA
atggggacgatgctagtcttgcgcctattcctactagcagtagcggacgcagcgctaccgaccggtcggttttgccgagtttggaaggtgccgccagggggaacgatccaagagaatctggcggtactcgcggaatcgccggtaaccggtcatgcgacatatccgccgccggaaggcgcggtatcgttccaaatttttgcggatacccctacgttgcgcattcggtacggcgcgaccgaagacgaactagcgctcgaacgcgggacgagtgcgtccgacgcggataacgtaacgttttcgctatcatatcgcccgcgcccagaaatacacggtgcatactttacgataggggtattcgctaccggccaaagcaccgaatcgtcgtattcggtaatctcgcgggtattagtaaacgcgtcactggaacggtccgtgcgcctggaaacgccttgcgatgaaaactttctccaaaacgaaccgacttggggttcgaagcggtggttagggcccccgagtccttacgtgcgcgataacgacgtagccgtgttgacaaaagcgcagtatataggcgaatgctactcgaattcggcggcccaaacgggtctaacgtcgctaaatatgacgtttttctattcgcctaaacgtatcgtaaacgtcacgtggacaaccggaggtccgtccccgtcgcgcataacggtatattcgtcgcgggagaacggtcagccggtattgaggaacgtttctgacggattcctagttaagtatacgcccgatattgacggcagggcgatgataaacgttatcgcgaattattcgccagcggactcgggtagcgtcctcgcgtttacggcgtttcgcgaaggtaagttaccaagtgcgatacaattacaccgtatcgatatgtccgggactgagccgccggggactgaaacgaccttcgattgtcaaaaaatgatcgaaaccccgtatagggcgctagggagcaatgttccgcgagacgactctatacgtccgggtgccactctacctccattcgatacggcagcaccggatttcgatacaggtacttccccgactcccactaccgtaccggagccagccattactaccctaataccgcgatcgacttccgatatgggattcttctccaccgctcgtgctaccggttcagaaactctatcggtacccgtacaggaaacggatcgtactctttcgacaactcctttaacccttccgctgacgcctggtgaatcagaaaatacgctatttcctacgaccgcgccgggtatatctaccgagaccccgagcgcggcacatgaaactacacaaacccaaagtgcagaaacggtcgtctttactcaaagtccgtcgaccgagtcggaaaccgcgcgatcccagtcgcaagaaccgtggtattttactcagacgccgagtactgaacaagcggcactaactcaaacgcaaatagcagaaacggaagcgttgtttactcagactccgagtgctgaacaaatgacttttactcagactcccggtgcggaaaccgaagcgcctgcccagacccctagcacgatacccgaaatatttactcagtctcgttcgacgccccccgaaacggctcgcgcgccgagcgcggcgccggaagtctttacacagagttcgagtacggtaacggaagtgtttactcagaccccgagcaccgtaccgaaaactactctaagttcgagtactgaaccggcgatatttactcggactcagtcggccggaactgaagcctttactcagacttcgagtgccgaaccggatactatgcgaactcagagtactgaaacgcattttttcacgcaggcgccatcgacggtaccgaaagcgactcagactccgagtacagagccggaggtgttgactcagagtccgagtaccgaaccggtgccgtttacccggacgctaggcgcagagccggaaattactcagaccccgagcgcggcaccggaggtttatactcgaagttcgtcgacgatgccagaaactgcgcagagcactccgttagcgtcgcaaaatccgacttcgtcgggtacgggtacgcataatactgaaccgcgtacttatccggtgcaaacgacaccacatacccaaaaactatatacagaaaataagacgttatcgtttcctactgttgtatcggaatttcacgaaatgtcgaccgcagaatcgcagacgccgctattggacgtaaagatagtcgaggttaagttttcgaacgacggtgaggtaacggcgacatgcgtttccaccgttaaatcgccctatagggtcgaaactaattggaaagtcgacctagtcgatgtaatggacgaaatatctggtaatagtccagcgggcgttttcaatagtaacgagaagtggcagaaacagctgtattacagagtaaccgatggtagaacgtcggtacaattgatgtgtctatcgtgtacgtcgcattctccggaaccgtactgtcttttcgacacgtctctaatagcgagggaaaaagacatagcgcccgaactatactttacgtctgatccgcaaaccgcatattgtacgataactctgccgtccggagtcgttccgcgattcgaatggagcctaaataatgtatcgctgccggaatacctaacggccacgaccgttgtttcgcataccgctggtcaaagtaccgtatggaaaagcagtgcgcgtgcgggcgaagcgtggatttctggtagggggggcaatatatacgaatgtacggtcctcatatcagacggcacgcgcgtaactacgcgtaaggagcggtgtctaacaaatacgtggatagcggtggaaaacggcgctgctcaagcgcaactgtattcactattttcgggactcgtgtcgggattatgcggtagcatttcggcgctatacgcgaccctatggaccgccatatacttt

SEQ ID NO: 6 - DNA, 3000 bp, Gallid alphaherpesvirus 1
  misc_feature 1..3000: note = wild type sequence of sORF1
  source 1..3000: mol_type = genomic DNA
atgaccttgccccatcgattaacgaaaagacctttcgcgcgtcgattctgctcggtctttgtgatacattatagtgagactaaactcgaccgatataacaagacaatgttactctatagaccggactcaaccatgcggcatagcggaggcgacgcaaatcacagagggataaggccgaggcggaaatctattggagcgtttagcgcgcgcgaaaagactggaaaacgaaatgcgctgacggaaagcagctcctcctccgacatgctagatccgttttccacggataaggaatttggcggtaagtggacggtagacggacctgccgacattactgccgaggtcctttctcaggcatgggacgttctccaattagtgaagcatgaagatgcggaggaggagagagtgacttatgagtccaaaccgaccccgatacagccgttcaatgcctggccggacgggccgagttggaacgcgcaggattttactcgagcgccaatagtttatccctctgcggaggtattggacgcagaggcgttgaaagtaggggcattcgttagccgagttttacaatgtgtaccgttcacgcgatcaaagaaaagcgttacggtgcgggatgcgcagtcgtttttgggggactcgttctggagaataatgcagaacgtttacacggttgtcttacgacagcacataactcgactcaggcacccttccagcaaaagcattgttaactgcaacgaccctctatggtacgcctacgcgaatcaatttcactggagaggaatgcgcgtgccgtcgcttaaattagcctctcccccggaggagaatattcaacacggcccaatggccgccgtttttagaaacgcgggggctggtctgttcctgtggcctgccatgcgcgcagcctttgaagagcgcgacaagcgactgttaagagcatgcctgtcttcactcgatatcatggacgcagccgtcctcgcgtcgtttccattttactggcgcggcgtccaagacacctcgcgcttcgagcctgcgctgggctgtttgtcagagtactttgcactagtggtgttactggccgagacggtcttagcgaccatgttcgaccacgcactggtattcatgagggcgctggcagacggcaatttcgatgactatgacgaaactagatatatagaccccgttaaaaacgagtacctgaacggagccgagggtactctgttacggggcatagtggcctccaacaccgctctggcggtggtttgcgcaaacacctattcgacgataagaaaactcccgtccgtggcaactagcgcgtgcaatgttgcctacaggaccgaaacgctgaaagcgaggcgccctggcatgagcgacatataccggatattacaaaaagagtttttcttttacattgcgtggctccagagggttgcaacacacgcaaatttctgtttaaacattctgaagagaagcgtggatacgggggccccgccatttttgttcagggccagctcggagaagcggctgcagcagttaaataaaatgctctgcccccttctcgtgccgattcaatatgaagacttttcgaaggccatggggtctgagctcaagagggaaaagttagagacattcgttaaagctatttccagcgacagggacccgagggggtccttaagatttctcatttcggaccatgcaagggaaattattgcagacggagtacggtttaagccggtgatagacgagccggttcgggcttcagttgcgctgagtaccgctgccgctgggaaagtgaaagcgcgacgcttaacctcagttcgcgcgcccgtaccgggcgcaggcgccgtttccgcgcgccggaaatcggaaatatgataaaaatgcttggcatttgcgggcgaagaggcgtgatctgaagggctccacaatgacgtaactgagctacgcatccctataaagtgtacccgctgaccgctagcccatacagtgttacaggaggggagagagacaacttcagctcgaagtctgaagagacatcatgagcggcttcagtaacataggatcgattgccaccgtttccctagtatgctcgcttttgtgcgcatctgtattaggggcgccggtactggacgggctcgagtcgagccctttcccgttcgggggcaaaattatagcccaggcgtgcaaccgcaccacgattgaggtgacggtcccgtggagcgactactctggtcgcaccgaaggagtgtcagtcgaggtgaaatggttctacgggaatagtaatcccgaaagcttcgtgttcggggtggatagcgaaacgggcagtggacacgaggacctgtctacgtgctgggctctaatccataatctgaacgcgtctgtgtgcagggcgtctgacgccgggatacctgatttcgacaagcagtgcgaaaaagtgcagagaagactgcgctccggggtggaacttggtagttacgtgtctggcaatggatccctggtgctgtacccagggatgtacgatgccggcatctacgcctaccagctctcagtgggtgggaagggatataccgggtctgtttatctagacgtcggaccaaaccccggatgccacgaccagtatgggtacacctattacagcctggccgacgaggcgtcagacttatcatcttatgacgtagcctcgcccgaactcgacggtcctatggaggaagattattccaattgtctagacatgcccccgctacgcccatggacaaccgtttgttcgcatgacgtcgaggagcaggaaaacgccacggacgagctttacctatgggacgaggaatgcgccggtccgctggacgagtacgtcgacgaaaggtcagagacgatgcccaggatggttgtcttttcaccgccctctacgctccagcagtagccacccgagagtgttttttgtgagcgcccacgcaacatacctaactgcttcatttctgatcaattattgcgtattgaataaataaa

SEQ ID NO: 7 - DNA, 1869 bp, Gallid alphaherpesvirus 1
  misc_feature 1..1869: note = fully deoptimized version of SEQ ID NO: 6
  source 1..1869: mol_type = genomic DNA
atgacgctaccgcatcgcctaacgaaacgacctttcgcgcgtcgattctgttcggtattcgtaatacactattcggaaactaaactcgatcggtataataagacgatgctactctatcgaccggactcaacgatgcgtcattcgggtggcgacgcaaatcataggggtatacgaccgcgacggaagtcgattggtgcgttttcggcacgcgaaaaaactggtaagcgtaatgcgctaaccgaaagcagctcgtcgtccgatatgctagatccgttttcgaccgataaggaattcgggggtaagtggacggtcgacggtccggccgatataaccgcggaggtcctatcgcaagcgtgggacgttctccaactcgtaaaacatgaagacgcggaagaggaacgcgtgacgtacgaatcgaaaccgactccgatacaaccgtttaatgcctggccggacgggccgagttggaacgcgcaggactttacgcgagcgccaatcgtatatccgtctgcggaagtactcgacgcagaagcgcttaaggtcggggcgttcgtaagccgcgttttacaatgtgtaccgttcacgcgatcgaagaaatcggttacggtgcgcgatgcgcaatcgtttctaggggattcgttttggcgtataatgcaaaacgtatatacggtcgtcttacgccaacacattactcgattgcggcatccgagttcgaaatcgatcgtaaattgtaacgacccgttatggtatgcgtacgcgaatcaatttcattggagaggtatgcgcgtgccgtcgctaaaactagcgagtccgccggaggagaatatacaacacggcccgatggccgccgtatttcgtaacgcgggtgcgggtctattcctatggccggccatgcgtgcagcattcgaagagcgcgataaacgactgttacgcgcatgcctatcgtcactagacataatggacgcagccgtcctcgcgtcgtttccattttattggcgaggcgtacaagatacgtcgcgcttcgaaccggcgctagggtgtctatcggaatacttcgcactagtcgtgctcctagccgagacggtattagcgactatgttcgaccatgcgctggtctttatgagggcgctggcggacggtaatttcgatgattacgacgaaacgcgatatatagacccggttaagaacgaatacctaaacggtgccgaaggtacgctattacgcggtatagtagcgtcgaacaccgcgctagcggtggtttgcgcgaatacgtattcgacgatacgtaagttaccgtccgtagcaactagcgcgtgtaatgtagcgtatcgtacggaaacgctgaaggcgaggcgaccgggcatgtccgacatataccgtatattacaaaaagagttttttttctatatagcgtggttacaacgcgttgcgacacatgcaaatttttgcctaaacattctgaaaagatcggtcgatacgggggcgccaccgtttttgttccgcgccagctcggaaaaacgcctacaacaattgaataagatgctatgcccgttactcgtaccgattcaatacgaagacttttcgaaggcgatgggttctgaactcaaaagggaaaaattagagacattcgttaaagctatatcgagcgatcgcgatccgcgaggttccttacggtttctaatctcggatcatgcgcgcgaaattatagcagacggcgtacggtttaaaccggtaatcgacgagccggttcgggcatcggttgcgctaagtaccgcggctgcggggaaggtaaaagcgcgacgattgacgtcggttcgcgcgccggtaccgggtgcaggtgcggtatccgcgcgccgtaaatcggaaatc

SEQ ID NO: 8 - DNA, 1260 bp, Gallid alphaherpesvirus 1
  misc_feature 1..1260: note = wild type sequence of the glycoprotein C gene (UL44)
  source 1..1260: mol_type = genomic DNA
atgcagcatcagagtactgcgctagtttcgagtatacttttgctcttgagcctgcaaagccttgcgtttgaatttttctgtgatccgccacacgtttttcgagggcagctcggtgaccccattctattgcaatgcttcagcgacagacctctaacccacgaagaatctgtaaaagtagaagtaattcgacacccagccagcttagttgaaactgcgctaagcgcctacgggatccccccttcgctagatccatggagagctactccaagaactctctacacatatgatgccgctactgattcaatcaaggacctaggatacattggtgaagatggaattaacccaccatatttggacgactgtcgttcaggttttttcaatgtctctatcaagtctagcatgagatctcacatggcgcgttatcagtggaccgcaagtcgagggtctacaaaactaaatagctcttttatcgacgtctttttggcaagaccacctacaactgtccgcatcaaatcagaagaactgtacgaagactcagataaggcttcgcacttaagtgttgaagcgcttggcgcttatcctccatctgctgcgctgggtacatggatgatacataatgcatctcttgctgaaaaatacagtttagaaagaagagttctttatgcatcaggagagaatggatcggtggatcagacatgggaactggaaatacgtggagaagccagccagcccctcccttccaaaattcaatttgtatatcgatggacccctcctgaggactttgaaatgctacgacctgaaactcgcttgttaaggttgactcccagctggattagcaagccccgcatcacggtacaattcgtccctcctgcctatgccctgtgtagagcagctaatattatagacggccgaggatttattgaatggatcgtagataatagaatttcgacgagcccacaccagacctttgttttggatgagcccgaggggaaaaatatcgttacactaatggacgtcataaaactaccaccggaggatacatttcaatctgcctctaattacgtgtgcgtcataagaggctatgaacatgcatacagatatctcaacgcctccttaatgatagataatctgccaatgcggcaaggattccccgcagtcgctgcgatttttattataattagtatcgcttttgtgggtgggttactagttgcttgcttgggcgcatggtgctggaagacaacataaacgctcatttaataa

SEQ ID NO: 9 - DNA, 1242 bp, Gallid alphaherpesvirus 1
  misc_feature 1..1242: note = fully deoptimized version of SEQ ID NO: 8
  source 1..1242: mol_type = genomic DNA
atgcaacaccaatcgactgcgctagtttcgtcgatccttctactactatcgctacaaagcctagcgttcgaattcttttgtgatccgccacacgtatttcgcggtcaactaggcgacccgatactactccaatgtttttcggatcgaccgttaacgcacgaagaatcggttaaggtcgaggtaatacgccatccggcttcgttagttgaaaccgcgctatcggcatacgggatcccccctagtctcgatccgtggcgtgcgactccgcgtacgctatatacgtacgatgcagcgaccgattcaattaaggacctagggtatataggcgaagacggaataaatccgccgtatctagacgattgtcgatcgggtttttttaacgtatcgatcaagtcgagcatgcgatcgcatatggcgcgatatcaatggaccgcgtcacgcgggtctacgaaattgaattcgtcttttatcgacgtattcctagcgcgaccacctacgaccgtacgcataaaatcggaagagctgtacgaagattcggataaggcgtcgcatctaagtgtcgaagcgcttggcgcgtatcctccatctgcagcgctgggtacgtggatgatacataatgcgtcgcttgcggaaaagtattcgctcgaacgtcgcgtactatacgcgtcaggcgaaaacggttcggtggaccaaacgtgggaactcgagatacgcggcgaagcctcgcaaccgttaccgtcgaaaatccaattcgtatatcgttggacgcctcctgaagacttcgaaatgctacgaccggaaacgcgactattacgcctaacgcccagttggatatcgaagccgcgtataacggtacaattcgtaccgccagcctatgcgctatgtagagcagcgaatataatcgacggtagggggtttattgaatggatagtcgacaatcggatttcgacgagtccgcatcaaaccttcgtactcgatgaaccggaaggtaagaatatcgtaacgcttatggacgtaatcaagttaccgccggaggatacgttccaatcggcttctaattacgtgtgcgtaataagaggctacgaacatgcatatcggtatctaaacgcctcgctaatgatcgataacctaccgatgcgtcaagggtttccggcggtcgctgcgatattcataatcatatcgattgcgtttgtaggcggactactcgtagcgtgtctaggcgcatggtgttggaaaacgact

SEQ ID NO: 10 - DNA, 3161 bp, Gallid alphaherpesvirus 1
  misc_feature 1..3161: note = wild type sequence of the UL37 gene
  source 1..3161: mol_type = genomic DNA
atgcaggccatcacggatagcctcaaggcttgccttgaagctgtacacaccgactgcagtaaagttccgggtgttacttcagccctcaccggaattctaatctccagaaacagaattccacttgaagatttggagaaagtagaagctaggaaaaacatggttgaaactataatgctcgcatgcacaatggcgcctccggcagtagcggacgttacattgaaattatttgcacgctctcttattagccgcatatctgttccatggaactctggagaagatttcagaataattcaatatttgagagaagcatttactggactgcaacatgacctagaaacagctgtacgtaaagaatttcctgggttaaatgatccaaatgtagaatattggagtctaattagtggctggttaacggaatttgggagtttggcaagattggtagttgacgagcgacaattgttcgagacatccggagaccgactcgagccaaccaaactcctatctcctttaattgaaaattatccacttttatacgaccaccaaatggttcaagacgggatacagtatcttggaacaaagctacaaggcttagtgggataccatgcaatgttaaattatatcacggcttcaaccgggctacccaaaactaaagcgctgatgaccctctctatggtcagagagtattttgaacctacagtaaaacccccttatcggaagtttccacagacggcgctagctgcctttgatctggacacagattcggccaagtcatggatcgctaaagatttagactcaagatacttttctatctcgcaactgtacgccaatgctgcatacagtcgagacccactactcgtgttaggagatcctccgaagacgcatgttcctggacatgtagtttgctggcggcacgacttagatagtctattaactattcccattaaacgcctcgccctagatgtcccaaccatcgtacagcttttcgaggacgcgcctaataacttgacagaggaacaaaaggagaccctaactgcttatacaacaagtaccgccgatatgatggggcgttcgccggacttggccgctaccggtggcccagaggaagtaattcgtactttagtgcttagggggtttaccaagagcaactgcgaactgtatatccaaaaggctgaggcgctttggggtgaagatactgacggaacaatcattggagatttcctaggagccgttgtatatacatcacttataggactcgctgcgcactcaatgtatacatataatccgcggacgctacagtattcagggcattacgctgggcttatatctgattggggagacccatattcgcatcttctccggcgggtaggggtgtctgaaagcgaactactactaccactacgatcatttgccccagccccatcaaaaaatgctctaaacatcattagggacactctggcctcatttatagatgggagcgatggaggacccgaggcgcttggaaatttgggggtgttcaattttgttcaacacctcatatctgacttaggaggaaagacccaggaaaatttggattggctcgaacgcagggaaaaggccaaagataaggggtatgcaagtgccgacaaacaatcctacacttatggatttgaccaaaccgtaaaattagtccaggccattgaagttgggggcgcggagcctgaagagatatgttcgcgtaatggaacggacccgaaaatcgatatgcaactcttttccaatctttccgtagcgaccgtattgagagacattgtttacgcagtatcaacttatccattcaccgttgacgcggtacaacacgcggtaaagcttttatcggggatagagattagggctctcagaaaaccgtctgatactcagaagtacaggcgagcaattttggaatttcagttagcggtctccccatttattacaaagaccaatccgacagaaaagatatccatcgaaagtgcaagcgcgatagaaaaggcattgcttaaaatttctgacgcatgcgacaggagcctcgacactcttccagaaacatttaagaaaacagtccagcctgtccctgccgtcgataccatgagatcaaagttcatttcccaggcattcgagcaagctgcaacgagagaccttgaggaggtttccaagactctaaataacgcatctatgcagatatctactgcagtgcaaatactactttctaagactgctaacataagaccactgctttcacagtcagcaaatgcggattttttgggcaatgtttctttctcaagttgccgtgacaaaaaagaactaggacactggaaggacgatactgtaatgcaggccatatggaaagccgtcaattttgcagaaactgcaatccgacacacagaaaaagctattcgggcattagattccacaaaggttaaaacgcatgcacttgagaccacatactggcagcttgcgggttatgctcaaacacgctctcacatttgtaggaaagtagctgaagaattatctggcatagcggacgcgaggaatactgtcgtccagcgttcgtggacattatgtcgttcactcgcaggcgtcggggtaaaaagggcccgcgatgtggtaaaagcctggaaaactttttcagataatgaagctgctctcgagacatcttaatccaaaaccctgaagatgtagttaaagttattggcgcggcatcagctctgctagatatagaggagggagggaccacgacccttgaaggaaaattggaagaaattgaaatttctgccccttctaaagaagatacagcgtcagctcttactcttttagggccgcctgatgccaggggatatcaagatacatcatcaaatcaatttattaccgaggcgtcagacattacttcgtgggataaaaccaacaaagccccgctctatgttcgacactcgacaaaagaagggatagacaatgggtgtgtagacacacttccagacaaactaatctcatcagagactcttctgacagtttccgatcgagttttggaaagtgtaaatgtaaaaatgtttgaataaagaaagactaatgagtaattgagtggtgtgtatttatcgcgttgttgtgtgcggttggctagacatttacgaaactcttcccaaacaaataaa

SEQ ID NO: 11 - DNA, 2670 bp, Gallid alphaherpesvirus 1
  misc_feature 1..2670: note = fully deoptimized version of SEQ ID NO: 10
  source 1..2670: mol_type = genomic DNA
atgcaagcgatcacggattcgctaaaggcgtgccttgaagctgtacacaccgattgttcgaaggttccgggtgtaacgagtgccctcaccggtatactcatatccagaaaccggattccgttagaagatttggaaaaggtagaagctcgtaagaatatggttgaaactattatgctagcgtgtacgatggcgcctccggcagtagcggacgttacgttgaaattatttgcgcgatctcttatcagccgtatatcggtaccgtggaattctggcgaagactttcgcataatccaatatttgagagaagcatttacgggtctgcaacatgacctagaaacagcggtacgcaaagagtttcctggtctaaacgatccaaacgtcgaatattggagtctaatttcgggttggttaacggaattcggtagtctagcgcgactcgtagttgacgaacgccaactattcgaaacatccggagatcgactcgagccgaccaaactcctatcgccgttaattgaaaactatccacttctatacgatcaccaaatggttcaagacgggatacaatatctcggtacgaagctccaagggttagtggggtatcatgcgatgctaaattatatcaccgcttcgaccggtctacccaaaacgaaagcgcttatgacgctatcgatggtcagagaatatttcgaacctacagttaagccgccttatcgtaagtttccacaaaccgcgctagcggcatttgatctggacacggatagtgccaagtcatggatcgctaaagatttagattcacgatatttttctatctcgcaactgtacgccaatgctgcatacagtcgcgatccactactcgtactaggagatccgccgaagacgcacgtacctggtcatgtcgtatgttggcgacacgacctcgatagtctattaacgataccgattaaacgcctagcgctagatgtcccgaccatcgtacaacttttcgaggacgcaccgaataacttgaccgaagagcaaaaggaaacgctaactgcttatacgactagtaccgcggatatgatgggtcggagtccggacttggcggctacgggcggcccagaggaggtaattcgtacgctagtccttcgcggttttacgaagtcgaattgcgaactatatatacaaaaagctgaagcgctatggggtgaagatactgacggtacaatcataggcgatttcctcggagccgtcgtatatacgtcactaataggactagctgcacattcgatgtatacgtataatccgcgtacgctacagtattcgggtcattatgcgggtcttatatctgattggggcgacccgtattcgcatctattgcgtcgggtaggcgtatcggaatcggaactactactaccattacgatcgttcgcgccagccccatcaaaaaacgctctaaacataataagggacacgctggcgtcgtttatcgacggttcggacgggggtcccgaggcgctaggtaacttgggggtattcaatttcgttcaacacctaatctcggacctaggcggaaagacccaagaaaatttggattggctcgaacgtagggaaaaagccaaagataaggggtatgcatcagcggataagcaatcgtatacttacggattcgatcaaacggtaaagttagtccaggccattgaagtcgggggtgcggaacctgaagagatatgttcgcgtaacggtacggatccgaaaatcgatatgcaactcttttccaacctatccgtagcgaccgtattaagagacatcgtttacgcggtatcaacgtatccgttcacggtcgacgcggtacaacacgcggttaagctattatcggggatcgaaattcgcgcgctcagaaaaccgtccgatacgcagaagtaccgacgtgcgatactcgaattccagttagcggtaagtccgtttattacaaagaccaatccgaccgaaaagatatcgatcgaatcggcgagcgcgatagaaaaggcactacttaagatttctgacgcatgcgataggagcctagatactctaccggaaacgtttaagaaaacggtccagcctgtacctgccgtcgatacgatgagatcaaaattcatttcccaagcgttcgaacaagctgctacgagagatcttgaagaggtttccaagacgctaaacaatgcgtcgatgcaaatatcgaccgcagtacaaatcctactttcgaagaccgctaatatacgaccgctgctatcgcaatcagcgaatgcggattttttgggtaatgtttcgttttcgtcgtgtcgtgataaaaaagaattagggcattggaaggacgatacggtaatgcaggcgatatggaaagccgtaaactttgcggaaaccgcaatacgccatacagaaaaagcgattcgggcactcgattcgacaaaagttaaaacgcatgcgcttgagacgacgtattggcaacttgcgggttatgcgcaaacgcgatcgcatatatgtcgtaaggtcgcggaagagctatctggtatagcggacgcgaggaatacggtcgtacagcgatcgtggacactatgtcgatcgctcgcgggcgtcggcgtaaaaagggcacgcgacgtagtcaaagcctggaaaactttttcggataatgaagcggctctcgagacgagt

SEQ ID NO: 12 - DNA, 1662 bp, Avian orthoavulavirus 1
  misc_feature 1..1662: note = wild type sequence of the fusion gene
  source 1..1662: mol_type = genomic DNA
atgggctccagaccttctaccaagaacccagcacctatgacgctgactatccgggttgcgctggtactgagttgcatctgtccggcaaactccattgatggcaggcctcttgcagctgcaggaatcgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggtgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcggttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtga

SEQ ID NO: 13 - DNA, 1662 bp, Avian orthoavulavirus 1
  misc_feature 1..1662: note = fully optimized version of SEQ ID NO: 12
  source 1..1662: mol_type = genomic DNA
atgggcagccgcccttctaccaagaacccagcaccaatgacgctgaccatccgggttgccctggtactgagctgcatctgcccagccaacagcattgatggccggcctctggcagcagcaggaattgtggtgacaggagataaagccgtgaacatctacacctcaagccagacaggcagcatcatagtgaagctgctcccgaacctgcccaaggacaaagaagcctgtgccaaagctcccctggatgcctacaacagaacattgaccactctgctgacaccactgggagacagcatccgcaggatccaggagtctgtgaccaccagtggaggaggaaggcagggccgcctgattggagccatcattggcggtgtggctcttggagttgcaactgccgcacagataacagcagccgcagcactgatacaggccaaacagaatgctgccaacatcctgcggctgaaagagagcattgccgccaccaatgaggctgtgcatgaagtgacagatgggctgagccagctagcagtggctgtggggaagatgcagcagtttgtgaatgaccagtttaacaaaacagcacaggaactggactgcatcaagattgcacagcaagtgggtgtggagctgaacctgtacctgaccgagctgacaacagtatttggaccacaaatcacctctccagctttaaacaagctgactatccaggcactgtacaacctggcaggtggaaacatggactacctgttgaccaagctgggagtaggcaacaaccagctgagcagcttaattggcagcggcttaatcaccggaaaccccatcctgtatgactcacagacacagctcctgggcatccaggtgactctgccttctgtcgggaacctaaataacatgcgtgccacctacctggaaacactgtctgtcagcacaaccagaggatttgcctcggcactggtgcccaaagtggtgacacaggtcggtagcgtgattgaagagcttgacacctcctactgtattgaaacagacctggacttatactgcacaagaattgtcaccttccccatgagccccggcatttacagctgcctgagcggcaacacatcggcctgcatgtacagcaaaaccgaaggagcacttaccaccccctacatgactatcaaaggatcagtgattgccaactgcaagatgaccacatgccgctgcgtgaatcctccaggtatcatcagccagaactatggagaagctgtgtccctaattgataagcagtcatgcaatgtgttaagcctgggcggcatcaccttacggctgagtggagaatttgatgtcacttatcagaagaacatcagcatacaggacagccaggtcatcataacaggcaatctggacatctcaacagagcttggaaatgtcaacaactcgatcagcaatgctttgaataagctggaggaaagcaacaggaagctagacaaggtgaatgtgaaactgaccagcacttctgctctgatcacctacattgtgttgactatcatatctcttgtgtttggcatcctgagcctcatcctggcctgctacctgatgtacaagcaaaaagcgcaacaaaagacactgttatggctggggaacaacacactggaccagatgagagccaccaccaagatgtga

SEQ ID NO: 14 - DNA, 2263 bp, Homo sapiens
  misc_feature 1..2263: note = Human FAP
  source 1..2263: mol_type = genomic DNA
atggattggacctggatcctgttcctggtggctgccgctacaagggtgcactccctcaggccctccagggtccacaactccgaagagaatactatgagggccctcacactgaaggatatcctgaacggcacattctcctataaaaccttctttccaaattggatcagcggccaagagtacctccaccagagcgccgacaataacatcgtgctgtacaatatcgagaccggacaaagctatacaatcctctccaacagaaccatgaaatccgtcaatgcctccaattatggactctccccagacagacaattcgtctacctggagtccgactactccaagctctggagatatagctataccgccacatattacatttacgacctcagcaacggcgaattcgtgagggggaacgagctccccaggcccatccagtacctgtgttggagccccgtgggcagcaagctcgcctacgtctaccagaacaatatctatctcaagcaaaggcccggcgaccctcccttccagattacctttaatggcagagagaacaagatctttaacgggatccctgattgggtctatgaagaggaaatgctggccactaagtacgccctgtggtggagccccaacggcaagttcctggcctacgctgagtttaacgatacagacattccagtgatcgcttatagctactatggcgatgagcagtatcccagaaccatcaacatcccctaccccaaggccggcgccaagaatccagtcgtgagaattttcatcattgacacaacctaccccgcctacgtcggcccccaggaagtccctgtgcctgctatgatcgccagctccgactactatttcagctggctcacttgggtcaccgatgagagagtgtgcctccagtggctcaagagggtccagaacgtctccgtgctcagcatttgcgatttcagagaagactggcagacttgggattgtcccaaaacccaggaacacatcgaggaatccaggaccggctgggctggagggtttttcgtcagcaccccagtcttcagctacgatgctatcagctactataaaatcttttccgataaagacggctacaaacatatccactatatcaaggatacagtcgagaacgccatccagatcacatccggcaaatgggaggccattaatattttcagagtgacacaggacagcctgttttacagctccaacgagttcgaagagtatcctggcaggagaaacatttatagaatcagcatcggaagctatccaccctccaagaaatgcgtcacatgccacctgagaaaagagagatgccagtattacactgctagcttcagcgattatgccaagtattacgctctggtgtgttatgggcctggaattcccattagcactctgcacgatggcagaactgaccaggaaattaaaatcctggaggaaaacaaggaactggaaaacgccctgaagaacatccagctgcccaaagaagagatcaagaaactcgaggtggacgagattactctgtggtacaagatgattctcccaccccaatttgacagaagcaagaaatatcccctcctgatccaggtctacggcgggccctgttcccagagcgtgaggagcgtgttcgccgtgaactggatttcctacctggcctccaaggaaggaatggtgatcgccctggtggacggaagaggaaccgccttccaaggggataaactcctgtacgccgtgtatagaaaactgggggtgtacgaagtcgaggatcagatcactgccgtgaggaagtttatcgaaatgggcttcattgacgagaagaggatcgccatttggggctgggcttacggaggctacgtgtccagcctcgctctggctagcggcaccgggctctttaagtgcgggatcgccgtggcccccgtctccagctgggagtattacgctagcgtgtacacagaaagattcatgggcctgccaacaaaagatgacaatctggaacactataagaatagcactgtcatggctagggctgagtactttaggaacgtcgattatctcctgattcacggcactgctgacgataacgtgcacttccagaactccgcccagattgctaaagctctcgtgaacgctcaggtggacttccaggctatgtggtactccgaccaaaatcatgggctcagcggactcagcacaaaccacctgtatacccacatgactcattttctgaaacaatgtttttccctgagcgactaaa

SEQ ID NO: 15 - DNA, 2259 bp, Homo sapiens
  misc_feature 1..2259: note = Codon pair optimized human FAP
  source 1..2259: mol_type = genomic DNA
atggactggacctggatcctcttcctggtggctgctgccacccgggtgcacagcctgcggcccagccgggtgcacaacagcgaggagaacaccatgagagccctgaccctgaaggacatcctgaatggcaccttctcctacaagaccttcttccccaactggatcagcggccaggagtacctgcaccagagcgccgacaacaacatcgtgctgtacaacattgaaacaggccagagctacaccatcctgagcaacaggaccatgaagtctgtgaatgccagcaactatggcctgtccccagacaggcagtttgtgtacctggagagtgactacagcaagctgtggagatacagctacacagccacctactacatctatgacctgagcaatggagagtttgtgagaggaaatgagctgccccggcccatccagtacctgtgctggagccctgtgggcagcaagctggcctacgtgtaccagaacaacatctacctgaagcagaggcctggggacccccccttccagatcaccttcaatggccgggagaacaagatcttcaatggcatcccagactgggtgtatgaggaggagatgctggccaccaagtatgccctgtggtggagccccaatggcaagttcctggcctatgccgagttcaatgacacagacatccctgtgattgcctacagctactatggagatgagcagtaccccaggaccatcaacatcccctaccccaaggccggggccaagaaccctgtggtgcgcatcttcatcattgacaccacctacccagcctacgtgggcccccaggaggtgcctgtgcctgccatgattgccagcagtgactactacttctcctggctgacctgggtgacagatgagcgggtgtgcctgcagtggctgaagagggtgcagaatgtgtctgtgctgagcatctgtgacttccgggaggactggcagacctgggactgccccaagacccaggagcacatcgaggagagcagaacaggctgggccgggggcttctttgtgtccacccctgtgttctcctatgatgccatcagctactacaagatcttcagtgacaaggatggctacaagcacatccactacatcaaggacaccgtggagaatgccatccagatcaccagcggcaagtgggaggccatcaacatcttccgggtgacccaggacagcctcttctacagcagcaatgaatttgaggagtacccaggaagaagaaacatctaccgcatcagcattggcagctacccccccagcaagaagtgtgtgacctgccacctgaggaaggagcgctgccagtactacacagcctccttcagtgactatgccaagtactatgccctggtgtgctatggccccggcatccccatcagcaccctgcatgatggaagaacagaccaggagatcaagatcctggaggagaacaaggagctggagaatgccctgaagaacatccagctgcccaaggaggagatcaagaagctggaggtggatgagatcaccctgtggtacaagatgatcctgcccccccagtttgaccgcagcaagaagtaccccctgctcatccaggtgtatggaggcccctgcagccagtctgtgcgcagcgtgtttgctgtgaactggatcagctacctggccagcaaggagggcatggtgattgccctggtggatggccggggcacagccttccagggggacaagctgctgtatgctgtgtacaggaagctgggcgtgtatgaggtggaggaccagatcacagctgtgaggaagttcatcgagatgggcttcattgatgagaagagaattgccatctggggctgggcctatggaggctacgtgtccagcctggccctggccagcggcaccggcctcttcaagtgtggcattgctgtggcccctgtgtcctcctgggagtactatgcctctgtgtacacagagcgcttcatgggcctgcccaccaaggatgacaacctggagcactacaagaacagcaccgtgatggcccgggccgagtacttcagaaatgtggactacctgctcatccacggcacagcagatgacaacgtgcacttccagaacagcgcccagattgccaaggccctggtgaatgcccaggtggacttccaggccatgtggtacagtgaccagaaccacggcctgagcggcctgagcaccaaccacctgtacacccacatgacccacttcctgaagcagtgcttcagcctgagtgac

SEQ ID NO: 16 - DNA, 2260 bp, unidentified
  misc_feature 1..2260: note = Chicken FAP
  source 1..2260: mol_type = unassigned DNA
atggattggacatggattctgttcctggtggccgctgccaccagagtgcacagcctcctgccctccaaagtcgtgacaactgtggacggcccaagggctctcaccctcgatgactatctcaacggaaactttcagtacaagacatatttcccctattgggtctccgattccgaatacctccaccagaaccaggaagatgacattatcctcttcaatgtggacatgaattacctcactaccatcatgaccaactccaccatgaagcaggtgaacgccagcaattacgtgatgagctccgacaagtattttatcgctctggaaagcaattattccaagctctggagatatagctacaccgccagctatcacatttatgatctcatctacggaggcttcgtgaccgaaaatcagctgccccacaaaattcagtacatttcctggagccccgtcggccacaagctggcttacgtctaccagaacaatatctacctcaaacaaagccccagagaggccccaattaaactcacctccgacggcaaagaaaatgaaatctttaacggaatccctgattgggtgtacgaagaggaaatgctggctaccaaatacgccctctggtggagcccaagcgggaaatacctcgcctacgtgcagtttaacgactccgatattccagtgattgagtattcctacttcggagaggaccagtaccccagaaaaattatcattccatatcctaaggccggagctaagaaccctaccgtgaaagtgttcatcgtcgacactacaaacatcgaagcctttggccctaaagaagtccctgtgccagccgtgatcgcttccagcgaccactatttcacctggctgacctgggtgaccgactccagagtcggcgtgcaatggctgaagaggattcagaacttctccgtcctggccatctgcgacttcaaagaaaacagcaacacatgggactgtcccgaaaaccagcaacatattgaagagtcccaaacaggctgggctggagggtttttcgtctccgccccatattttacatccgatggcagctcctattacaagatcttcagcgacaagaacgggtataaacacatccactacatcaatggcagcgtcgaaaacgccatccagattaccagcggcgaatgggaagccatttacattttcagagtgaccaacgatgccattttctatagctccaacgaatttgaggggtatcccggcaggagaaacatctacaaaattagcatcggatccaagccaattagaaagctgtgcatcacttgtaatctcaggaaggaaaggtgtcagtattacaccgccagattcagcgagaggagcaagtattacgccctgatctgctacgggcctggcattcctatcagcaccctgtttgagacagaaagcgacagagaactcagaatcctggaagacaaccaggagctccagtccgccctgcaagagatcattctgcccaaggaagagattaacaaactcgaggtggacggaatcaccctgtggtataagatgctgattccccctcaattcgataggagcaaaaagtacccactgctcatccaggtgtatgggggcccatgctcccagaatgtcaagcacacattcagcatcagctggattacatacctggccagcaaagaagggattatcgtcgccctggtggacggaagaggcaccgcctaccagggagataagatcctgcacgccgtctatagaaggctcggggtgtacgaagtcgaggaccaaatttccgccgtgaagaaattcattgagatgggcttcattgatgaaaagagaatcgctatctggggctgggcttatgggggctatgtcacctccctggccctgggcagcgggtccggcgtgttcaaatgcggcatcgctgtggcccccgtgtccagctgggagtactatgccagcatctatactgaaaggtttatgggcctccccgtggagagcgataacctggaacactataaaaacagcacagtgatggctagagccaaaaacttccaaaatgtggaatacctcctgatccacgggactgccgatgacaacgtccacttccaaaatagcgcccagatcgccaaggccctggtcaacgctcaggtggactttcaagccatgtggtacacagatcagaaccacggcattcccgggctcagctccaagcatctctatacccacatgactcacttcctgaagcagtgcttcagcctgagcgaataaa

SEQ ID NO: 17 - DNA, 2256 bp, unidentified
  misc_feature 1..2256: note = Codon pair optimized chicken FAP
  source 1..2256: mol_type = unassigned DNA
atggactggacctggatcctcttcctggtggctgctgccacccgggtgcacagcctgctgcccagcaaggtggtgaccaccgtggatggcccccgggccctgaccctggatgactacctgaatggcaacttccagtacaagacctacttcccctactgggtgtctgacagtgagtacctgcaccagaaccaggaggatgacatcatcctcttcaacgtggacatgaactacctgaccaccatcatgaccaacagcaccatgaagcaggtgaatgccagcaactacgtgatgagcagtgacaagtacttcatcgccctggagagcaactacagcaagctgtggagatacagctacacagccagctaccacatctatgacctcatctatggaggctttgtgacagagaaccagctgccccacaagatccagtacatctcctggagccctgtgggccacaagctggcctacgtgtaccagaacaacatctacctgaagcagagcccccgggaggcccccatcaagctgaccagtgatggcaaggagaatgagatcttcaatggcatcccagactgggtgtatgaggaggagatgctggccaccaagtatgccctgtggtggagccccagcggcaagtacctggcctacgtgcagttcaatgacagtgacatccctgtgattgagtacagctactttggagaggaccagtaccccaggaagatcatcatcccctaccccaaggccggggccaagaaccccaccgtgaaggtgttcatcgtggacaccaccaacatcgaggcctttggccccaaggaggtgcctgtgcctgctgtgattgccagcagtgaccactacttcacctggctgacctgggtgacagacagccgggtgggcgtgcagtggctgaagaggatccagaacttctctgtgctggccatctgtgacttcaaggagaacagcaacacctgggactgcccagagaaccagcagcacatcgaggagagccagacaggctgggccgggggcttctttgtgtctgccccctacttcaccagtgatggcagcagctactacaagatcttcagtgacaagaatggctacaagcacatccactacatcaatggctctgtggagaatgccatccagatcaccagcggggagtgggaggccatctacatcttccgggtgaccaatgatgccatcttctacagcagcaatgaatttgagggctacccaggaagaagaaacatctacaagatcagcattggcagcaagcccatccggaagctgtgcatcacctgcaacctgaggaaggagcgctgccagtactacacagcccgcttctctgagaggagcaagtactatgccctgatctgctatggccccggcatccccatcagcaccctgtttgagacagagagtgaccgggagctgcgcatcctggaggacaaccaggagctgcagagcgccctgcaggagatcatcctgcccaaggaggagatcaacaagctggaggtggatggcatcaccctgtggtacaagatgctgatccccccccagtttgaccgcagcaagaagtaccccctgctcatccaggtgtatggaggcccctgcagccagaatgtgaagcacaccttcagcatctcctggatcacctacctggccagcaaggagggcatcatcgtggccctggtggatggccggggcacagcctaccagggggacaagatcctgcacgccgtgtaccggcggctgggcgtgtatgaggtggaggaccagatctctgctgtgaagaagttcatcgagatgggcttcattgatgagaagagaattgccatctggggctgggcctatggaggctacgtgaccagcctggccctgggcagcggcagcggcgtgttcaagtgtggcattgctgtggcccctgtgtcctcctgggagtactatgccagcatctacacagagcgcttcatgggcctgcctgtggagagtgacaacctggagcactacaagaacagcaccgtgatggcccgggccaagaacttccagaatgtggagtacctgctcatccacggcacagcagatgacaacgtgcacttccagaacagcgcccagattgccaaggccctggtgaatgcccaggtggacttccaggccatgtggtacacagaccagaaccacggcatccccggcctgagcagcaagcacctgtacacccacatgacccacttcctgaagcagtgcttcagcctgtctgag

SEQ ID NO: 18 - DNA, 2263 bp, synthetic construct
  misc_feature 1..2263: note = Consensus FAP
  source 1..2263: mol_type = other DNA
atggattggacctggattctctttctggtcgccgctgccactagagtgcacagcctcaggcctagcagggtgcacaacagcgaagggaacaccactagggccctcaccctgaaggacatcctgaatgggacattcagctacaagaccttctttcctaattggatcagcggccaggagtatctccaccaatccaccgacaataacatcgtgctctacaatattgagaccggggaatcctataccattctcagcaactccacaatgaaatccgtgaacgccagcaattacggactgtcccccgacaggcaattcgcttatctggagtccgactactccaagctgtggaggtatagctatactgccacttaccacatttatgatctgtccaatggcgaatttgtgagagggaacgagctgcccagaccaatccagtacctgtgctggagcccagtggggagcaagctcgcttacgtctaccagaacaatatttatctgaaacagagacccgaagacccaccctttcagatcacctataatggcagggagaacaagatttttaacggcatccccgactgggtgtacgaagaggaaatgctggccaccaaatacgccctctggtggtcccctaacggcaaattcctggcttatgccgagttcaacgataccgacatccctgtcatcgcctatagctattacggagacgagcagtaccctagaaccatcaatattccctacccaaaagccggcgccaaaaaccccgtcgtgaggatctttattatcgacaccacatacccagagcacgtgggacccagggaggtccccgtgcctgctatgattgcctccagcgactactatttttcctggctcacctgggtgaccgacgaaagggtgtgcctgcagtggctgaaaaggatccagaacgtgtccgtgctcagcatctgcgatttcagggaggattggcagacctgggattgtcctaagacacaggaacacattgaagagagcagaacaggctgggccggcggattctttgtgtccacccccgtgttttcctatgacgctattagctattacaaaatcttctccgataaggatggctataaacatattcactacatcaaggacactgtggagaatgccattcagattactagcggcaagtgggaggccattaacatcttcagagtcacccaggatagcctgttctactccagcaacgagtttgagggataccccggcaggagaaacatctacaggattagcatcggcagctatcccccatccaagaaatgcatcacatgtcatctgaggaaggagagatgccagtactatacagccagcttctccgactatgctaaatattacgccctggtgtgttatggccctggcctcccaatctccactctgcatgacgggagaactgaccaagagatcaagattctggaggaaaacaaggagctggaaaacgctctgaagaatatccaactgcccaaggaagagatcaagaaactggaagtggatgaaattaccctgtggtacaaaatgatcctgcctccccaattcgatagatccaaaaagtaccccctcctgatccaagtgtacgggggcccttgcagccagtccgtgaggtccgtgtttgccgtcaactggatctcctacctggccagcaaggaagggatcgtgattgccctggtggatggcagaggcacagctttccagggagataaactcctgtacgccgtctatagaaaactcggcgtgtacgaggtggaggaccagattaccgccgtgaggaagtttatcgagatgggcttcatcgatgagaaaaggatcgccatttgggggtgggcttacggagggtacgtgtccagcctcgccctggccagcggaacagggctgttcaagtgcgggatcgccgtcgcccctgtgagctcctgggaatattacgcctccatctacaccgaaaggttcatgggcctgcccactaaggatgacaatctggagcactacaagaactccactgtcatggccagggctgagtacttcagaaatgtggactacctcctgatccatggcacagctgacgataacgtgcattttcagaacagcgcccagattgccaaagccctggtgaatgcccaggtggatttccaggccatgtggtatagcgaccagaaccacggaatcagcggcctcagcaccaaacacctgtatacccatatgacccacttcctgaagcagtgtttttccctgagcgattaaa

SEQ ID NO: 19 - DNA, 2259 bp, synthetic construct
  misc_feature 1..2259: note = Codon pair optimized consensus FAP
  source 1..2259: mol_type = other DNA
atggactggacctggatcctcttcctggtggctgctgccacccgggtgcacagcctgcggcccagccgggtgcacaacagcgagggcaacaccacccgggccctgaccctgaaggacatcctgaatggcaccttctcctacaagaccttcttccccaactggatcagcggccaggagtacctgcaccagagcacagacaacaacatcgtgctgtacaacattgaaacaggagagagctacaccatcctgagcaacagcaccatgaagtctgtgaatgccagcaactatggcctgtccccagacaggcagtttgcctacctggagagtgactacagcaagctgtggagatacagctacacagccacctaccacatctatgacctgagcaatggagagtttgtgagaggaaatgagctgccccggcccatccagtacctgtgctggagccctgtgggcagcaagctggcctacgtgtaccagaacaacatctacctgaagcagaggcctgaggacccccccttccagatcacctacaatggccgggagaacaagatcttcaatggcatcccagactgggtgtatgaggaggagatgctggccaccaagtatgccctgtggtggagccccaatggcaagttcctggcctatgccgagttcaatgacacagacatccctgtgattgcctacagctactatggagatgagcagtaccccaggaccatcaacatcccctaccccaaggccggggccaagaaccctgtggtgcgcatcttcatcattgacaccacctacccagagcacgtgggcccccgggaggtgcctgtgcctgccatgattgccagcagtgactactacttctcctggctgacctgggtgacagatgagcgggtgtgcctgcagtggctgaagaggatccagaatgtgtctgtgctgagcatctgtgacttccgggaggactggcagacctgggactgccccaagacccaggagcacatcgaggagagcagaacaggctgggccgggggcttctttgtgtccacccctgtgttctcctatgatgccatcagctactacaagatcttcagtgacaaggatggctacaagcacatccactacatcaaggacaccgtggagaatgccatccagatcaccagcggcaagtgggaggccatcaacatcttccgggtgacccaggacagcctcttctacagcagcaatgaatttgagggctacccaggaagaagaaacatctaccgcatcagcattggcagctacccccccagcaagaagtgcatcacctgccacctgaggaaggagcgctgccagtactacacagcctccttcagtgactatgccaagtactatgccctggtgtgctatggccccggcctgcccatcagcaccctgcatgatggaagaacagaccaggagatcaagatcctggaggagaacaaggagctggagaatgccctgaagaacatccagctgcccaaggaggagatcaagaagctggaggtggatgagatcaccctgtggtacaagatgatcctgcccccccagtttgaccgcagcaagaagtaccccctgctcatccaggtgtatggaggcccctgcagccagtctgtgcgcagcgtgtttgctgtgaactggatcagctacctggccagcaaggagggcatcgtgattgccctggtggatggccggggcacagccttccagggggacaagctgctgtatgctgtgtacaggaagctgggcgtgtatgaggtggaggaccagatcacagctgtgaggaagttcatcgagatgggcttcattgatgagaagagaattgccatctggggctgggcctatggaggctacgtgtccagcctggccctggccagcggcaccggcctcttcaagtgtggcattgctgtggcccctgtgtcctcctgggagtactatgccagcatctacacagagcgcttcatgggcctgcccaccaaggatgacaacctggagcactacaagaacagcaccgtgatggcccgggccgagtacttcagaaatgtggactacctgctcatccacggcacagcagatgacaacgtgcacttccagaacagcgcccagattgccaaggccctggtgaatgcccaggtggacttccaggccatgtggtacagtgaccagaaccacggcatcagcggcctgagcaccaagcacctgtacacccacatgacccacttcctgaagcagtgcttcagcctgagtgac

(19) United States
(12) Patent Application Publication
(10) Pub. No.: US 2025/0073331 A1
(43) Pub. Date: Mar. 6, 2025

(54) USE OF CODON DEOPTIMISATION AND OPTIMISATION TO PRODUCE A LARYNGOTRACHEITIS VIRUS-ATTENUATED VACCINE

(71) Applicant: EDGE ANIMAL HEALTH, INC., Boston, MA (US)

(72) Inventors: Kristi Mae MOORE, Shawnee, KS (US); Richard E. PETERSON, JR., Ijamsville, MD (US); David MOODY, Boston, MA (US)

(73) Assignee: EDGE ANIMAL HEALTH, INC., Boston, MA (US)

(21) Appl. No.: 18/528,294

(22) Filed: Dec. 4, 2023

Related U.S. Application Data
(60) Provisional application No. 63/579,703, filed on Aug. 30, 2023.

(51) Int. Cl. A61K 39/245; C07K 14/005; C12N 7/00; C12N 15/86

(52) U.S. Cl. CPC A61K 39/245; C07K 14/005; C12N 7/00; C12N 15/86; A61K 2039/5256; A61K 2039/54; A61K 2039/552; C12N 2710/16334; C12N 2710/16343; C12N 2760/18122; C12N 2760/18134

(57) ABSTRACT

An improved method of deoptimization of nucleic acids, particularly nucleic acids associated with genes and/or open reading frames (ORFs) of a variety of pathogens including viruses, retroviruses, bacteria, fungi, and the like. Nucleic acids may be related to genes and/or ORFs from infectious viruses or other diseases and be associated with the elicitation of a protective response when inserted, using recombinant techniques, into a vector and used in a vaccine composition. The nucleic acid sequence of one or more ORFs may be optimized or deoptimized for use in an improved recombinant vaccine to elicit an immune response against an infectious agent when administered to a subject using a variety of dosing and timing regimens.

### CROSS REFERENCE TO RELATED APPLICATIONS

This application claims benefit of and priority to U.S. Provisional Application No. 63/401,821, filed on Aug. 29, 2022, and to U.S. Provisional Application No. 63/579,703 filed on Aug. 30, 2023, each of which is incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 4, 2023, is named “067443.007US1.xml” and is 70,680 bytes in size. The sequence listing contained in this .XML file is part of the specification and is hereby incorporated by reference herein in its entirety.

### TECHNICAL FIELD

This disclosure generally relates to the field of vaccines, and more particularly to making and using attenuated vaccines.

### BACKGROUND

Laryngotracheitis virus is a cuboidal, enveloped DNA herpes virus affecting poultry (e.g. chickens, pheasants) (“Infectious laryngotracheitis (ILT, LT, Trach),” Poultry World (2021)). This disease is caused by Gallid alpha herpesvirus type 1 (GaHV-1), commonly known as infectious laryngotracheitis virus (ILTV) (Garcia, M., “Infectious Laryngotracheitis in Poultry,” Merck Manual, (2020)). The virus has an incubation period of 6-12 days. Different pathotypes have differing pathogenicity. Older carrier birds are a common source of infection. In broilers, the vast majority of outbreaks occur in flocks over 45 days of age, but the disease has also been diagnosed in flocks of a younger age. Aerosols, fomites, and ingestion of contaminated litter are common means for viral spread. Morbidity may range from 90% up to 100%, while mortality may range from 5% up to 70% (with an average of 10% up to 20%). Mortality is usually due to occlusion of the trachea by hemorrhage or exudate. In egg producing poultry, infection results in a drop in egg production (10-20%). Strains of low virulence produce little or no mortality, with mild respiratory signs and a slight decrease in egg production. Early signs of ILTV include watery eyes, nasal discharge, gasping, coughing bloody mucoid exudate, tracheal rales, and stretching necks. Most birds recover in 10-14 days if infection is not complicated by immunosuppression or a secondary bacterial or mycoplasma infection. After recovery, birds remain carriers for life and become a source of infection for other susceptible birds. The latent virus can be reactivated under stressful conditions. Infection also may be spread mechanically. Several epidemics have been traced to the transport of infected birds or contaminated equipment and litter. Laboratory tests used to diagnose ILTV include microscopic observation of intranuclear inclusion bodies in the lesions (usually trachea) and polymerase chain reaction (PCR).

Methods to prevent flock infections of ILTV include vaccinations. Vaccination is done with live attenuated vaccines and viral vector recombinant vaccines. Currently, there are two types of attenuated vaccines used to control the virus. The first type, the chicken embryo origin (CEO) vaccine, works well in the face of an outbreak, but can revert to virulence quickly and is often associated with mild disease (vaccinal LT). However, the CEO vaccines stimulate a robust immunity which suppresses shedding of field virus in vaccinated flocks, which is a valuable feature used to quickly control outbreaks. The second type, the tissue culture origin (TCO) vaccine, is a milder vaccine and safer to use, but it does not induce a strong immune response and thus is often insufficient for controlling an outbreak of the disease. Both of the current attenuated LT vaccines are administered to chickens 3-weeks old or older, usually through drinking water or by spray administration.

Traditionally, live vaccines have originated from virulent isolates that were attenuated by consecutive passages (i.e. serial passage) in embryos or tissue culture (Conrad, S. J., et al., “Attenuation of Marek's disease virus by codon pair deoptimization of a core gene,” Virology, 516: 219-226 (2018)). To design vaccines, field isolates and vaccine strains of ILTV have been differentiated by PCR amplification of single or multiple ILTV genome areas, followed by sequencing of the PCR products and analysis of the sequences obtained. More recently, field isolates and vaccines strains have been differentiated by full genome sequencing analysis. Genotyping of the virus is optional in the diagnosis of ILTV. Genotyping analysis answers whether the virus originated from previously used live attenuated vaccines, if it is related to previous outbreak strains, or if it is a new field strain.

Inactivated (killed) vaccines are made by growing more-or-less wild type virus and then inactivating it, for instance, with formaldehyde. A great deal of experimentation is required to find an inactivation treatment that kills all of the virus and yet does not damage the immunogenicity of the particle. In addition, residual safety issues remain in that the facility for growing the virus may allow virulent virus to escape or the inactivation may fail.

An attenuated live vaccine comprises a virus that has been subjected to mutations rendering it less virulent and usable for immunization. Live, attenuated viruses have many advantages as vaccines: they are often easy, fast, and cheap to manufacture; they are often easy to administer; and sometimes the residual growth of the attenuated virus allows “herd” immunization (immunization of subjects in close contact with the primary subject). These advantages are particularly important in an emergency, when a vaccine is rapidly needed. The major drawback of an attenuated vaccine is that it has some significant frequency of reversion to wild type virulence.

Other deoptimization approaches exist in the art outside of serial passage (Coleman, J. R., et al., “Virus attenuation by genome-scale changes in codon pair bias”, Science, 320: 1784-1787 (2008); Huang, Y., et al., “Codon pair optimization (CPO): a software tool for synthetic gene design based on codon pair bias to improve the expression of recombinant proteins in Pichia pastoris”, Microbial Cell Factories, 20: 209 (2021)) that attempt to use more analytical approaches to deoptimzation. However, these approaches face many flaws and more efficient deoptimization techniques are needed.

### INCORPORATION BY REFERENCE

Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the disclosure.

Citation or identification of any document in this application is not an admission that such document is available as prior art to the present disclosure.

### SUMMARY

It is to be understood that this summary is not an extensive overview of the disclosure. This summary is an example and not restrictive and it is intended to neither identify key or critical elements of the disclosure nor delineate the scope thereof. The sole purpose of this summary is to explain and exemplify certain concepts of the disclosure as an introduction to the following complete and extensive detailed description.

The present disclosure relates to deoptimized nucleic acid sequences of viruses, bacteria, protozoa, fungi, and other known pathogens. Deoptimized sequences may be incorporated into recombinant viral vectors. The present disclosure further relates to proteins, polypeptides, fusion proteins, fragments, and any combinations thereof encoded by deoptimized nucleic acid sequences of viruses. Deoptimized nucleic acids and resultant products (e.g. proteins) can be used in pharmaceutical compositions for immunizing avian subjects against a number of pathogens. Pathogens include but are not limited to Infectious Laryngotracheitis Virus (ILTV), Newcastle disease, Marek's disease, Bursal disease, and other pathogens affecting avians.

In an embodiment, the present disclosure relates to pharmaceutical compositions comprising a nucleic acid sequence of at least 80%, 90%, 95%, or 100% of SEQ ID NO:3. The present disclosure further relates to a pharmaceutical composition comprising products (e.g. proteins) of a nucleic acid sequence of at least 80%, 90%, 95%, or 100% of SEQ ID NO:3.

In an embodiment, the present disclosure relates to pharmaceutical compositions comprising a nucleic acid sequence of at least 80%, 90%, 95%, or 100% of SEQ ID NO:5. The present disclosure further relates to a pharmaceutical composition comprising products (e.g. proteins) of a nucleic acid sequence of at least 80%, 90%, 95%, or 100% of SEQ ID NO:5.

In an embodiment, the present disclosure relates to pharmaceutical compositions comprising a nucleic acid sequence of at least 80%, 90%, 95%, or 100% of SEQ ID NO:7. The present disclosure further relates to a pharmaceutical composition comprising products (e.g. proteins) of a nucleic acid sequence of at least 80%, 90%, 95%, or 100% of SEQ ID NO:7.

In an embodiment, the present disclosure relates to pharmaceutical compositions comprising a nucleic acid sequence of at least 80%, 90%, 95%, or 100% of SEQ ID NO:9. The present disclosure further relates to a pharmaceutical composition comprising products (e.g. proteins) of a nucleic acid sequence of at least 80%, 90%, 95%, or 100% of SEQ ID NO:9.

In an embodiment, the present disclosure relates to pharmaceutical compositions comprising a nucleic acid sequence of at least 80%, 90%, 95%, or 100% of SEQ ID NO:11. The present disclosure further relates to a pharmaceutical composition comprising products (e.g. proteins) of a nucleic acid sequence of at least 80%, 90%, 95%, or 100% of SEQ ID NO:11.

The present disclosure relates to methods of treating or preventing ILTV in avian subjects in need thereof including administering compositions disclosed herein to avian subjects. Administration can occur according to a variety of dosing and timing regimens and techniques disclosed herein. As non-limiting examples, pharmaceutical compositions may be administered by vaccine via injection (e.g., intramuscular, intravenous, etc.), infusion, eye drops, sprinkler systems, feedstocks and the like. Administration may also be performed in ovo after at least two weeks have passed since embryonation day. Administration may be performed more than once at varying doses and times.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 displays an example method according to the present disclosure.

FIG. 2 displays an example of a reversion based repair according to the present disclosure.

FIG. 3 displays an example of a large repeat repair according to the present disclosure.

FIG. 4 displays an example of a hairpin repair according to the present disclosure.

FIG. 5 displays results of an example according to the present disclosure.

FIG. 6 displays results of an example according to the present disclosure including normalized gLUC activity 24 hours post transfection.

FIG. 7 displays results of an example according to the present disclosure including normalized gLUC activity 6 hours post media change and 30 hours post transfection.

FIG. 8 displays results of an example according to the present disclosure including normalized gLUC activity 24 hours post media change and 48 hours post transfection.

FIG. 9 displays results of an example according to the present disclosure including normalized gLUC activity 6 hours post addition of ligand and 30 hours post transfection.

FIG. 10 displays results of an example according to the present disclosure including normalized gLUC activity 24 hours post addition of ligand and 48 hours post transfection

### DETAILED DESCRIPTION

The present disclosure can be understood more readily by reference to the following detailed description, examples, drawings, and claims, and their previous and following description. However, before the present compositions and/or methods are disclosed and described, it is to be understood that this disclosure is not limited to the specific compositions and/or methods disclosed unless otherwise specified, as such can, of course, vary. It is also to be understood that the terminology used herein is for the purpose of describing particular aspects only and is not intended to be limiting.

### I. Definitions

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present disclosure. All publications mentioned are incorporated herein by reference in their entirety.

Unless defined otherwise, all composition percentage values used herein are given in terms of weight percentage.

The use of the terms “a,” “an,” “the,” and similar referents in the context of describing the presently claimed disclosure (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or example language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

As used herein, an “adjuvant” is a substance that is able to favor or amplify the cascade of immunological events, ultimately leading to a better immunological response, i.e., the integrated bodily response to an antigen. An adjuvant is in general not required for the immunological response to occur, but favors or amplifies this response. Example adjuvants include, but are not limited to, Freund's Incomplete Adjuvant (IFA), Freund's complete adjuvant, B30-MDP, LA-15-PH, montanide, saponin, aluminum salts such as aluminum hydroxide (Amphogel, Wyeth Laboratories, Madison, N.J.), alum, lipids, keyhole limpet protein, hemocyanin, the MF59 microemulsion, a mycobacterial antigen, vitamin E, non-ionic block polymers, muramyl dipeptides, polyanions, amphipatic substances, ISCOMs (immune stimulating complexes, such as those disclosed in European Patent EP 109942), vegetable oil, Carbopol, aluminium oxide, oil-emulsions (such as Bayol F or Marcol 52), E. coli heat-labile toxin (LT), Cholera toxin (CT), and combinations thereof.

As used herein, “administration” as it applies to a human, primate, mammal, mammalian subject, animal, poultry, veterinary subject, placebo subject, research subject, experimental subject, cell, tissue, organ, or biological fluid, refers to contact of an exogenous ligand, reagent, placebo, small molecule, pharmaceutical agent, therapeutic agent, diagnostic agent, or composition to the subject, cell, tissue, organ, or biological fluid, and the like. “Administration” can refer to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

As used herein, the term “aids in the protection” does not require complete protection from any indication of infection. For example, “aids in the protection” can mean that the protection is sufficient such that, after challenge, symptoms of the underlying infection are at least reduced, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection.

As used herein, an “analog” or “derivative” with reference to a peptide, polypeptide or protein refers to another peptide, polypeptide or protein that possesses a similar or identical function as the original peptide, polypeptide or protein, but does not necessarily comprise a similar or identical amino acid sequence or structure of the original peptide, polypeptide or protein. An analog preferably satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the original amino acid sequence (b) a proteinaceous agent encoded by a nucleotide sequence that hybridizes under stringent conditions to a nucleotide sequence encoding the original amino acid sequence; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the original amino acid sequence.

As used herein, “antibody” refers to a peptide or polypeptide derived from, modeled after, or substantially encoded by an immunoglobulin gene or immunoglobulin genes, or fragments thereof, capable of specifically binding an antigen or epitope (Wilson, J. Immunol. Methods, 1994; Yarmush, J. Biochem. Biophys, 1992). The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward, et al., Nature, 1989), which consists of a VH domain; and (vi) an isolated complementarity determining region (CDR). Single chain antibodies are also included by reference in the term “antibody.”

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, “antigen presenting cells” (APCs) are cells of the immune system used for presenting antigen to T cells. APCs include dendritic cells, monocytes, macrophages, marginal zone Kupffer cells, microglia, Langerhans cells, T cells, and B cells. Dendritic cells occur in at least two lineages. The first lineage encompasses pre-DC1, myeloid DC1, and mature DC1. The second lineage encompasses CD34⁺CD45RA⁻ early progenitor multipotent cells, CD34⁺CD45RA⁺ cells, CD34⁺CD45RA⁺CD4⁺ IL-3Ra pro-DC2 cells, CD4⁺CD11c⁻ plasmacytoid pre-DC2 cells, lymphoid human DC2 plasmacytoid-derived DC2s, and mature DC2s.

As used herein, an “attenuated pathogen” or a “deoptimized pathogen” is a pathogen with a decreased or weakened ability to produce disease while retaining the ability to stimulate an immune response like that of the natural pathogen. In one example, a live pathogen is attenuated by deoptimizing one or more codons in one or more genes, such as an immunogenic surface antigen or a housekeeping gene. In another example, a pathogen is attenuated by selecting for avirulent variants under certain growth conditions (for example see Sabin and Boulger. J. Biol. Stand. 1:115-8; 1973; Sutter et al., 2003. Poliovirus vaccine—live, p. 651-705. In S. A. Plotkin and W. A. Orenstein (ed.), Vaccines, Fourth ed. W.B. Saunders Company, Philadelphia). A “deoptimized pathogen” further refers to a pathogen having a nucleic acid coding sequence with one or more deoptimized codons. In some examples, refers to the isolated deoptimized nucleic acid sequence itself, independent of the pathogenic organism

As used herein, the term “avian” includes chicken, turkeys, ducks, game birds, including but not limited to, quail, pheasants, and geese, and ratites including but not limited to ostrich and emu. The term “poultry” denotes birds of the order Galliformes such as, for example, ordinary domestic fowl.

As used herein, “attenuated gene” encompasses a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, and frameshift mutations in regulatory regions of the gene, coding regions of the gene, non-coding regions of the gene, or any combination thereof.

A nucleic acid “coding sequence” or a “sequence encoding” a particular protein or peptide, is a nucleotide sequence which is transcribed and translated into a polypeptide in vitro or in vivo when placed under the control of appropriate regulatory elements.

The boundaries of the coding sequence are determined by a start codon at the 5′-terminus and a translation stop codon at the 3′-terminus. A coding sequence can include, but is not limited to, prokaryotic sequences, cDNA from eukaryotic mRNA, genomic DNA sequences from eukaryotic (e.g., avian) DNA, and even synthetic DNA sequences. A transcription termination sequence can be located 3′ to the coding sequence.

As used herein, a “codon” is a specific sequence of three adjacent nucleotide bases on a strand of DNA or RNA that provides genetic code information for a particular amino acid or a termination signal. Codons can be deoptimized, for example, by manipulating the nucleic acid sequence using molecular biology methods. Attenuated pathogens, such as an attenuated virus or bacterium, can be used in an immune composition to stimulate an immune response in a subject. For example, attenuated pathogens can be used in an attenuated vaccine to produce an immune response without causing the severe effects of the disease. Particular examples of attenuated vaccines include, but are not limited to, measles, mumps, rubella, polio, typhoid, yellow fever, and varicella vaccines.

As used herein, “codon pair bias” refers to a phenomenon wherein certain codons tend to be found next to each other while some codon pair combinations are avoided. Codon pair bias is ubiquitous among genetic systems (Ikemura, J. Mol. Biol. 146:1-21, 1981; Ikemura, J. Mol. Biol. 158:573-97, 1982). The strength and direction of codon usage bias is related to genomic G+C content and the relative abundance of different isoaccepting tRNAs (Akashi, Curr. Opin. Genet. Dev. 11:660-6, 2001; Duret, Curr. Opin. Genet. Dev. 12:640-9, 2002; Osawa et al., Microbiol. Rev. 56:229-64, 1992). Codon usage can affect the efficiency of gene expression. In Escherichia coli (Ikemura, J. Mol. Biol. 146:1-21, 1981; Xia Genetics 149:37-44, 1998), Saccharomyces cerevisiae (Bennetzen and Hall, J. Biol. Chem. 257:3026-31, 1982; Ikemura, J. Mol. Biol. 158:573-97, 1982), Caenorhabditis elegans (Duret, Curr. Opin. Genet. Dev. 12:640-9, 2002), Drosophila melanogaster (Moriyama and Powell, J. Mol. Evol. 45:514-23, 1997), and Arabidopsis thaliana (Chiapello et al. Gene 209:GC1-GC38, 1998) the most highly expressed genes use codons matched to the most abundant tRNAs (Akashi and Eyre-Walker, Curr. Opin. Genet. Dev. 8:688-93, 1998). By contrast and as a non-limiting example, in humans and other vertebrates, codon usage bias is more strongly correlated with the G+C content of the isochore where the gene is located (Musto et al., Mol. Biol. Evol. 18:1703-7, 2001; Urrutia and Hurst, Genetics 159:1191-9, 2001) than with the breadth or level of gene expression (Duret, Curr. Opin. Genet. Dev. 12:640-9, 2002) or the number of tRNA genes (Kanaya et al., J. Mol. Evol. 53:290-8, 2001).

As used herein, “codon pair score” indicates the likelihood of a codon pair occurring in a genome. A lower codon pair score (CPS) corresponds to a codon pair that is not seen as frequently as other codon pairs (i.e. a codon pair with a lower CPS is rarer).

Functionally equivalent amino acid residues often can be substituted for residues within the sequence resulting in a conservative amino acid substitution. Such alterations define the term “a conservative substitution” as used herein. For example, one or more amino acid residues within the sequence can be substituted by another amino acid of a similar polarity, which acts as a functional equivalent, resulting in a silent alteration. Substitutions for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred conservative substitutions are: Lys for Arg and vice versa such that a positive charge may be maintained; Glu for Asp and vice versa such that a negative charge may be maintained; Ser for Thr such that a free-OH can be maintained; and Gln for Asn such that a free NH2 can be maintained. The amino acids also can be placed in the following similarity groups: (1) proline, alanine, glycine, serine, and threonine; (2) glutamine, asparagine, glutamic acid, and aspartic acid; (3) histidine, lysine, and arginine; (4) cysteine; (5) valine, leucine, isoleucine, methionine; and (6) phenylalanine, tyrosine, and tryptophan.

In a related embodiment, two highly homologous DNA sequences can be identified by their own homology, or the homology of the amino acids they encode. Such comparison of the sequences can be performed using standard software available in sequence data banks. In a particular embodiment two highly homologous DNA sequences encode amino acid sequences having about 80% identity, more preferably about 90% identity and even more preferably about 95% identity. More particularly, two highly homologous amino acid sequences have about 80% identity, even more preferably about 90% identity and even more preferably about 95% identity.

As used herein, protein and DNA sequence percent identity can be determined using software such as MacVector v9, commercially available from Accelrys (Burlington, Mass.) and the Clustal W algorithm with the alignment default parameters, and default parameters for identity. See, e.g., Thompson, et al., 1994. Nucleic Acids Res. 22:4673-4680. ClustalW is freely downloadable for Dos, Macintosh and Unix platforms from, e.g., EMBLI, the European Bioinformatics Institute. These and other available programs can also be used to determine sequence similarity using the same or analogous default parameters.

As used herein, “deoptimization of a codon” is to replace a preferred codon in a nucleic acid sequence with a synonymous codon (one that codes for the same amino acid) less frequently used (unpreferred) in the organism. Each organism has a particular codon usage bias for each amino acid, which can be determined from publicly available codon usage tables (for example see Nakamura et al., Nucleic Acids Res. 28:292, 2000 and references cited therein; Sharp et al., Nucleic Acids Res. 16:8207-11, 1988; Chou and Zhang, AIDS Res. Hum. Retroviruses. December; 8(12):1967-76, 1992; West and Iglewski et al., Nucleic Acids Res. 16:9323-35, 1988, Rothberg and Wimmer, Nucleic Acids Res. 9:6221-9, 1981; Jenkins et al., J. Mol. Evol. 52:383-90, 2001; and Watterson, Mol. Biol. Evol. 9:666-77, 1992; all herein incorporated by reference). In addition, codon usage tables are available for several organisms on the internet at GenBank's website.

As used herein, “effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition.

As used herein an “expression cassette” is a recombinant nucleic acid that minimally comprises a promoter and a heterologous coding sequence operably linked to that promoter. In many such embodiments, the expression cassette further comprises a transcription terminator sequence.

As used herein, “fragments” in the context of polypeptides include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a larger polypeptide.

As used herein, a “global minimum” refers to the smallest value of an overall set. Whereas local minimums may be the smallest value in a nearby (i.e. local) section of an overall set, a global minimum is the lowest value of the total set.

As used herein, a “heterologous nucleotide sequence” is a nucleotide sequence that is added to a nucleotide sequence of the present disclosure by recombinant methods to form a nucleic acid that is not naturally formed in nature. Heterologous nucleotide sequences can also encode fusion (e.g., chimeric) proteins. In addition, a heterologous nucleotide sequence can encode peptides and/or proteins that contain regulatory and/or structural properties. In other such embodiments, a heterologous nucleotide sequence can encode a protein or peptide that functions as a means of detecting the protein or peptide encoded by the nucleotide sequence of the present disclosure after the recombinant nucleic acid is expressed. In still another embodiment, the heterologous nucleotide sequence can function as a means of detecting a nucleotide sequence of the present disclosure. A heterologous nucleotide sequence can comprise non-coding sequences including restriction sites, regulatory sites, promoters and the like.

As used herein an amino acid sequence is 100% “homologous” to a second amino acid sequence if the two amino acid sequences are identical, and/or differ only by neutral or conservative substitutions as defined below. Accordingly, an amino acid sequence is about 80% “homologous” to a second amino acid sequence if about 80% of the two amino acid sequences are identical, and/or differ only by neutral or conservative substitutions.

As used herein, an “immune cell” refers to any cell from the hemopoietic origin including, but not limited to, T cells, B cells, NK cell, monocytes, dendritic cells, and macrophages.

An “immunogenic agent” or “immunogen” is capable of inducing an immunological response against itself on administration to a mammal, optionally in conjunction with an adjuvant.

As used herein, the terms “immunologic,” “immunological” or “immune response” is the development of a beneficial humoral (antibody mediated) and/or a cellular (mediated by antigen-specific T cells or their secretion products) response directed against a peptide in a recipient patient. For example, the immune response by a desired amount, for example by at least 5%, at least 10%, at least 15%, at least 20%, at least 25%, at least 30%, at least 50%, at least 75%, or even at least 90%, as compared to an immune response in the absence of compositions described herein. This increase can result in decreasing or slowing the progression of, a disease or condition associated with a pathogenic infection. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, “maintaining” or “maintain” or “maintenance” refers to the ability to preserve the optimization or deoptimization of an optimized or deoptimized sequence during repairs. Repairs are needed when an optimized or deoptimized sequence has become too complex to be easily synthesized. Synthetic complexity is caused by the introduction of repeat sequences in an overall sequence during optimization or deoptimization. Repairs selectively revert codons in repeat sequences to reduce synthetic complexity so that an optimized or deoptimized sequence can be synthesized. As such, “maintaining” or “maintain” or “maintenance” refers to not lowering the level of optimization or deoptimization during repair. Optimization and deoptimization can be shown by codon pair bias (CPB), as described herein.

As used herein the term “parenteral administration” includes subcutaneous injections, submucosal injections, intravenous injections, intramuscular injections, intradermal injections, and infusion.

As used herein, “operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

As used herein, the terms “percent sequence identity” and “% sequence identity” refer to the percentage of sequence similarity found by a comparison or alignment of two or more amino acid or nucleic acid sequences. Percent identity can be determined by a direct comparison of the sequence information between two molecules by aligning the sequences, counting the exact number of matches between the two aligned sequences, dividing by the length of the shorter sequence, and multiplying the result by 100. An algorithm for calculating percent identity is the Smith-Waterman homology search algorithm (see, e.g., Kann and Goldstein (2002) Proteins 48:367-376; Arslan, et al. (2001) Bioinformatics 17:327-337).

As used herein, “peptide” refers to a short sequence of amino acids, where the amino acids are connected to each other by peptide bonds. A peptide may occur free or bound to another moiety, such as a macromolecule, lipid, oligo- or polysaccharide, and/or a polypeptide. Where a peptide is incorporated into a polypeptide chain, the term “peptide” may still be used to refer specifically to the short sequence of amino acids. A “peptide” may be connected to another moiety by way of a peptide bond or some other type of linkage. A peptide is at least two amino acids in length, wherein the maximal length is a function of custom or context.

As used herein, a “pharmaceutically acceptable excipient” or “diagnostically acceptable excipient” includes but is not limited to, sterile distilled water, saline, phosphate buffered solutions, amino acid based buffers, or bicarbonate buffered solutions. Remington's Pharmaceutical Sciences, by E. W. Martin, Mack Publishing Co., Easton, Pa., 15th Edition (1975), describes additional compositions and formulations suitable for pharmaceutical delivery of one or more therapeutic compounds or molecules, such as one or more nucleic acid molecules, proteins or immunogenic compositions disclosed herein. An excipient selected and the amount of excipient used will depend upon the mode of administration. Administration comprises an injection, infusion, or a combination thereof.

In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

As used herein the terms “polynucleotide”, or a “nucleic acid” or a “nucleic acid molecule” are used interchangeably and denote a deoxyribonucleotide or ribonucleotide polymer including but not limited to cDNA, mRNA, genomic DNA, genomic RNA, and synthetic (such as chemically synthesized) DNA. Includes nucleic acid sequences that have naturally-occurring, modified, or non-naturally-occurring nucleotides linked together by naturally-occurring or non-naturally-occurring nucleotide linkages. Nucleic acid molecules can be modified chemically or biochemically and can contain non-natural or derivatized nucleotide bases. Such modifications include, for example, labels, methylation, substitution of one or more of the naturally occurring nucleotides with analogs, and internucleotide linkage modifications.

Nucleic acid molecules can be in any topological conformation, including single-stranded, double-stranded, partially duplexed, triplexed, hairpinned, circular, linear, and padlocked conformations. Where single-stranded, a nucleic acid molecule can be the sense strand or the antisense strand. Also included are synthetic molecules that mimic polynucleotides in their ability to bind to a designated sequence via hydrogen bonding and other chemical interactions. Such molecules are known and include, for example, molecules in which peptide linkages are substituted for phosphate linkages in the backbone.

The disclosure includes isolated nucleic acid molecules that include specified lengths of a nucleotide sequence. Such molecules can include at least 10, at least 15, at least 20, at least 25, at least 30, at least 35, at least 40, at least 45, at least 50, at least 100, at least 300 or at least 500 nucleotides of these sequences or more, and can be obtained from any region of a nucleic acid molecule.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “prophylactic agent” refers to an agent that can be used in the prevention of a disorder or disease prior to the detection of any symptoms of such disorder or disease. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, “protein” generally refers to the sequence of amino acids comprising a polypeptide chain. Protein may also refer to a three-dimensional structure of the polypeptide. “Denatured protein” refers to a partially denatured polypeptide, having some residual three-dimensional structure or, alternatively, to an essentially random three dimensional structure, as is the case in a totally denatured protein. Polypeptide variants can be produced by glycosylation, phosphorylation, sulfation, disulfide bond formation, deamidation, isomerization, cleaving points in signal or leader sequence processing, covalent and non-covalently bound cofactors, oxidized variants, and the like.

As used herein, a “rare” or “rarer” codon is one of at least two synonymous codons encoding a particular amino acid that is present in an mRNA at a significantly lower frequency than the most frequently used codon for that amino acid. Thus, the rare codon may be present at about a 2-fold lower frequency than the most frequently used codon. In some examples, the rare codon is present at least a 3-fold, more preferably at least a 5-fold, lower frequency than the most frequently used codon for the amino acid. Conversely, a “frequent” codon is one of at least two synonymous codons encoding a particular amino acid that is present in an mRNA at a significantly higher frequency than the least frequently used codon for that amino acid. The frequent codon may be present at about a 2-fold, at least about a 3-fold, or at least about a 5-fold, higher frequency than the least frequently used codon for the amino acid.

As a non-limiting example, human genes use the leucine codon CTG 40% of the time, but use the synonymous CTA only 7% of the time. Thus, CTG is a frequent codon, whereas CTA is a rare codon. Roughly consistent with these frequencies of usage, there are 6 copies in the genome for the gene for the tRNA recognizing CTG, whereas there are only 2 copies of the gene for the tRNA recognizing CTA. Similarly, human genes use the frequent codons TCT and TCC for serine 18% and 22% of the time, respectively, but the rare codon TCG only 5% of the time. TCT and TCC are read, via wobble, by the same tRNA, which has 10 copies of its gene in the genome, while TCG is read by a tRNA with only 4 copies. It is well known that those mRNAs that are very actively translated are strongly biased to use only the most frequent codons. This includes genes for ribosomal proteins and glycolytic enzymes. On the other hand, mRNAs for relatively non-abundant proteins may use the rare codons.

As used herein, “recombinant” when used with reference to a nucleic acid, cell, animal, virus, plasmid, vector, or the like, indicates modification by the introduction of an exogenous, non-native nucleic acid, alteration of a native nucleic acid, or by derivation in whole or in part from a recombinant nucleic acid, cell, virus, plasmid, or vector. Recombinant protein refers to a produced or secreted protein derived from a recombinant nucleic acid, virus, plasmid, vector, or the like.

As used herein, the term “subject” refers to a human or non-human organism. Thus, the methods and compositions described herein are applicable to both human and veterinary disease. In certain embodiments, subjects are poultry. In certain embodiments, subjects are “patients,” such as living humans that are receiving medical care for a disease or condition. This includes persons with no defined illness who are being investigated for signs of pathology.

The term “substantially,” as used in the context of binding or exhibited effect, is intended to denote that the observed effect is physiologically or therapeutically relevant. Thus, for example, a molecule is able to substantially block an activity of a ligand or receptor if the extent of blockage is physiologically or therapeutically relevant (for example if such extent is greater than 60% complete, greater than 70% complete, greater than 75% complete, greater than 80% complete, greater than 85% complete, greater than 90% complete, greater than 95% complete, or greater than 97% complete). Similarly, a molecule is said to have substantially the same immunospecificity and/or characteristic as another molecule, if such immunospecificities and characteristics are greater than 60% identical, greater than 70% identical, greater than 75% identical, greater than 80% identical, greater than 85% identical, greater than 90% identical, greater than 95% identical, or greater than 97% identical).

As used herein, “systemic administration” is administration into the circulatory system of the body (comprising the cardiovascular and lymphatic system), thus affecting the body as a whole rather than a specific locus such as the gastro-intestinal tract (via e.g., oral or rectal administration) and the respiratory system (via e.g., intranasal administration). Systemic administration can be performed e.g., by administering into muscle tissue (intramuscular), into the dermis (intradermal or transdermal), underneath the skin (subcutaneous), underneath the mucosa (submucosal), in the veins (intravenous) etc.

As used herein, the term “therapeutically effective amount” is defined as an amount of a reagent or pharmaceutical composition that is sufficient to induce a desired immune response specific for encoded heterologous antigens to show a patient benefit (e.g. to cause a decrease, prevention, or amelioration of the symptoms of the condition being treated). When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual (U.S. Pat. No. 5,888,530).

As used herein, the term “transcription terminator sequence” is used interchangeably with the term “polyadenylation regulatory element” and is a sequence that is generally downstream from a DNA coding region and that may be required for the complete termination of the transcription of that DNA coding sequence.

As used herein, “treatment” or “treating” (with respect to a condition or a disease) is an approach for obtaining beneficial or desired results including and preferably clinical results. For purposes of this disclosure, beneficial or desired results with respect to a disease include, but are not limited to, one or more of improving a condition associated with a disease, curing a disease, lessening severity of a disease, delaying progression of a disease, alleviating one or more symptoms associated with a disease, increasing the quality of life of one suffering from a disease, and/or prolonging survival. Likewise, for purposes of this disclosure, beneficial or desired results with respect to a condition include, but are not limited to, one or more of improving a condition, curing a condition, lessening severity of a condition, delaying progression of a condition, alleviating one or more symptoms associated with a condition, increasing the quality of life of one suffering from a condition, and/or prolonging survival.

As used herein, a “vaccine” is a composition that is suitable for application to an animal (including, in certain embodiments, humans, while in other embodiments being specifically not for humans) comprising one or more antigens typically combined with a pharmaceutically acceptable carrier such as a liquid containing water, which upon administration to the animal induces an immune response strong enough to minimally aid in the protection from a clinical disease arising from an infection with a wild-type micro-organism, i.e., strong enough for aiding in the prevention of the clinical disease, and/or preventing, ameliorating or curing the clinical disease.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

### II. Infectious Laryngotracheitis Virus (ILTV)

Infectious laryngotracheitis (ILT) is an acute respiratory disease of chickens that causes significant economic losses to poultry industry worldwide (Bagust et al., 2000, Rev Sci Tech 19, 483-492; Bagust, 1986, Avian Pathol 15, 581-595). The causative pathogen, ILTV, is a member of the genus Iltovirus in the family Herpesviridae (Bagust et al., 2000, supra; Fuchs et al., 2007, Vet Res 38, 261-279). Currently, live attenuated vaccines are used to control ILT infections. However, the live-attenuated vaccines are not satisfactory since they can revert to virulence after bird-to-bird passage (Guy et al., 1991, Avian Dis 35, 348-355) and can induce latent infections (Hughes et al., 1991, Arch Virol 121, 213-218). Several alternative strategies have been used to develop improved ILTV vaccines (Mauricio et al., 2013, Avian Pathol 42, 195-205). One of the strategies has been the creation of ILTV deletion mutants for use as attenuated live-virus vaccines (Mauricio et al., 2013, supra). Two of the concerns of using gene deleted ILTV vaccine are the establishment of latency and the possibility that the gene-deleted vaccine virus could become virulent after recombination with different attenuated vaccine used in the same region (Sang-Won et al, 2012, Science 337, 188; Henderson et al., 1991, Am J Vet Res 52, 820-825). All studies conducted to date suggest that a virus-vectored ILTV vaccine will be most effective for prevention and control of TLT (Tong et al., 2001, Avian pathol 30, 143-148; Sun et al., 2008, Avian Dis 52, 111-117; Vagnozzi et al., 2012, Avian Pathol 41, 21-31). A vectored-vaccine will be safe and not lead to reversion to virulence or establishment of latency. However, current live virus vectored vaccines against TLT have limitations (Mauricio et al., 2013, supra; Vagnozzi et al. 2012, supra): (i) route of administration to large number of one-day old chicks, (ii) effective delivery of vaccine antigen to the mucosal surface, (iii) production cost, and (iv) incomplete protection. Therefore, there is a need to evaluate additional viral vectors to deliver ILTV antigens to chickens.

The ILTV gD gene appears to encode a glycoprotein of 434 amino acids in length having a molecular weight of 48,477 daltons, although others have suggested that a downstream start codon, which leads to an ILTV gD protein comprising only 377 amino acid residues, is the actual start codon [Wild et al., Virus Genes 12:104-116 (1996)]. The ILTV gI gene encodes a glycoprotein of 362 amino acids in length having a molecular weight of 39,753 daltons [U.S. Pat. No. 6,875,856, hereby incorporated by reference]. Nucleic acids encoding natural and/or laboratory derived variants of the ILTV gD and ILTV gI may be substituted for those presently exemplified.

### III. Method of Deoptimizing Infectious Laryngotracheitis Virus

The present disclosure relates to an improved method of deoptimizing viruses. The method can be applied to a wide variety of viruses and retroviruses known in the art. As non-limiting examples, methods of deoptimization disclosed herein can be applied to Newcastle disease, bursal disease, Marek's disease, ILTV, other viruses affecting poultry (e.g. Gallus gallus), and any fragments or fusion proteins thereof. In a particular method, the virus can be ILTV. The method can also be applied to nucleic acids of bacteria, fungi, and protozoa. Any virus or retrovirus that is intended to be used in a vaccine or other such prophylactic treatment or therapeutic treatments can be deoptimized using the method described herein. The deoptimization method can be applied to any of a variety of nucleic acids including but not limited to ribonucleic acid (RNA), double-stranded RNA (dsRNA), positive-strand RNA (psRNA), negative-strand RNA (nsRNA), deoxyribonucleic acid (DNA), single-stranded DNA (ssDNA), double-stranded DNA (dsDNA), and any combinations thereof.

In some examples, the method includes deoptimizing at least one codon in a coding sequence of the pathogen, thereby generating a deoptimized coding sequence. Such deoptimization can reduce virulence of a pathogen. In some examples, at least one codon can be deoptimized by being replaced with a synonymous codon. In some examples, at least one codon can be deoptimized by being replaced with a rarer codon. In some examples, more than one coding sequence of the pathogen is deoptimized, such as at least one, at least two, or at least 5 coding sequences, such as deoptimizing 1, 2, 3, 4, 5, 6, 7, 8, 9 or 10 coding sequences of the pathogen.

In some examples, viral pathogen sequences are deoptimized in one or more nucleic acid sequences that encode proteins encoding surface antigens which are determinants of immunity, such as a capsid sequences, or spike glycoproteins.

More than one codon in the one or more coding sequences can be deoptimized, such as at least 15 codons, at least 20 codons, at least 30 codons, at least 40 codons, at least 50 codons, at least 60 codons, at least 70 codons, at least 100 codons, at least 200 codons, at least 500 codons, or even at least 1000 codons, in each coding sequence. In some examples, at least 20% of the coding sequence of each desired gene is deoptimized, such as at least 30%, at least 40%, at least 50%, at least 60%, at least 70%, at least 80%, at least 90%, at least 95%, or even at least 97% deoptimized.

As a non-limiting example, deoptimizing a codon composition alters the G+C content of a coding sequence, such as increases or decreases the G+C content by at least 10%, for example increases the G+C content of a coding sequence by at least 10%, such as at least 20%, at least 30%, at least 40%, at least 50%, at least 60%, at least 70%, at least 80%, or even by at least 90%, or decreases the G+C content of a coding sequence by at least 10%, such as at least 20%, at least 30%, at least 40%, at least 50%, at least 60%, at least 70%, at least 80%, or even by at least 90%. However, the G+C content can be altered in combination with deoptimizing one or more codons in a pathogen sequence. For example, some of the nucleotide substitutions can be made to deoptimize codons (which may or may not alter the G+C content of the sequence), and other nucleotide substitutions can be made to alter the G+C content of the sequence (which may or may result in a deoptimized codon). Altering the G+C content of the sequence may also result in a deoptimized codon, but is not required in all instances.

As a non-limiting example, deoptimizing the codon composition results in an altered frequency (number) of CG dinucleotides, TA dinucleotides, or both, in a coding sequence. For example, deoptimization of one or more codons may increase or decrease the frequency of CG or TA dinucleotides in the sequence by at least 10%, for example increase the number of CG or TA dinucleotides in a coding sequence by at least 10%, such as at least 20%, at least 30%, at least 40%, at least 50%, at least 60%, at least 100%, at least 200%, or even by at least 300%, or decrease in the number of CG or TA dinucleotides in a coding sequence by at least 10%, such as at least 20%, at least 30%, at least 40%, at least 50%, at least 60%, at least 70%, at least 80%, or even by at least 90%. Whether the number of CG or TA dinucleotides is increased or decreased will depend on the sequence of the pathogen of interest.

In some examples, the amount of rare codons (as indicated by codon pair scores) may be altered in deoptimization. In such examples, the amount of rare codons in a sequence may be increased. The amount of rare codons in a sequence may be increased by at least 10%, such as at least 20%, at least 30%, at least 40%, at least 50%, at least 60%, at least 70%, at least 80%, or even by at least 90%, or decreases the G+C content of a coding sequence by at least 10%, such as at least 20%, at least 30%, at least 40%, at least 50%, at least 60%, at least 70%, at least 80%, or even by at least 90% during deoptimization.

The present disclosure relates to reducing virulence of a pathogen by any amount sufficient to attenuate the pathogen. In some examples, virulence of the deoptimized pathogen is reduced by at least 20%, such as at least 30%, at least 40%, at least 48%, at least 50%, at least 75%, at least 80%, at least 90%, at least 95%, or even at least 97%, as compared to virulence of a pathogen (of the same species and strain) having a coding sequence that has not undergone deoptimization, as described herein.

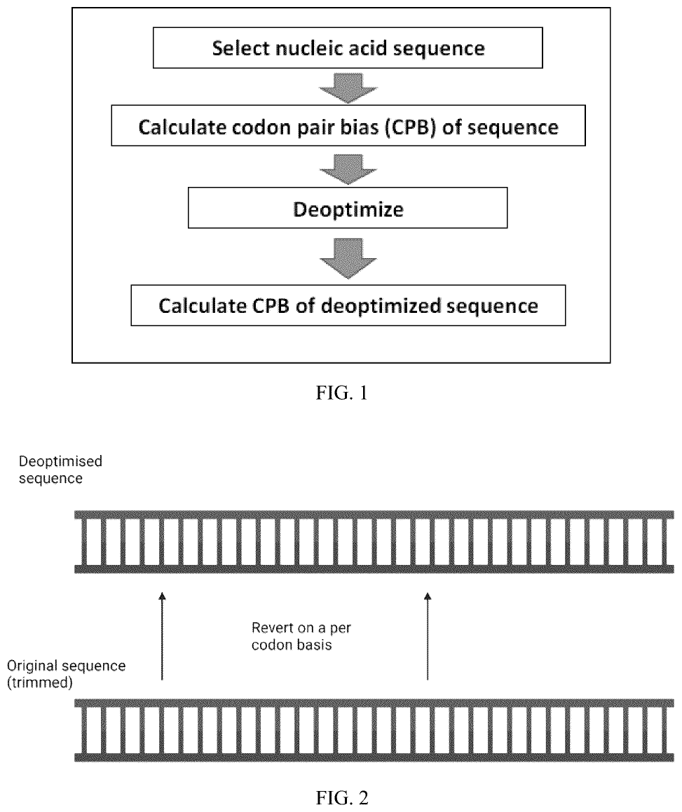

The present disclosure relates to a deoptimization method including codon deoptimization, as shown in FIG. 1. More specifically, the present disclosure relates to a method to produce a recombinant virus by recoding certain viral genes to employ synonymous but rarely-used codon pairs that will result in viral attenuation. The deoptimized viral variant is useful as an attenuated vaccine to elicit an immune response in a subject infected while showing a decreased virulence following vaccination. The present disclosure uses an optimized and efficient deoptimization method to analytically identify a global minimum codon pair bias (CPB), which indicates a total usage of rarer codon pairings. Codon pair bias is tied to codon pair score (CPS), which indicates a rarer codon pairing. The general relation between is defined as follows:

$$CPB = \frac{1}{n} {\sum }_{i = 1}^{n} {CPS}_{i} \\ where$$

$$CPS = ln ( \frac{C 1 C 2}{( \frac{C 1 X C 2}{A 1 X A 2} ) X A 1 A 2} )$$

where
C1=codon count 1
C2=codon count 2
C1C2=codon pair count
A1=amino acid count 1
A2=amino acid count 2
A1A2=amino acid pair count

By designing a deoptimized, recombinant virus with a globally minimized CPB value, the method of attenuating viral strains for live vaccine administration is greatly improved. In such manner, immunogenicity is achieved in an efficient enough manner to reduce or treat a virus and viral symptoms while preventing unwanted virulence. Previous attempts to achieve the goal of immunogenicity with minimized virulence have focused on merely shuffling codons in a virus while maintaining the amount of the shuffled codons that was present in the original virus (Coleman, J. R., et al., “Virus attenuation by genome-scale changes in codon pair bias”, Science, 320: 1784-1787 (2008)) or on replacing frequently used codon pairs with infrequently used but synonymous codon pairs (Conrad, S. J., et al., “Attenuation of Marek's disease virus by codon pair deoptimization of a core gene,” Virology, 516: 219-226 (2018)). No attempts have been made to use a systematic approach in optimizing codon replacement during viral deoptimization. Certainly no attempts have been made to find a global minimum related to unwanted virulence.

The present disclosure relates to a method for finding a global minimum CPB value to systematically optimize the deoptimization of viruses. The deoptimized virus will include a sequence corresponding to the global minimum CPB value. This sequence can be synthesized using methods known in the art for creating recombinant nucleic acid constructs. As a non-limiting example, recombinant nucleic acid constructs described herein may be synthesized using any known technique capable of synthesizing disclosed sequences. In such a non-limiting examples, techniques using direct integration may be used. In such a non-limiting example, techniques used by integrated DNA technology (IDT) may be used. IDT techniques use direct integration that is capable of ensuring repairs made according to the present disclosure meet applicable standards for synthesis. Techniques used by additional companies, such as Twist BioSciences or Eurofins, may also be used as long as set standards for synthesis are achieved. Many other synthetic processes are capable of being used according to the present disclosure. The deoptimized virus can then be placed into vaccine administrations and administered to subjects in need.

The present disclosure relates to a method involving preprocessing of a given sequence beginning at the first codon and stopping at the first stop codon based on the open reading frame (ORF) being preprocessed. This preprocessing will serve to remove most sequences containing mitochondrial sequences. Remaining mitochondrial sequences will be removed, as well as any sequences containing ambiguous nucleotides, which are used to identify unknown nucleotides (e.g. “N” may represent any possible nucleotide). Following preprocessing, CPS values can be calculated using codons, codon pairs, amino acids, and amino acid pairs using the formula described herein. CPS values can be assigned for genomes of specific species to be used in subsequent steps. As a non-limiting example, CPS values can be calculated for the genome of Gallus gallus. As a non-limiting example, CPS values can be calculated for protein coding genes of a species. As previously described, a lower CPS value indicates a rarer codon pairing in a genome. A lower CPB score, as previously described, indicates a lower total usage of a rarer codon pairing, as indicated by a low CPS.

In order to identify the best deoptimized sequence, a sequence corresponding to the lowest CPB value is desired. The lowest CPB value refers to the global minimum CPB value, which has not been calculated in previous methods. Finding the lowest (i.e. global minimum) CPB value is made difficult by the number of possible combinations of codons within a sequence. A sequence encoding for a protein of 200 amino acids in length (assuming 2 codons per amino acid) would yield 2²⁰⁰≈2×10⁶⁰ possible sequences. The magnitude of possible deoptimization requires improved methods of deoptimization.

The present disclosure relates to an improved method of deoptimization. Any amino acid that is encoded by only 1 codon can be used to split sequences into fragments. As a non-limiting example, GSLVLYPGMYDAGIYA can be split into GSLVLYPGM and MYDAGIYA where each resultant fragment is analysed individually. This greatly decreases the possible deoptimizations and the time needed to identify the deoptimized sequence with the global minimum CPB value. Once a sequence has been fragmented as described herein, each fragment is deoptimized to its global minimum and assigned CPB values to find the global minimum. Combinations are generated using a dynamic programming approach, moving left to right through the split sequences, building all possible combinations, and pruning those that are not useful. The splitting of the sequence greatly speeds up this process.

### IV. Methods of Repairing Deoptimized and Optimized Nucleic Acid Constructs

The present disclosure relates to methods of repairing nucleic acid constructs. Original attempts to deoptimize and optimize nucleic acid constructs according to the present disclosure presented drawbacks. As an example, sequences produced according to methods described herein occasionally included repeated sections of the sequences. Such repeat sections made the produced deoptimized or optimized sequence incapable of being synthesized or overly complex to synthesize if the repeat sections were undetected. The present disclosure provides several methods of fixing such errors associated with methods described herein. These methods of fixing errors further enhance and make operable other methods described herein. Methods of fixing errors may either provide for synthesis of a sequence, reduce the complexity of synthesis of a sequence, or both.

The present disclosure relates to methods of fixing errors in nucleic acid constructs by using reversion based repairs. All repair methods described herein use reversion based repairs, though such repairs may be used in different ways. Reversion based repairs may fix, as a non-limiting example, errors including repeats of sequences within one or more nucleic acid constructs. Fixing such errors may include removing or otherwise altering one or more errors to make a deoptimized or optimized nucleic acid construct capable of being synthesized, such as if a deoptimized or optimized construct is too complex to be synthesized. Overly complex constructs may result when such construct includes repeated sequences. As a non-limiting example, reversion based repairs may revert an original sequence on a per codon basis where individual codons are examined and reverted when required according to repair methods disclosed herein. Such original sequence may be trimmed prior to reversion based repairs. Such repairs result in a deoptimized or optimized sequence including fewer errors than the original sequence included prior to reversion based repair. As a non-limiting example, the deoptimized or optimized sequence may include 80%, 85%, 90%, 95%, or 100% fewer errors than the original sequence. A schematic representation of reversion based repairs is shown in FIG. 2.

Reversion based repairs may repair sequences in forward or reverse directions. As a non-limiting example, one pass of a reversion based repair may include two cycles. In one cycle of a repair pass, repeated k-mers are examined starting from largest to smallest and including 16 to 8. A “k-mer” refers to a subset of a given sequence where k-mers from 16 to 8 include subsequences of 16 units (i.e., 16-mer), 15 units (i.e., 15-mer), 14 units (i.e., 14-mer), and so on until 8 units (i.e., 8-mer). In such example, repeated k-mers of 16 units in length will first be repaired followed by repeated k-mers of 15 units in length, 14 units of length, and so on until 8 units in length. For each k-mer, the most common repeat k-mer is found. The most common repeat k-mer is then repaired using reversion to remove repeats. In a forward repair pass, repeated codons are reverted to codons from the original sequence (e.g., before optimization or deoptimization). In a reverse repair pass, reverted codons are reverted to codons from the optimized or deoptimized sequence. Repairs are made by reverting codons within each repeat. For the first repeat, the first codon is reverted. For the second repeat, the second codon is reverted and so forth until the nth repeat where the nth codon is reverted. If there are more repeats than codons, the codon to be reverted loops back to the first codon. As a non-limiting example, if there were an eighth repeat and only seven codons, the first codon would be reverted. After each repair, the next most common repeat k-mer is found. The most common repeat is recalculated at each step to account for overlaps of repeats. This technique continues until no unique repair can be identified. All repairs made to the sequence are tracked throughout each repair cycle to prevent the repair cycle from being stuck in a loop (e.g., where a repeat exists in both the original and deoptimized sequence). An additional cycle is then performed to complete a single pass. The additional repair cycle prevents the creation of additional repeats that may be created during the initial repair cycle. That is, reverting codons of continuously smaller k-mers may create new repeated k-mers requiring an additional repair cycle (e.g., the second repair cycle of a single pass).

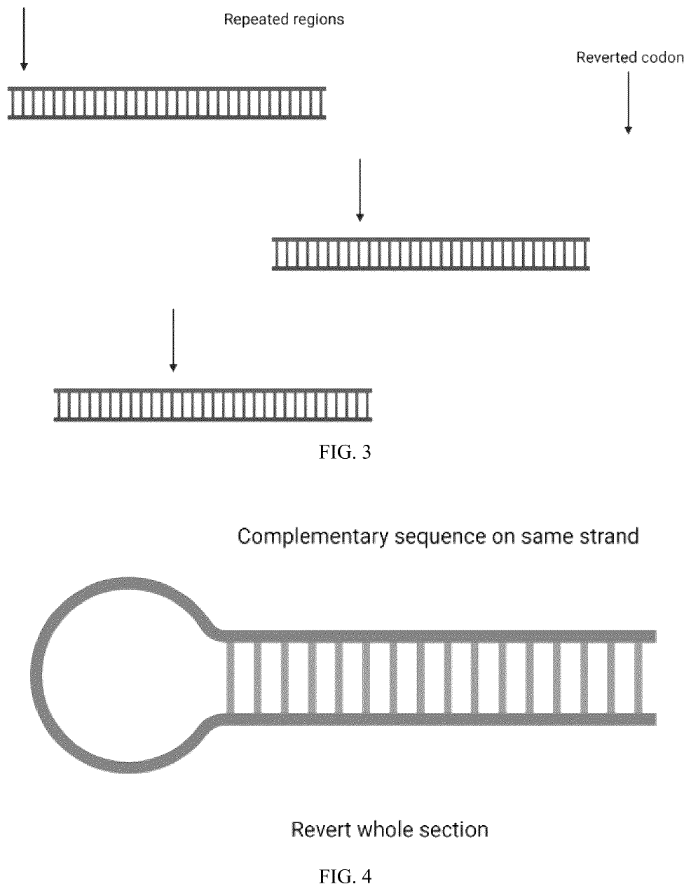

The present disclosure relates to methods of fixing errors in nucleic acid constructs including large repeat repairs. Large repeat repairs refer to examples of reversion based repairs applied to repeats of 8 or more amino acids where such repeats pose unique challenges. Such repairs may fix, as a non-limiting example, errors including repeats of sequences within one or more nucleic acid constructs. Large repeat repairs include removing or otherwise altering one or more errors to make a deoptimized or optimized nucleic acid construct capable of being synthesized. As a non-limiting example, large repeat repairs may transform repeat regions (i.e. errors) of an original sequence into reverted codons. Such original sequence may be trimmed prior to large repeat repairs. This repair method may occur in multiple cycles. Such cycles may operate by fixing repeated sequences ranging from about 8 to about 16 bases in each cycle, as discussed above. As a non-limiting example, large repeat repairs may require two or more cycles. In such an example, one cycle may include finding the most common repeat in a 16-base sequence, repairing it according to the present disclosure, finding the most common repeat in a 15-base sequence, repairing it, and so on until finding the most common repeat in an 8-base sequence, and repairing it to complete an initial cycle. Additional cycles may be completed since repairing codons from 16-base to 8-base sequences (from largest to smallest) may create additional repeats. Such repairs result in a deoptimized or optimized sequence including fewer errors than the original sequence included prior to large repeat repair. As a non-limiting example, the deoptimized or optimized sequence may include 80%, 85%, 90%, 95%, or 100% fewer errors than the original sequence. A schematic representation of reversion based repairs is shown in FIG. 3.

The present disclosure relates to methods of fixing errors in nucleic acid constructs by using hairpin repairs. Hairpin repairs refer to reversion based repairs applied to hairpin nucleic acid constructs that include forward and reverse sequences folded over onto each other. Hairpin repairs revert an entire complementarity region to its original sequence. This differs from other reversion based repair methods (e.g., large repeat repairs) disclosed herein that may be more precise by not reverting a whole region during repair. Other reversions may first revert a first codon of a first repeat and so on, as described above. Hairpin repairs may fix, as a non-limiting example, errors including repeats of sequences within one or more nucleic acid constructs. Fixing such errors may include removing or otherwise altering one or more errors to make a deoptimized or optimized nucleic acid construct capable of being synthesized. As a non-limiting example, hairpin repairs may operate on hairpin nucleic acid constructs, as shown in FIG. 4. In such an example, hairpin repairs are advantageous for fixing errors occurring in complementary nucleic acid sequences. Such errors may not be capable of being fixed by other repeat error methods described above. Hairpin repair methods may operate by reverting the whole section of a hairpin nucleic acid construct. This assists in fixing errors associated with complementary sequences included in the same strand of a nucleic acid construct. Such repairs result in a deoptimized or optimized sequence including fewer errors than the original sequence included prior to hairpin repair. As a non-limiting example, the deoptimized or optimized sequence may include 80%, 85%, 90%, 95%, or 100% fewer errors than the original sequence.

The present disclosure relates to methods of fixing errors in nucleic acid constructs by forward and backward repair methods. Such repairs may fix, as a non-limiting example, errors including repeats of sequences within one or more nucleic acid constructs. Fixing such errors may include removing or otherwise altering one or more errors to make a deoptimized or optimized nucleic acid construct capable of being synthesized. In such an example, forward and backward repairs may operate by using repair methods disclosed herein to repair an original sequence in a first direction. A repair method disclosed herein may then be run in the opposite direction of the first direction. Such process can be repeated several times in order to repair one or more sequences containing errors, as disclosed herein. Forward and backward repair methods may result repaired sequences with reduced synthetic complexity while also maintaining a desired deoptimization score, as described herein. Forward and backward repair methods may be particularly advantageous operating on original sequences including repeats and complexity issues. Repairing in a first direction and then in an opposite direction captures the best of both sequences by maximally maintaining the optimization or deoptimization of a sequence while eliminating all repeats. Forward and reverse repairs compensate for repeats that already exist in the original sequence. These repeats can prevent a sequence from being fully repaired by a single forward pass. Forward and reverse repairs result in a deoptimized or optimized sequence including fewer errors than the original sequence included prior to forward and backward repair. As a non-limiting example, the deoptimized or optimized sequence may include 80%, 85%, 90%, 95%, or 100% fewer errors than the original sequence.

The present disclosure relates to methods of maintaining deoptimized and optimized sequences. Repairs are needed when an optimized or deoptimized sequence has become too complex to be easily synthesized. Synthetic complexity may be caused by the introduction of repeat sequences in an overall sequence during optimization or deoptimization. Repairs may selectively revert codons (on a codon by codon basis) in repeat sequences to reduce synthetic complexity so that an optimized or deoptimized sequence can be synthesized. As such, levels of of optimization or deoptimization can be maintained during repair. Optimization and deoptimization can be shown by codon pair bias (CPB), as described herein. In some traditional examples, repair methods described herein may affect the CPB value of a sequence. In such examples, reversions occurring during sequence repair may make a deoptimized sequence less deoptimized or an optimized sequence less optimized, according to deoptimization and optimization methods of the present disclosure. In such examples, repair methods described herein can prevent effects on a deoptimized or optimized sequence's CPB value by “maintaining” said value. In a non-limiting example, forward and reverse based repairs described above may maintain a CPB value.

### V. Compositions of Deoptimized Infectious Laryngotracheitis Virus

The present disclosure relates to recombinant deoptimized viruses. Such viruses can be used in vaccines and other such treatments known in the art. Such viruses may include but are not limited to herpesviruses, alphaherpesviruses, alphaherpesvirus serotypes (e.g. serotypes 1-3), Marek's Virus, Infectious Laryngotracheitis Virus (ILTV), and the like. In a particular embodiment, ILTV may be deoptimized. Such viruses may be known to affect poultry including but not limited to chickens (e.g. broilers, pullets, layers, hens, etc.), turkeys, pheasants, and other such poultry known in the art.

Sequences for ILTV are known in the art. For example, the amino acid sequence of the envelope glycoprotein forming spikes at the surface of the virion envelope of ILTV (Gallid herpesvirus 1) strain 632 is:

MASLKMLICVCVAILIPSTLSQDSHGIAGIIDPRDTASMDVGKISFSEAI
GSGAPKEPQIRNRIFACSSPTGASVARLAQPRHCHRHADSTNMTEGIAVV
FKQNIAPYVFNVTLYYKHITTVTTWALFSRPQITNEYVTRVPIDYHEIVR
IDRSGECSSKATYHKNFMFFEAYDNDEAEKKLPLVPSLLRSTVSKAFHTT
NFTKRHQTLGYRTSTSVDCVVEYLQARSVYPYDYFGMATGDTVEISPFYT
KNTTGPRRHSVYRDYRFLEIANYQVRDLETGQIRPPKKRNFLTDEQFTIG
WDAMEEKESVCTLSKWIEVPEAVRVSYKNSYHFSLKDMTMTFSSGKQPFN
ISRLHLAECVPTIATEAIDGIFARKYSSTHVRSGDIEYYLGSGGFLIAFQ
KLMSHGLAEMYLEEAQRQNHLPRGRERRQAAGRRTASLQSGPQGDRITTH
SSATFAMLQFAYDKIQAHVNELIGNLLEAWCELQNRQLIVWHEMKKLNPN
SLMTSLFGQPVSARLLGDIVAVSKCIEIPIENIRMQDSMRMPGDPTMCYT
RPVLIFRYSSSPESQFSANSTENHNLDILGQLGEHNEILQGRNLIEPCMI
NHRRYFLLGENYLLYEDYTFVRQVNASEIEEVSIFINLNATILEDLDFVP
VEVYTREELRDTGTLNYDDVVRYQNIYNKRFRDIDTVIRGDRGDAIFRAI
ADFFGNTLGEVGKALGTVVMTAAAAVISTVSGIASFLSNPFAALGIGIAV
VVSIILGLLAFKYVMNLKSNPVQVLFPGAVPPAGTPPRPSRRYYKDEEEV
EEDSDEDDRILATRVLKGLELLHKDEQKARRQKARFSAFAKNMRNLFRRK
PRTKEDDYPLLEYPSWAEESEDE (SEQ ID NO:1, UniProt
accession number Q02409 which is incorporated
by reference in its entirety.)

### VI. Vaccine Compositions of Deoptimized Infectious Laryngotracheitis

The present disclosure relates to vaccine compositions containing deoptimized ILTV according to methods described herein. Vaccines can be administered to a subject in need to prevent, reduce, or treat a virus (e.g. ILTV). A subject includes but is not limited to poultry and other animals affected by ILTV. Such vaccines can be used to aid in the prevention and/or treatment of ILTV and any maladies and infections associated therewith. A vaccine according to the present disclosure can be used for prophylactic and/or for therapeutic treatment, and thus can interfere with the establishment and/or with the progression of an infection and/or its clinical symptoms of disease.

The present disclosure relates to deoptimized ILTV strains in vaccines. Such strains can be grown by any number of means currently practiced in the field. As a non-limiting example, in vitro cultures of primary chicken cells (e.g., chicken embryo fibroblast cells (CEFs) may be used. CEFs can be prepared by trypsinization of chicken embryos. CEFs also can be plated in monolayers and then infected with the deoptimized ILTV. This particular process can be readily scaled up to industrial-sized production. Therefore, a further aspect of the disclosure relates to a method for the preparation of the vaccine according to the disclosure comprising the steps of infecting host cells with deoptimized recombinant ILTV, harvesting the infected host cells, and then admixing the harvested infected host cells with a pharmaceutically acceptable carrier. Suitable methods for infection, culture and harvesting are well known in the art and are described and exemplified herein.

Typically, the infected host cells are harvested while still intact to obtain the deoptimized ILTV in its cell-associated form. These cells can be taken up in an appropriate carrier composition to provide stabilization for storage and freezing. The infected cells can be filled into glass ampoules, which are sealed, frozen and stored in liquid nitrogen. Accordingly, in certain embodiments of the present disclosure, the vaccines and/or immunogenic compositions of the present disclosure are stored frozen and accordingly, comprise a cryropreservative, such as dimethyl sulfoxide (DMSO), to preserve the frozen infected cells.

Alternatively, when the deoptimized ILTV is a recombinant HVT, it can be isolated from its host cell, for instance through sonication at the end of culturing, and then taken up into a stabilizer, and freeze-dried (lyophilized) for stable storage or otherwise reduced in liquid volume, for storage, and then reconstituted in a liquid diluent before or at the time of administration. Such reconstitution may be achieved using, for example, vaccine-grade water. In certain embodiments, a lyophilized portion of a multivalent vaccine can comprise one or more antigens and the diluent can comprise one or more other antigens.

In particular embodiments a vaccine of the present disclosure (or a portion thereof) can be in a freeze-dried form (e.g., as tablets and/or spheres that are produced by a method described in WO 2010/125084, hereby incorporated by reference in its entirety). In particular, reference is made to the examples, from page 15, line 28 to page 27, line 9 of WO 2010/125084, describing a method to produce such fast disintegrating tablets/spheres. Such freeze-dried forms can be readily dissolved in a diluent, to enable systemic administration of the vaccine.

Vaccines and immunogenic compositions can, but do not necessarily include, physiologically compatible buffers and saline and the like, as well as pharmaceutically acceptable adjuvants. Adjuvants can be useful for improving the immune response and/or increasing the stability of vaccine preparations. Adjuvants are typically described as non-specific stimulators of the immune system, but also can be useful for targeting specific arms of the immune system. One or more compounds which have this activity may be added to the vaccine. Therefore, particular vaccines of the present disclosure can further comprise an adjuvant. Examples of chemical compounds that can be used as adjuvants include, but are not limited to aluminum compounds (e.g., aluminum hydroxide), metabolizable and non-metabolizable oils, mineral oils including mannide oleate derivatives in mineral oil solution (e.g., MONTANIDE ISA 70 from Seppic SA, France), and light mineral oils such as DRAKEOL 6VR, block polymers, ISCOM's (immune stimulating complexes), vitamins and minerals (including but not limited to: vitamin E, vitamin A, selenium, and vitamin B12) and CARBOPOL®.

Other suitable adjuvants, which sometimes have been referred to as immune stimulants, include, but are not limited to: cytokines, growth factors, chemokines, supernatants from cell cultures of lymphocytes, monocytes, cells from lymphoid organs, cell preparations and/or extracts from plants, bacteria or parasites (Staphylococcus aureus or lipopolysaccharide preparations) or mitogens. Generally, an adjuvant is administered at the same time as an antigen of the present disclosure. However, adjuvants can also or alternatively be administered within a two-week period prior to the vaccination, and/or for a period of time after vaccination, i.e., so long as the antigen (e.g., a deoptimized ILTV of the present disclosure persists in the tissues).

The vaccines and/or immunogenic compositions of the present invention may be administered by any route such as in ovo, by water (embryo-derived also known as chicken embryo origin (CEO)), by spray or eye drop (cell culture derived also known as chicken embryo fibroblast (CEF)), by parenteral administration, including intramuscular injection, subcutaneous injection, intravenous injection, intradermal injection, by scarification, by oral administration, or by any combination thereof. Furthermore, the deoptimized ILTV of the present invention can be used and/or combined with additional antigens to improve and expand the immunogenicity provided, and/or antigens for other pathogens (e.g. Bursal Disease, Marek's Disease, Newcastle Disease, etc.) in order to provide immune protection against such other pathogens. These additional antigens can be either live or killed whole microorganisms, other recombinant vectors, cell homogenates, extracts, proteins, or any other such derivative, provided that they do not negatively interfere with the safety, stability, and efficacy of the vaccine according to the present disclosure. Vaccines can be administered at a variety of different times in the lifespan of a subject (e.g. poultry). As a non-limiting example, one or more vaccines can be administered at about 2 up to about 4 weeks of age. Vaccines may be administered in endemic areas. Further vaccinations may be provided at additional times in the life span of a subject. As a non-limiting example, pullets may be given additional vaccine doses at about 10 up to about 14 weeks. As a non-limiting example, additional vaccine doses may be used for poultry after said poultry has undergone molting.

Examples of other microorganisms that can be used as antigens together with the deoptimized ILTV of the present disclosure include: (i) viruses such as infectious bronchitis virus, adenovirus, egg drop syndrome virus, infectious bursal disease virus, chicken anemia virus, avian encephalomyelitis virus, fowl pox virus, turkey rhinotracheitis virus, duck plague virus (duck viral enteritis), pigeon pox virus, avian leucosis virus, avian pneumovirus, and reovirus, (ii) bacteria, such as Escherichia coli, Salmonella spec., Ornitobacterium rhinotracheale, Haemophilis paragallinarum, Pasteurella multocida, Erysipelothrix rhusiopathiae, Erysipelas spec., Mycoplasma spec., and Clostridium spec., (iii) parasites such as Eimeria spec., and (iv) fungi, such as Aspergillus spec.

In some examples, the deoptimized ILTV of the present disclosure can be combined with additional vaccines for additional pathogens. The combination vaccine can be made in a variety of ways including by combining the deoptimized ILTV of the present disclosure with preparations of virus, or bacteria, or fungi, or parasites, or host cells, or a mixture of any and/or all of these. In particular embodiments, the components for such a combination vaccine are conveniently produced separately and then combined and filled into the same vaccine container.

As described above, a vaccine according to the disclosure can be used advantageously to provide safe and effective immune protection in poultry to multiple diseases, by a single inoculation at very young age or in ovo. Alternatively, as would be apparent to anyone skilled in the art of poultry vaccines the combinations described above also could include vaccination schedules in which the deoptimized ILTV of the present disclosure and the additional antigen are not applied simultaneously (e.g. the deoptimized ILTV may be applied in ovo, and the additional pathogen vaccine could be applied at a subsequent time/date).

Accordingly, the vaccines of the present disclosure can be administered to the avian subject in combination with other poultry vaccines. The vaccine of the present disclosure can be administered with vaccines against infectious agents including, but not limited to, Marek's disease virus (MDV), Infectious Bursal Disease virus (IBDV), Newcastle Disease virus (NDV), Infectious Bronchitis virus (IBV), Infectious Laryngotracheitis virus (ILTV), Avian Encephalomyelitis virus (AEV), Chicken Anaemia virus (CAV), Fowlpox virus (FPV), Avian Influenza virus (AIV), Reovirus, Avian Leukosis virus (ALV), Avian Reticuloendotheliosis virus (REV), Avian Paramyxovirus (APV), Duck Hepatitis virus (DHV), Avian Adenovirus (AAV) and Hemorrhagic Enteritis virus (HEV). Vaccines against other infectious agents including, but not limited to, coccidiosis also are contemplated to be within the scope of the present invention.

### VII. Therapeutic and Preventative Compositions

The compositions below are to be understood as example compositions related to the present disclosure. Such are not intended to be limiting of the scope of the present disclosure.

The compositions described herein contain a deoptimized or attenuated sequence of a nucleic acid. The nucleic acid can be that of a virus including but not limited to ILTV. Deoptimized nucleic acids can be administered in vaccines as recombinant vectors. Alternatively, proteins, fusion proteins, and any fragments thereof encoded by deoptimized nucleic acid sequences can be administered by vaccine. The compositions described herein can be administered to a host, either alone or in combination with a pharmaceutically acceptable excipient, in an amount sufficient to prevent or treat a disease, wherein the disease may be caused by a virus or other known pathogen. The response can comprise, without limitation, specific immune response, non-specific immune response, both specific and non-specific response, innate response, primary immune response, adaptive immunity, secondary immune response, memory immune response, immune cell activation, immune cell proliferation, immune cell differentiation, and cytokine expression.

### A. Methods of Use

a. Dosing and Timing Regimens

Accordingly, the vaccines of the present disclosure can be administered to an avian subject in a single dose or in multiple doses. For example, a vaccine of the present disclosure may be applied at the day of hatch and/or in ovo at day 16-18 (Embryonation Day) ED. When multiple doses are administered, they may be given either at the same time or sequentially, in a manner and time compatible with the formulation of the vaccine, and in such an amount as will be immunologically effective. Therefore, a vaccine of the present disclosure may effectively serve as a priming vaccination, which later can be followed and amplified by a booster vaccination of the identical vaccine, or with a different vaccine preparation (e.g. a classical inactivated, adjuvanted whole-virus vaccine). Administration can include injection, infusion, other methods disclosed herein, and other methods known in the art. Administration includes but is not limited to intravenous, intramuscular, subcutaneous, and the like.

As described above, an effective amount of the compositions described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the compositions. Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The disclosure is not limited to dosing intervals that are spaced equally in time, but encompass doses at non-equal intervals, such as a priming schedule consisting of administration at 1 day, 4 days, 7 days, and 25 days, just to provide a non-limiting example. In such aspects, various compositions can be administered using different dosing and spacing regiments. In such aspects, a first composition may be administered in one or more doses spaced at certain time intervals while a second composition may be administered in a different number of doses spaced at different time intervals. In such an aspect, a first composition and second composition may differ in makeup. Dosing and spacing regiments may also be influenced by endemic outbreaks of a pathogen. As a non-limiting example, dosing and spacing regiments may be influenced by an outbreak of ILTV in a flock of poultry.

The volume per dose of a vaccine of the present disclosure can be optimized according to the intended route of application. In ovo inoculation is commonly applied with a volume between about 0.05 ml/egg and about 0.5 ml/egg, and parenteral injection is commonly done with a volume between about 0.1 ml/avian and about 1 ml/avian. In any case, optimization of the vaccine dose volume is well within the capabilities of the skilled artisan, as described below.

The therapeutically effective dosage of a deoptimized pathogen can be provided as repeated doses within a prolonged prophylaxis or treatment regimen that will yield clinically significant results to alleviate one or more symptoms or detectable conditions associated with a targeted disease or condition as set forth herein. Determination of effective dosages are typically based on animal model studies followed up by human clinical trials and is guided by administration protocols that significantly reduce the occurrence or severity of targeted disease symptoms or conditions in the subject. Various considerations are described, e.g., in Gilman et al., eds., Goodman and Gilman: The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference. Suitable models in this regard include, for example, murine, rat, porcine, feline, non-human primate, and other accepted animal model subjects known in the art.

Immunologically effective dosages can also be determined using in vitro models (for example, immunologic and histopathologic assays). Using such models, only ordinary calculations and adjustments are used to determine an appropriate concentration and dose to administer a therapeutically effective amount of the deoptimized pathogen (for example, amounts that are effective to elicit a desired immune response or alleviate one or more symptoms of a targeted disease). In some examples, amounts administered are those amounts adequate to achieve tissue concentrations at the site of action which have been found to achieve the desired effect in vitro. In alternative examples, an effective amount or effective dose of the deoptimized pathogens can decrease or enhance one or more selected biological activities correlated with a disease or condition.

The actual dosage of the deoptimized pathogen can vary according to factors such as the disease indication and particular status of the subject (for example, the subject's age, weight, fitness, extent of symptoms, susceptibility factors, and the like), time and route of administration, the type of pathogen against which vaccination is sought, other drugs or treatments being administered concurrently, as well as the specific pharmacology of the deoptimized pathogens for eliciting the desired activity or biological response in the subject. Dosage regimens can be adjusted to provide an optimum prophylactic or therapeutic response. A therapeutically effective amount is also one in which any toxic or detrimental side effects of a deoptimized pathogen are outweighed in clinical terms by therapeutically beneficial effects.

A dosing schedule of, for example, once/week, twice/week, three times/week, four times/week, five times/week, six times/week, seven times/week, once every two weeks, once every three weeks, once every four weeks, once every five weeks, and the like, is available for the disclosure. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

An effective amount of a therapeutic or preventative agent is one that will decrease, ameliorate, or prevent the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

Formulations of therapeutic or preventative agents may be prepared for storage by mixing with physiologically acceptable carriers, excipients, or stabilizers in the form of, e.g., lyophilized powders, slurries, aqueous solutions or suspensions.

b. Therapeutic Strategies

Methods of inducing or enhancing an immune response in a subject are provided. Typically, the methods include administering a subject an effective amount of immunomodulatory agent, or cells primed ex vivo with the immunomodulatory agent. The immune response can be, for example, a primary immune response to an antigen or an increase effector cell function such as increasing antigen-specific proliferation of T cells, enhancing cytokine production by T cells, stimulating differentiation, or a combination thereof. In some embodiments, the agent can increase the development of naïve T cells into Th1, Th17, Th22, or other cells that secrete, or cause other cells to secrete, inflammatory molecules, including, but not limited to, IL-10, TNF-α, TGF-beta, IFN-7, IL-17, IL-6, IL-23, IL-22, IL-21, and MMPs. In some embodiments, the agent can reduce or inhibit the activity of Tregs, reduce the production of cytokines such as IL-10 from Tregs, reduce the differentiation of Tregs, reduce the number of Tregs, reduce the ratio of Tregs within an immune cell population, or reduce the survival of Tregs. The immunomodulatory agent can be administered to a subject in need thereof in an effective amount to overcome T cell exhaustion and/or T cell anergy. Overcoming T cell exhaustion or T cell anergy can be determined by measuring T cell function using known techniques.

The methods can be used in vivo or ex vivo as immune response-stimulating therapeutic applications. Thus in some embodiments, the agent, or nucleic acid encoding the agent, is administered directly to the subject. In some embodiments, the agent or nucleic acid encoding the agent, is contacted with cells (e.g., immune cells) ex vivo, and the treat cells are administered to the subject (e.g. adoptive transfer). In general, the disclosed immunomodulatory agents can be used for treating a subject having or being predisposed to any disease or disorder to which the subject's immune system mounts an immune response. The agents can enable a more robust immune response to be possible. The disclosed compositions are useful to stimulate or enhance immune responses involving T cells.

### B. Methods of Manufacture

a. Methods for Producing Proteins

The disclosed proteins, polypeptides, fragments, variants and fusions thereof can be manufactured using conventional techniques that are known in the art. Isolated fusion proteins can be obtained by, for example, chemical synthesis or by recombinant production in a host cell. To recombinantly produce a protein, polypeptide, fragment, variant or fusion thereof, a nucleic acid containing a nucleotide sequence encoding the protein, polypeptide, fragment, variant or fusion thereof can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the protein, polypeptide, fragment, variant or fusion thereof. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express fusion proteins. Viral based expression systems are well known in the art and include, but are not limited to, baculoviral, SV40, retroviral, or vaccinia based viral vectors.

Mammalian cell lines that stably express proteins, polypeptides, fragments, variants or fusions thereof, can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of proteins, polypeptides, fragments, variants or fusions thereof, in, for example, Chinese hamster ovary (CHO) cells, COS-1 cells, human embryonic kidney 293 cells, NIH3T3 cells, BHK21 cells, MDCK cells, and human vascular endothelial cells (HUVEC). Additional suitable expression systems include the GS Gene Expression System™ available through Lonza Group Ltd.

Following introduction of an expression vector by electroporation, lipofection, calcium phosphate, or calcium chloride co-precipitation, DEAE dextran, or other suitable transfection method, stable cell lines can be selected (e.g., by metabolic selection, or antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a protein, polypeptide, fragment, variant or fusion thereof, can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Proteins, polypeptides, fragments, variants or fusions thereof, can be isolated using, for example, chromatographic methods such as affinity chromatography, ion exchange chromatography, hydrophobic interaction chromatography, DEAE ion exchange, gel filtration, and hydroxylapatite chromatography. In some embodiments, Proteins, polypeptides, fragments, variants or fusions thereof can be engineered to contain an additional domain containing amino acid sequence that allows the polypeptides to be captured onto an affinity matrix. For example, an Fc-fusion polypeptide in a cell culture supernatant or a cytoplasmic extract can be isolated using a protein A column. In addition, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify polypeptides. Fusion proteins can additionally be engineered to contain a secretory signal (if there is not a secretory signal already present) that causes the proteins, polypeptides, fragments, variants or fusions thereof to be secreted by the cells in which it is produced. The secreted proteins, polypeptides, fragments, variants or fusions thereof can then conveniently be isolated from the cell media.

b. Methods for Producing Isolated Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a variant polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also be obtained by mutagenesis. Protein-encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992.

Although several aspects have been disclosed in the foregoing specification, it is understood by those skilled in the art that many modifications and other aspects will come to mind to which this disclosure pertains, having the benefit of the teaching presented in the foregoing description and associated drawings. It is thus understood that the disclosure is not limited to the specific aspects disclosed hereinabove, and that many modifications and other aspects are intended to be included within the scope of any claims that can recite the disclosed subject matter.

It should be emphasized that the above-described aspects are merely possible examples of implementations, merely set forth for a clear understanding of the principles of the present disclosure. Any process descriptions or blocks in flow diagrams should be understood as representing modules, segments, or portions of code which comprise one or more executable instructions for implementing specific logical functions or steps in the process, and alternate implementations are included in which functions may not be included or executed at all, can be executed out of order from that shown or discussed, including substantially concurrently or in reverse order, depending on the functionality involved, as would be understood by those reasonably skilled in the art of the present disclosure. Many variations and modifications can be made to the above-described aspect(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

### EXAMPLES

### Example 1: Codon Pair Bias for Deoptimization and Optimization

Codon pair bias was based on the Gallus gallus genome and filtered for protein coding at (“Genes: Gallus gallus”, National Center for Biotechnology Information). The frequency of all codons, codon pairs, amino acids, and amino acid pairs were calculated. Lower codon pair scores indicated rarer codon pairing or more deoptimization. Dynamic programming was used to generate approximately eight amino acid sequences where an exhaustive search across the gene was conducted (Huang, Y., et al., “Codon pair optimization (CPO): a software tool for synthetic gene design based on codon pair bias to improve the expression of recombinant proteins in Pichia pastoris”, Microbial Cell Factories, 20: 209 (2021)). Similarly, codon pair optimization for increased expression of protein can be performed in which the higher the codon pair score indicates frequent codon pairing or more optimization.

### Example 2: Codon Pair Deoptimization or Optimization and Repair

Codon pair deoptimization or optimization was performed according to the following methods. Repairs of sequences were also performed according to the below methods when repairs were performed.

Sequences were provided along with parameters including the host profile to deoptimize or optimize for depending on the desired process, whether to deoptimize or optimize, and the target deoptimization or optimization percentage.

A first stage included a deoptimization or optimization method. DNA sequences were trimmed from the first start codon to the first stop codon. The sequences were sliced up at AAs that could only be represented by a single codon, and thus could not be deoptimized or optimized. This allowed multiple shorter sequences to be formed that could be deoptimized or optimized in parallel.

For very short fragments (such as fragments of 13 AAs or shorter), all possible combinations were exhaustively enumerated and scored, with the highest scoring (for optimization) or lowest scoring (for deoptimization) fragment selected. All other fragments underwent dynamic programming based deoptimization or optimization algorithms. Each sequence fragment was sliced into smaller fragments (subfragments) of 6 AAs or less.

For each subfragment, the first and last (outer) codons were iteratively fixed for all their possible values. The remaining middle codons were exhaustively enumerated, and the entire subfragment was scored. The highest scoring (for optimization) or lowest scoring (for deoptimization) subfragment for each fixed outer codon pair was retained.

This list of solutions represented the most deoptimized or optimized codon choices possible for a given subfragment, irrespective of the codons present in the surrounding sequence. This solution list was memoized using a hash table, meaning for a given subfragment no further recalculations of deoptimization or optimization were required in the future.

These subfragments were then recombined by taking the cartesian product of each pair of subfragments. These new solutions were grouped by their outer codons, and only the best solution for each group was retained and added to the hash table.

This process was repeated for each subfragment until the entire fragment had been reconstructed. The best solution for the fragment was calculated by taking the highest score (for optimization) or lowest score (for deoptimization) since a fragment was guaranteed to have no neighboring codon that could affect the best solution for that fragment.

The sequence was then rebuilt from the various deoptimized or optimized fragments. This final sequence was scored and returned according to the method.

A second, optional stage reverted codons for partial deoptimization or optimization. This was a conditional step that only applied if there was a deoptimization or optimization percentage below 100%. The difference in the percentage of codons (e.g., if a target percentage for deoptimization or optimization was 80%, then 20% of codons were selected) were selected in a deterministic pseudorandom fashion. The selected codons were reverted to their original sequence identity. No regard was made of codons that were same in the original sequence and the deoptimized or optimized sequence. As an example, if the deoptimization percentage was 50%, then half of all codons would be reverted to their original. These sequences were scored and then returned by the algorithm.

A third stage included repair of sequences for synthesis. This stage checked integrated DNA technology (IDT) synthesis application programming interface (API) for any identified issues with sequences. If a hairpin construct was identified, the entire hairpin was reverted to the original sequence. Repairs of sequences could occur by a forward repair pass followed by a reverse repair pass followed by a forward repair pass.

In a forward repair pass, reverted codons are reverted to codons from the original trimmed sequence. In a reverse repair pass, reverted codons are reverted to codons from the deoptimized or optimized sequence from the second stage.

A repair pass involved two cycles including repeated k-mers (i.e. subsequences of length) of 16 to 8 inclusive (starting from largest to smallest) units. For each k-mer the most common repeat was found. For each set of repeated k-mers, a repair was attempted. After each repair, the next most common repeat was found (this was recalculated at each step as repeats may overlap). This process was repeated until no unique repair could be found. All repairs made to the sequence were tracked throughout a repair cycle to prevent indefinitely looping the process (e.g., where a repeat exists in both the original and deoptimized sequence).

Repairs were made by reverting codons within each repeat. For a first repeat, the first codon was reverted. For a second repeat, the second codon was reverted. This continued as needed where the n^{th} codon would be reverted for the n^{th} repeat. If there were more repeats than codons in a repeat, the reversion looped back to the first codon. As an example, if there were 4 repeats of 3 codons, the 4th repeat would result in the reversion of the 1st codon.

Once there were no more unique repairs available, the process proceeded to the n−1 k-mer length. The final repaired sequence was re-scored by the IDT API and outputted as the final sequence for synthesis.

### Example 3: Codon Pair Deoptimization of ILTV Polymerase Gene

Using sequences obtained from NCBI reference sequence number NC_006623.1, located between nucleotides 47271-50582 the polymerase gene (UL30) consists of 3312 nucleotides. Targeting the polymerase gene for codon pair deoptimization would slow the replication of the virus by slowing the synthesis of DNA.

The sequence provided below is known in the art as the wild type polymerase gene (UL30). The codon pair score of the following sequence was calculated to be −0.0612.

ATGGACACTTTTTCCTCCGCCGGTACTGGAGCTGCTCGTGTAGCCCCGAT
AATTGGCCTCGGGCCGCACTCTGGCGCGTCTTATTATACTTCTGTGAGAG
AATTCCCGTATGTATGTCCAACGTGCATTAATGGGGGCGGAAGGATAGGA
ACGCAGATTGGAAGGGCTACAAGCAAACCAACCTTTTACCACAACGAGCG
ACAATTAGACATCTTGACAAGCACACATGGAGCGTGGCCCGTCAGGATGA
AATATTGGAATGCTGGTCCTGGAATGACTCACCACAACAAGGGAGATGTA
AAGTTTTATGAATTTCACGTTTATGACCTTTTTGAAAACACTGAGATCGC
TCAGTCATGTGTACGATGGATGCATTCCAGATTTCTTGATACACTGAAAC
CGACCGGTACTGTAATAACCTTAGTTGGAAATTCTGCATGCGGAAAGCGC
GTCGCCGTTCATGTGTATGGATCTTTGCCATACTTTTTTGTTAAAAAGCG
AGAAATTGACCAAGCCGCGAAAGTCACGAACTCGGAAGAGTTGGCGCATG
CTCTAGCATTAGCGACGCGTAAGACATCTTTAAAAAATTCACCTTTTCTA
GCTGTTTCAGCGGAAAGTTTTTGCATTGATGTTGTTCGGCGCAGAGACAT
TTATTATTCTGAATCTGAGGAGGAGGACTTCTACCGTGTAAAAGTGTGTA
ATGGCAAAGTGATGAAATTTATATGTGATAATTTTTTCCCGAGCGTACCT
AAATATGAAAGCAATGTTGACGCTATTACCAGGTTCATCCTCGACAATAA
TCTGACTTCTTTTGGGTGGTATCGCTTTAGAGCTCAAGGTGGCGCACTCC
AAATTCGAGACCCAGGACAACATGCTACATCAGCTGATGTCGAAATTAAC
TGCACAGCAAGTAATTTGGAACTCGGAAACAATGTCTCGTGGCCAGACTA
CAAATTACTGTGCTTTGATATAGAATGTAAAGCCGGAGGGGCAAATGAGT
TTGCATTTCCTTCTGCTGAGAAGGTAGAAGATCTTGTTATCCAAATTTCG
GCAGTTACATATTCTTTGTTAACTAAGGAAAAAGAGCAGGAAATCATTTT
TTCCCTAGGGACCTGTGAACTTTCCGAAGATTGTAGTGATGGGATAACTG
TATGCAAATGTGGCTCTGAGTTCGAGCTTCTTCTGTGCTTTATGACTTTT
TTCAAGCAATATTCACCCGAATTTGTGAGCGGATACAATATTCTAGGGTT
CGATTGGGCCTATCTTTCTAACAAGCTGAGCAAGGTGTATGGCATGCGTC
TCGATGGATATGGGAAAGCAAACTCGTGGGGGACATTCAAACTTCAAGAT
CCGTCTGCGCGCGGACTTGGAAGATTCAAAAAAGTCAAAATCAATGGCGT
CGTCAACATTGATATGTTTACCATTAGTTATGAAAAGCTAAAGTTACCAT
CTTATAAGTTAAATGCAGTAGCAGAGTGCGTTCTTGGAGAACAAAAAATT
GATCTGGCTTACAAAGATATCCCAGTCATGTTTGCTGCAGGCCCTAAAGA
AAGGGGGAAAATAGGGGAGTATTGTTTGCAGGATTCTAGACTGTCAGGGA
GTTTGTTTTTCAAATTCTCCCCTCATTTAGAAATGTCCGCTGTCGCTAAA
TTGGCCTGTATACCCTTGACAAGAGCAATAGGCGATGGACAGCAGCTGCG
CGTTTATACATGCTTGCTCCAACGCTCTACGGCGTCCGGGTTTGTGTTGC
CGGACAAAAAGAGTGCGTTTTCCTTCGGTTCTACTCTTGCTTCAGACGCG
TCTGATGCACCTGCTACAAGAAATGTAGGCTACCAGGGAGCCAAGGTACT
AGATCCAGAAATAGGATTCCATGTAACTCCTGTGGTTGTATTCGATTTTG
CCAGTTTGTATCCGAGTATTATTCAGAGTAACAATCTATGTTACAGTACG
TTAACGCACGACCCCGCCGCACTGGCAGGTCTACAGCCAGAAAAAGATTT
CTTCCAGATTGATGTTCAGGGTCGGAAATTTTATTTTGTCAGGGAGCATA
TTCGCAAAAGTTTGCTGTCAGACTTGCTAGGTGATTGGTTATCTATGAGG
AAAGCCGTGCGCGCAAAAATCAAGACAGCCGAGACTGAAGAAGAGCGAAT
CTTATTAGACAAACAGCAAGCTGCCTTAAGTTGTCTGGAACTCTGCTATG
GGTTTACTGGGGTGATGCATGGAATGCTTCCTTGTTTAGAAGTTGCTTCA
ACAGTTACAGCTATAGGAAGGGACATGCTTTTGCGTACAAAGGCGCACAT
CGAGAAGGAGTGGAGAAGTGGAAATCAATTTGCCGAAAAATTTTTGCCCG
GGTCCGAACGTATTCAGCTAAACCAATACTCTGTCCGGGTCATTAATGGA
GAAACTGATTCTGCATATTGTGAAGTTTACCGGAGTTTACATTTAAACTT
TAACTCAAGTTGGTGGCTGCATGGCCGAATAGATTTAAACAAGCGCTTTT
TAGAGGTCCGGGTAAAACTTGAGTGGAAAAGATTTTCGTGCGCCCTGTTA
CTCATTGGTAAGGAAAAATACATTGGGGTGATGCATGGAGGTAAAATGTT
GATGAAGGGCGTAGATTTAGTCAGGAAAACAAACTGTAAATTTGTCAATA
CCACAGCCTCCCGATTAGTGAATCTACTTTTCGAAGACGACGAAATAGCG
ATGGCTGCAGATACAGCAAGAGCCGGATTTGAGGATTTTGACTGTCTTCC
GAGCGGACTTACTAAGCTTGGCCGGTTGATTGCAGAGGCAAGGCTAGCTA
TTACCGGCAACGGACTAAACATTAGGGACTTCATAATGACCGCGGAATTA
AGTCGTGCGGTGGATAACTATGCCAGTTTGAAATTGCCTCATCTCACGGT
TTATCAAAAGAAAGCTGCTCCGCGCGAAGAATTACCTCCAATTAAAGAAA
GAATCGAATATGTCATCATAGAACCGGGACAATCTGTCCCGAATGATCCA
GCAACTGAGTCTTTTCCTAACTAAAAGAAACTTCTTAAATTTCTTAGCCT
GGCAGAAGATCCAGAATGGGTCGTATCAAATGGTCTAAAATTGAATGTAG
AATACTACTTTGATTCGCTTATTAGAACATTAAGTGTAACATTCAACGCA
ATTTTCGGTGATGCAAAAACAGCAGAGGATGTTTTAAGAAGCTTTATTCC
AGAGAAAATACAGTTTTCTGAGAAAGTTGCAGAGGCTCTTGCACGAAACA
CCGCGACATTTGTATCCATCCAGAAGTAATCGGAGGGCTGTAAGTTGCAA
ATGTTGAATAAA (SEQ ID NO: 2, residues 47271-50582
of NCBI reference sequence number NC_006623.1
which is incorporated by reference in its
entirety.)

The sequence provided below is a non-limiting example of a fully deoptimized version of SEQ ID NO: 2. Deoptimization was performed on the ORF according to methods of the present disclosure. The codon pair score of the following sequence was calculated to be −0.7514.

ATGGATACGTTTTCCAGTGCGGGTACGGGTGCTGCTAGGGTCGCACCGAT
AATCGGCCTCGGGCCGCATTCGGGTGCGTCGTATTATACGTCTGTGAGAG
AATTTCCGTACGTATGTCCAACATGCATTAACGGTGGGGGACGTATAGGA
ACGCAGATCGGTAGGGCGACTTCGAAACCGACATTCTACCATAACGAACG
CCAACTCGATATACTTACATCGACACATGGCGCATGGCCCGTCCGTATGA
AATATTGGAACGCTGGTCCGGGTATGACGCATCACAACAAGGGAGACGTA
AAGTTTTACGAATTTCACGTATACGACCTATTCGAAAATACTGAAATAGC
GCAATCGTGCGTACGTTGGATGCATTCGCGATTTCTCGATACACTAAAAC
CGACCGGTACTGTAATTACGCTAGTTGGAAATTCGGCATGCGGTAAGCGC
GTCGCGGTACACGTGTACGGATCTCTACCGTACTTTTTTGTAAAAAAACG
CGAAATCGATCAAGCGGCTAAGGTCACGAATTCGGAAGAGCTAGCGCATG
CGCTAGCATTAGCGACGCGTAAGACATCGTTAAAAAATTCGCCGTTTCTA
GCTGTATCAGCGGAATCGTTTTGCATTGACGTTGTACGTCGCAGAGATAT
ATATTATTCTGAATCTGAAGAGGAGGACTTTTATCGTGTAAAAGTGTGTA
ACGGTAAGGTGATGAAATTCATATGCGATAACTTTTTCCCGAGCGTACCG
AAATACGAATCGAACGTTGACGCTATTACGCGATTCATCCTCGATAATAA
CCTAACGTCTTTCGGTTGGTATCGGTTTAGAGCTCAAGGGGGCGCGCTAC
AAATTCGAGATCCAGGACAACACGCGACTAGCGCGGACGTCGAAATAAAT
TGTACGGCTTCGAATTTGGAACTCGGAAATAATGTATCGTGGCCGGATTA
CAAGTTACTATGTTTCGATATCGAATGTAAGGCGGGTGGGGCGAACGAGT
TCGCATTTCCGTCTGCTGAAAAGGTAGAAGACCTCGTAATCCAAATATCG
GCGGTAACATATTCGTTGTTAACGAAAGAGAAAGAGCAAGAAATCATCTT
TTCGCTAGGTACGTGCGAACTATCGGAAGATTGTTCGGATGGTATAACTG
TATGCAAGTGCGGCTCTGAATTCGAACTTCTACTGTGCTTTATGACGTTT
TTTAAGCAATATAGTCCGGAATTCGTATCCGGATATAACATACTAGGGTT
CGATTGGGCGTATCTTTCGAATAAGCTATCGAAGGTATATGGTATGCGAC
TCGACGGATACGGGAAAGCGAACTCGTGGGGTACGTTCAAACTCCAAGAT
CCGAGTGCGCGTGGACTTGGTCGGTTTAAAAAGGTTAAGATCAATGGCGT
AGTCAATATCGATATGTTTACGATTAGTTACGAAAAACTTAAGTTACCGT
CTTATAAATTAAATGCAGTAGCAGAATGCGTTCTAGGAGAACAAAAAATT
GATCTAGCGTATAAAGACATACCGGTAATGTTCGCAGCGGGCCCGAAAGA
AAGGGGTAAAATAGGGGAATATTGTCTACAAGATTCTAGACTGTCGGGTA
GTCTATTCTTTAAGTTTAGTCCTCATTTAGAAATGTCCGCTGTAGCTAAA
CTAGCGTGTATACCGTTAACACGAGCAATAGGCGACGGTCAGCAACTGCG
CGTATATACGTGTCTACTCCAACGATCGACCGCGTCCGGATTCGTACTAC
CGGATAAAAAGTCGGCGTTTTCGTTCGGTTCGACCCTTGCGTCCGACGCG
TCTGATGCACCTGCTACGAGAAACGTAGGGTATCAAGGTGCCAAAGTACT
AGATCCGGAAATCGGATTCCACGTAACGCCCGTAGTCGTATTCGATTTCG
CCTCGCTATATCCGTCGATTATACAAAGTAACAATCTATGTTATAGTACC
CTAACGCACGACCCGGCCGCACTGGCGGGTCTACAACCAGAAAAAGATTT
CTTCCAAATTGATGTACAGGGGCGTAAATTTTACTTCGTAAGGGAACATA
TACGCAAAAGTTTGCTGTCGGACCTACTAGGCGATTGGTTATCGATGAGG
AAAGCCGTGCGCGCGAAAATTAAGACAGCCGAGACGGAAGAAGAGCGCAT
ACTATTAGATAAGCAGCAAGCTGCCCTAAGTTGTCTGGAACTCTGTTATG
GTTTTACTGGGGTAATGCACGGAATGCTTCCGTGTCTCGAAGTCGCTTCG
ACCGTAACCGCGATAGGTCGCGACATGCTATTGCGTACAAAAGCGCATAT
CGAAAAGGAGTGGCGATCGGGTAATCAATTTGCCGAAAAATTCCTACCGG
GTTCCGAACGTATACAATTGAACCAATATTCTGTCCGCGTAATTAATGGC
GAAACTGATAGTGCGTATTGCGAAGTATACCGAAGTCTACATTTAAACTT
TAATTCAAGTTGGTGGTTACACGGGCGAATAGACTTAAACAAGCGGTTTT
TAGAGGTCCGGGTAAAACTCGAATGGAAAAGATTTTCGTGTGCCCTATTA
CTAATTGGTAAGGAAAAATATATAGGCGTGATGCATGGAGGTAAGATGCT
AATGAAAGGCGTAGATCTCGTCCGTAAAACGAATTGTAAGTTCGTCAATA
CCACCGCTTCCCGACTCGTAAACCTACTTTTCGAAGACGACGAGATAGCG
ATGGCTGCCGATACGGCAAGAGCGGGTTTTGAAGATTTCGACTGTCTTCC
GAGCGGTCTAACTAAGCTTGGCCGCCTAATCGCAGAAGCACGTCTAGCGA
TTACGGGTAACGGACTAAACATAAGGGATTTTATAATGACCGCGGAATTA
TCGCGTGCGGTCGACAATTATGCGAGTCTAAAGTTGCCGCATCTAACGGT
TTACCAAAAAAAAGCGGCGCCACGCGAAGAGTTACCGCCAATAAAGGAAA
GAATCGAATACGTAATCATCGAACCGGGACAATCGGTCCCGAACGATCCA
GCAACGGAGTCGTTTCCGAAT (SEQ ID NO: 3, deoptimized
version of SEQ ID NO:2 ORF obtained from NCBI
reference sequence number NC_006623.1 which is
incorporated by reference in its entirety.)

### Example 4: Codon Pair Deoptimization of ILTV Glycoprotein J Gene

Using sequences obtained from NCBI reference sequence number NC_006623.1, located between nucleotides 129739-135004 the glycoprotein J gene (US5) consists of 5266 nucleotides. Glycoprotein J is an envelope protein that is abundant on the virus surface. Deletion of gJ causes minor effects on direct cell to cell spread, while the virus titers were significantly reduced and in vivo was significantly attenuated compared to the wild type virus (Fuchs, W., et al., “In Vitro and In Vivo Relevance of Infectious Laryngotracheitis Virus gJ Proteins That Are Expressed from Spliced and Nonspliced mRNAs”, Journal of Virology, 79: 705-716 (2005)).

The below sequence shows the wild type sequence of the glycoprotein J gene (US5). The codon pair score for this sequence was calculated to be −0.1562.

ATGGGGACAATGTTAGTGTTGCGCCTTTTCCTACTTGCAGTAGCGGACGC
GGCGTTGCCGACCGGCAGATTCTGCCGAGTTTGGAAGGTGCCTCCGGGAG
GAACCATCCAAGAGAACCTGGCGGTGCTCGCGGAATCGCCGGTCACGGGA
CACGCGACATATCCGCCGCCTGAAGGCGCCGTCAGCTTTCAGATTTTTGC
GGACACCCCTACTTTGCGCATTCGCTACGGCGCTACGGAGGACGAACTTG
CACTGGAGCGCGGGACGTCCGCCTCAGACGCGGACAACGTGACATTTTCG
CTGTCATATCGCCCGCGCCCAGAAATTCACGGAGCATACTTCACCATAGG
GGTATTCGCTACTGGCCAGAGCACGGAAAGCAGCTATTCGGTCATCAGTC
GGGTCTTAGTTAACGCCTCTCTGGAACGGTCCGTGCGCCTGGAAACGCCG
TGCGATGAAAATTTTTTGCAGAACGAGCCTACATGGGGCTCGAAGCGTTG
GTTAGGCCCCCCGTCGCCTTATGTGCGAGATAACGATGTCGCCGTGTTGA
CAAAAGCGCAGTACATTGGGGAGTGCTACTCCAACTCGGCGGCCCAGACG
GGGCTCACGTCTCTCAACATGACCTTTTTCTATTCGCCTAAAAGAATAGT
AAACGTCACGTGGACAACCGGCGGCCCCTCCCCCTCGCGCATAACGGTAT
ACTCGTCGCGGGAGAACGGGCAGCCCGTGTTGAGGAACGTTTCTGACGGG
TTCTTGGTTAAGTACACTCCCGACATTGACGGCCGGGCCATGATAAACGT
TATTGCCAATTATTCGCCGGCGGACTCCGGCAGCGTCCTCGCGTTTACGG
CCTTTAGGGAAGGAAAACTCCCATCCGCGATTCAACTGCACCGGATAGAT
ATGTCCGGGACTGAGCCGCCGGGGACTGAAACGACCTTCGACTGTCAAAA
AATGATAGAAACCCCGTACCGAGCGCTCGGGAGCAATGTTCCCAGGGACG
ACTCTATCCGTCCGGGGGCCACTCTGCCTCCGTTCGATACCGCAGCACCT
GATTTCGATACAGGTACTTCCCCGACCCCCACTACCGTGCCAGAGCCAGC
CATTACTACACTCATACCGCGCAGCACTAGCGATATGGGATTCTTCTCCA
CGGCACGTGCTACCGGATCAGAAACTCTTTCGGTACCCGTCCAGGAAACG
GATAGAACTCTTTCGACAACTCCTCTTACCCTTCCACTGACTCCCGGTGA
GTCAGAAAATACACTGTTTCCTACGACCGCGCCGGGGATTTCTACCGAGA
CCCCGAGCGCGGCACATGAAACTACACAGACCCAGAGTGCAGAAACGGTG
GTCTTTACTCAGAGTCCGAGTACCGAGTCGGAAACCGCGCGGTCCCAGAG
TCAGGAACCGTGGTATTTTACTCAGACTCCGAGTACTGAACAGGCGGCTC
TTACTCAGACGCAGATCGCAGAAACGGAGGCGTTGTTTACTCAGACTCCG
AGTGCTGAACAGATGACTTTTACTCAGACTCCGGGTGCAGAAACCGAGGC
ACCTGCCCAGACCCCGAGCACGATACCCGAGATATTTACTCAGTCTCGTA
GCACGCCCCCCGAAACCGCTCGCGCTCCGAGCGCGGCGCCGGAGGTTTTT
ACACAGAGTTCGAGTACGGTAACGGAGGTGTTTACTCAGACCCCGAGCAC
GGTACCGAAAACTACTCTGAGTTCGAGTACTGAACCGGCGATTTTTACTC
GGACTCAGAGCGCGGGAACTGAGGCCTTTACTCAGACTTCGAGTGCCGAG
CCGGACACTATGCGAACTCAGAGTACTGAAACACACTTTTTCACTCAGGC
CCCGAGTACGGTACCGAAAGCTACTCAGACTCCGAGTACAGAGCCGGAGG
TGTTGACTCAGAGTCCGAGTACCGAACCTGTGCCTTTCACCCGGACTCTG
GGCGCAGAGCCGGAAATTACTCAGACCCCGAGCGCGGCACCGGAGGTTTA
TACTCGGAGTTCGAGTACGATGCCAGAAACTGCACAGAGCACACCCCTGG
CCTCGCAAAACCCTACCAGTTCGGGAACCGGGACGCATAATACTGAACCG
AGGACTTATCCAGTGCAAACGACACCACATACCCAGAAACTCTACACAGA
AAATAAGACTTTATCGTTTCCTACTGTTGTTTCAGAATTCCATGAGATGT
CGACGGCAGAGTCGCAGACGCCCCTATTGGACGTCAAAATTGTAGAGGTG
AAGTTTTCAAACGATGGCGAAGTAACGGCGACTTGCGTTTCCACCGTCAA
ATCTCCCTATAGGGTAGAAACTAATTGGAAAGTAGACCTCGTAGATGTAA
TGGATGAAATTTCTGGGAACAGTCCCGCCGGGGTTTTTAACAGTAATGAG
AAATGGCAGAAACAGCTGTACTACAGAGTAACCGATGGAAGAACATCGGT
CCAGCTAATGTGCCTGTCGTGCACGAGCCATTCTCCGGAACCTTACTGTC
TTTTCGACACGTCTCTTATAGCGAGGGAAAAAGATATCGCGCCAGAGTTA
TACTTTACCTCTGATCCGCAAACGGCATACTGCACAATAACTCTGCCGTC
CGGCGTTGTTCCGAGATTCGAATGGAGCCTTAATAATGTTTCACTGCCGG
AATATTTGACGGCCACGACCGTTGTTTCGCATACCGCTGGCCAAAGTACA
GTGTGGAAGAGCAGCGCGAGAGCAGGCGAGGCGTGGATTTCTGGCCGGGG
AGGCAATATATACGAATGCACCGTCCTCATCTCAGACGGCACTCGCGTTA
CTACGCGAAAGGAGAGGTGCTTAACAAACACATGGATTGCGGTGGAAAAC
GGTGCTGCTCAGGCGCAGCTGTATTCACTCTTTTCTGGACTTGTGTCAGG
ATTATGCGGGAGCATATCTGCTTTGTACGCAACGCTATGGACCGCCATTT
ATTTTTGAGGAATGCTTTTTGGACTATCGTACTGCTTTCTTCCTTCGCTA
GCCAGAGCACCGCCGCCGTCACGTACGACTACATTTTAGGCCGTCGCGCG
CTCGACGCGCTAACCATACCGGCGGTTGGCCCGTATAACAGATACCTCAC
TAGGGTATCAAGAGGCTGCGACGTTGTCGAGCTCAACCCGATTTCTAACG
TGGACGACATGATATCGGCGGCCAAAGAAAAAGAGAAGGGGGGCCCTTTC
GAGGCCTCCGTCGTCTGGTTCTACGTGATTAAGGGCGACGACGGCGAGGA
CAAGTACTGTCCAATCTATAGAAAAGAGTACAGGGAATGTGGCGACGTAC
AACTGCTATCTGAATGCGCCGTTCAATCTGCACAGATGTGGGCAGTGGAC
TATGTTCCTAGCACCCTTGTATCGCGAAATGGCGCGGGACTGACTATATT
CTCCCCCACTGCTGCGCTCTCTGGCCAATACTTGCTGACCCTGAAAATCG
GGAGATTTGCGCAAACAGCTCTCGTAACTCTAGAAGTTAACGATCGCTGT
TTAAAGATCGGGTCGCAGCTTAACTTTTTACCGTCGAAATGCTGGACAAC
AGAACAGTATCAGACTGGATTTCAAGGCGAACACCTTTATCCGATCGCAG
ACACCAATACACGACACGCGGACGACGTATATCGGGGATACGAAGATATT
CTGCAGCGCTGGAATAATTTGCTGAGGAAAAAGAATCCTAGCGCGCCAGA
CCCTCGTCCAGATAGCGTCCCGCAAGAAATTCCCGCTGTAACCAAGAAAG
CGGAAGGGCGCACCCCGGACGCAGAAAGCAGCGAAAAGAAGGCCCCTCCA
GAAGACTCGGAGGACGACATGCAGGCAGAGGCTTCTGGAGAAAATCCTGC
CGCCCTCCCCGAAGACGACGAAGTCCCCGAGGACACCGAGCACGATGATC
CAAACTCGGATCCTGACTATTACAATGACATGCCCGCCGTGATCCCGGTG
GAGGAGACTACTAAAAGTTCTAATGCCGTCTCCATGCCCATATTCGCGGC
GTTCGTAGCCTGCGCGGTCGCGCTCGTGGGGCTACTGGTTTGGAGCATCG
TAAAATGCGCGCGTAGCTAATCGAGCCTAGAATAGGTGGTTTCTTCCTAC
ATGCCACGCCTCACGCTCATAATATAAATCACATGGAATAGCATACCAAT
GCCTATTCATTGGGACGTTCGAAAAGCATGGCATCGCTACTTGGAACTCT
GGCTCTCCTTGCCGCGACGCTCGCACCCTTCGGCGCGATGGGAATCGTGA
TCACTGGAAATCACGTCTCCGCCAGGATTGACGACGATCACATCGTGATC
GTCGCGCCTCGCCCCGAAGCTACAATTCAACTGCAGCTATTTTTCATGCC
TGGCCAGAGACCCCACAAACCCTACTCAGGAACCGTCCGCGTCGCGTTTC
GGTCTGATATAACAAACCAGTGCTACCAGGAACTTAGCGAGGAGCGCTTT
GAAAATTGCACTCATCGATCGTCTTCTGTTTTTGTCGGCTGTAAAGTGAC
CGAGTACACGTTCTCCGCCTCGAACAGACTAACCGGACCTCCACACCCGT
TTAAGCTCACTATACGAAATCCTCGTCCGAACGACAGCGGGATGTTCTAC
GTAATTGTTCGGCTAGACGACACCAAAGAACCCATTGACGTCTTCGCGAT
CCAACTATCGGTGTATCAATTCGCGAACACCGCCGCGACTCGCGGACTCT
ATTCCAAGGCTTCGTGTCGCACCTTCGGATTACCTACCGTCCAACTTGAG
GCCTATCTCAGGACCGAGGAAAGTTGGCGCAACTGGCAAGCGTACGTTGC
CACGGAGGCCACGACGACCAGCGCCGAGGCGACAACCCCGACGCCCGTCA
CTGCAACCAGCGCCTCCGAACTTGAAGCGGAACACTTTACCTTTCCCTGG
CTAGAAAATGGCGTGGATCATTACGAACCGACACCCGCAAACGAAAATTC
AAACGTTACTGTCCGTCTCGGGACAATGAGCCCTACGCTAATTGGGGTAA
CCGTGGCTGCCGTCGTGAGCGCAACGATCGGCCTCGTCATTGTAATTTCC
ATCGTCACCAGAAACATGTGCACCCCGCACCGAAAATTAGACACGGTCTC
GCAAGACGACGAAGAACGTTCCCAAACTAGAAGGGAATCGCGAAAATTTG
GACCCATGGTTGCGTGCGAAATAAACAAGGGGGCTGACCAGGATAGTGAA
CTTGTGGAACTGGTTGCGATTGTTAACCCGTCTGCGCTAAGCTCGCCCGA
CTCAATAAAAATGTGA (SEQ ID NO: 4, residues
129739-135004 of NCBI reference sequence
number NC_006623.1 which is incorporated
by reference in its entirety.)

The sequence provided below is a non-limiting example of a fully deoptimized version of SEQ ID NO: 4. Deoptimization was performed on the ORF according to methods of the present disclosure. The codon pair score of the following sequence was calculated to be −0.8685.

ATGGGGACGATGCTAGTCTTGCGCCTATTCCTACTAGCAGTAGCGGACGCAG
CGCTACCGACCGGTCGGTTTTGCCGAGTTTGGAAGGTGCCGCCAGGGGGAACGATC
CAAGAGAATCTGGCGGTACTCGCGGAATCGCCGGTAACCGGTCATGCGACATATCC
GCCGCCGGAAGGCGCGGTATCGTTCCAAATTTTTGCGGATACCCCTACGTTGCGCAT
TCGGTACGGCGCGACCGAAGACGAACTAGCGCTCGAACGCGGGACGAGTGCGTCCG
ACGCGGATAACGTAACGTTTTCGCTATCATATCGCCCGCGCCCAGAAATACACGGTG
CATACTTTACGATAGGGGTATTCGCTACCGGCCAAAGCACCGAATCGTCGTATTCGG
TAATCTCGCGGGTATTAGTAAACGCGTCACTGGAACGGTCCGTGCGCCTGGAAACGC
CTTGCGATGAAAACTTTCTCCAAAACGAACCGACTTGGGGTTCGAAGCGGTGGTTAG
GGCCCCCGAGTCCTTACGTGCGCGATAACGACGTAGCCGTGTTGACAAAAGCGCAG
TATATAGGCGAATGCTACTCGAATTCGGCGGCCCAAACGGGTCTAACGTCGCTAAAT
ATGACGTTTTTCTATTCGCCTAAACGTATCGTAAACGTCACGTGGACAACCGGAGGT
CCGTCCCCGTCGCGCATAACGGTATATTCGTCGCGGGAGAACGGTCAGCCGGTATTG
AGGAACGTTTCTGACGGATTCCTAGTTAAGTATACGCCCGATATTGACGGCAGGGCG
ATGATAAACGTTATCGCGAATTATTCGCCAGCGGACTCGGGTAGCGTCCTCGCGTTT
ACGGCGTTTCGCGAAGGTAAGTTACCAAGTGCGATACAATTACACCGTATCGATATG
TCCGGGACTGAGCCGCCGGGGACTGAAACGACCTTCGATTGTCAAAAAATGATCGA
AACCCCGTATAGGGCGCTAGGGAGCAATGTTCCGCGAGACGACTCTATACGTCCGG
GTGCCACTCTACCTCCATTCGATACGGCAGCACCGGATTTCGATACAGGTACTTCCC
CGACTCCCACTACCGTACCGGAGCCAGCCATTACTACCCTAATACCGCGATCGACTT
CCGATATGGGATTCTTCTCCACCGCTCGTGCTACCGGTTCAGAAACTCTATCGGTAC
CCGTACAGGAAACGGATCGTACTCTTTCGACAACTCCTTTAACCCTTCCGCTGACGC
CTGGTGAATCAGAAAATACGCTATTTCCTACGACCGCGCCGGGTATATCTACCGAGA
CCCCGAGCGCGGCACATGAAACTACACAAACCCAAAGTGCAGAAACGGTCGTCTTT
ACTCAAAGTCCGTCGACCGAGTCGGAAACCGCGCGATCCCAGTCGCAAGAACCGTG
GTATTTTACTCAGACGCCGAGTACTGAACAAGCGGCACTAACTCAAACGCAAATAG
CAGAAACGGAAGCGTTGTTTACTCAGACTCCGAGTGCTGAACAAATGACTTTTACTC
AGACTCCCGGTGCGGAAACCGAAGCGCCTGCCCAGACCCCTAGCACGATACCCGAA
ATATTTACTCAGTCTCGTTCGACGCCCCCCGAAACGGCTCGCGCGCCGAGCGCGGCG
CCGGAAGTCTTTACACAGAGTTCGAGTACGGTAACGGAAGTGTTTACTCAGACCCCG
AGCACCGTACCGAAAACTACTCTAAGTTCGAGTACTGAACCGGCGATATTTACTCGG
ACTCAGTCGGCCGGAACTGAAGCCTTTACTCAGACTTCGAGTGCCGAACCGGATACT
ATGCGAACTCAGAGTACTGAAACGCATTTTTTCACGCAGGCGCCATCGACGGTACCG
AAAGCGACTCAGACTCCGAGTACAGAGCCGGAGGTGTTGACTCAGAGTCCGAGTAC
CGAACCGGTGCCGTTTACCCGGACGCTAGGCGCAGAGCCGGAAATTACTCAGACCC
CGAGCGCGGCACCGGAGGTTTATACTCGAAGTTCGTCGACGATGCCAGAAACTGCG
CAGAGCACTCCGTTAGCGTCGCAAAATCCGACTTCGTCGGGTACGGGTACGCATAAT
ACTGAACCGCGTACTTATCCGGTGCAAACGACACCACATACCCAAAAACTATATAC
AGAAAATAAGACGTTATCGTTTCCTACTGTTGTATCGGAATTTCACGAAATGTCGAC
CGCAGAATCGCAGACGCCGCTATTGGACGTAAAGATAGTCGAGGTTAAGTTTTCGA
ACGACGGTGAGGTAACGGCGACATGCGTTTCCACCGTTAAATCGCCCTATAGGGTCG
AAACTAATTGGAAAGTCGACCTAGTCGATGTAATGGACGAAATATCTGGTAATAGT
CCAGCGGGCGTTTTCAATAGTAACGAGAAGTGGCAGAAACAGCTGTATTACAGAGT
AACCGATGGTAGAACGTCGGTACAATTGATGTGTCTATCGTGTACGTCGCATTCTCC
GGAACCGTACTGTCTTTTCGACACGTCTCTAATAGCGAGGGAAAAAGACATAGCGC
CCGAACTATACTTTACGTCTGATCCGCAAACCGCATATTGTACGATAACTCTGCCGT
CCGGAGTCGTTCCGCGATTCGAATGGAGCCTAAATAATGTATCGCTGCCGGAATACC
TAACGGCCACGACCGTTGTTTCGCATACCGCTGGTCAAAGTACCGTATGGAAAAGCA
GTGCGCGTGCGGGCGAAGCGTGGATTTCTGGTAGGGGGGGCAATATATACGAATGT
ACGGTCCTCATATCAGACGGCACGCGCGTAACTACGCGTAAGGAGCGGTGTCTAAC
AAATACGTGGATAGCGGTGGAAAACGGCGCTGCTCAAGCGCAACTGTATTCACTAT
TTTCGGGACTCGTGTCGGGATTATGCGGTAGCATTTCGGCGCTATACGCGACCCTAT
GGACCGCCATATACTTT (SEQ ID NO: 5, deoptimized version of SEQ ID NO: 4
ORF obtained from NCBI reference sequence number NC_006623.1 which is
incorporated by reference in its entirety.)

### Example 5: Codon Pair Deoptimization of ILTV sORF1

Using sequences obtained from NCBI reference sequence number NC_006623.1, located between nucleotides 126616-129615 sORF1 also called UL47 (translocated to unique short region) consists of 3000 nucleotides. The UL47 gene encodes for a major tegument protein. Deletion of the UL47 gene reduced replication 10-fold and significantly attenuated the virus in vivo and reduced amounts of shedding compared to the wild type virus (D. Helferich, et al., “The UL47 gene of avian infectious laryngotracheitis virus is not essential for in vitro replication but is relevant for virulence in chickens”, Journal of General Virology, 88: 732-742 (2007)).

The below sequence shows the wild type sequence of sORF1. The codon pair score for this sequence was calculated to be −0.1638.

ATGACCTTGCCCCATCGATTAACGAAAAGACCTTTCGCGCGTCGATTCTGCTC
GGTCTTTGTGATACATTATAGTGAGACTAAACTCGACCGATATAACAAGACAATGTT
ACTCTATAGACCGGACTCAACCATGCGGCATAGCGGAGGCGACGCAAATCACAGAG
GGATAAGGCCGAGGCGGAAATCTATTGGAGCGTTTAGCGCGCGCGAAAAGACTGGA
AAACGAAATGCGCTGACGGAAAGCAGCTCCTCCTCCGACATGCTAGATCCGTTTTCC
ACGGATAAGGAATTTGGCGGTAAGTGGACGGTAGACGGACCTGCCGACATTACTGC
CGAGGTCCTTTCTCAGGCATGGGACGTTCTCCAATTAGTGAAGCATGAAGATGCGGA
GGAGGAGAGAGTGACTTATGAGTCCAAACCGACCCCGATACAGCCGTTCAATGCCT
GGCCGGACGGGCCGAGTTGGAACGCGCAGGATTTTACTCGAGCGCCAATAGTTTAT
CCCTCTGCGGAGGTATTGGACGCAGAGGCGTTGAAAGTAGGGGCATTCGTTAGCCG
AGTTTTACAATGTGTACCGTTCACGCGATCAAAGAAAAGCGTTACGGTGCGGGATGC
GCAGTCGTTTTTGGGGGACTCGTTCTGGAGAATAATGCAGAACGTTTACACGGTTGT
CTTACGACAGCACATAACTCGACTCAGGCACCCTTCCAGCAAAAGCATTGTTAACTG
CAACGACCCTCTATGGTACGCCTACGCGAATCAATTTCACTGGAGAGGAATGCGCGT
GCCGTCGCTTAAATTAGCCTCTCCCCCGGAGGAGAATATTCAACACGGCCCAATGGC
CGCCGTTTTTAGAAACGCGGGGGCTGGTCTGTTCCTGTGGCCTGCCATGCGCGCAGC
CTTTGAAGAGCGCGACAAGCGACTGTTAAGAGCATGCCTGTCTTCACTCGATATCAT
GGACGCAGCCGTCCTCGCGTCGTTTCCATTTTACTGGCGCGGCGTCCAAGACACCTC
GCGCTTCGAGCCTGCGCTGGGCTGTTTGTCAGAGTACTTTGCACTAGTGGTGTTACT
GGCCGAGACGGTCTTAGCGACCATGTTCGACCACGCACTGGTATTCATGAGGGCGCT
GGCAGACGGCAATTTCGATGACTATGACGAAACTAGATATATAGACCCCGTTAAAA
ACGAGTACCTGAACGGAGCCGAGGGTACTCTGTTACGGGGCATAGTGGCCTCCAAC
ACCGCTCTGGCGGTGGTTTGCGCAAACACCTATTCGACGATAAGAAAACTCCCGTCC
GTGGCAACTAGCGCGTGCAATGTTGCCTACAGGACCGAAACGCTGAAAGCGAGGCG
CCCTGGCATGAGCGACATATACCGGATATTACAAAAAGAGTTTTTCTTTTACATTGC
GTGGCTCCAGAGGGTTGCAACACACGCAAATTTCTGTTTAAACATTCTGAAGAGAAG
CGTGGATACGGGGGCCCCGCCATTTTTGTTCAGGGCCAGCTCGGAGAAGCGGCTGC
AGCAGTTAAATAAAATGCTCTGCCCCCTTCTCGTGCCGATTCAATATGAAGACTTTT
CGAAGGCCATGGGGTCTGAGCTCAAGAGGGAAAAGTTAGAGACATTCGTTAAAGCT
ATTTCCAGCGACAGGGACCCGAGGGGGTCCTTAAGATTTCTCATTTCGGACCATGCA
AGGGAAATTATTGCAGACGGAGTACGGTTTAAGCCGGTGATAGACGAGCCGGTTCG
GGCTTCAGTTGCGCTGAGTACCGCTGCCGCTGGGAAAGTGAAAGCGCGACGCTTAA
CCTCAGTTCGCGCGCCCGTACCGGGCGCAGGCGCCGTTTCCGCGCGCCGGAAATCG
GAAATATGATAAAAATGCTTGGCATTTGCGGGCGAAGAGGCGTGATCTGAAGGGCT
CCACAATGACGTAACTGAGCTACGCATCCCTATAAAGTGTACCCGCTGACCGCTAGC
CCATACAGTGTTACAGGAGGGGAGAGAGACAACTTCAGCTCGAAGTCTGAAGAGAC
ATCATGAGCGGCTTCAGTAACATAGGATCGATTGCCACCGTTTCCCTAGTATGCTCG
CTTTTGTGCGCATCTGTATTAGGGGCGCCGGTACTGGACGGGCTCGAGTCGAGCCCT
TTCCCGTTCGGGGGCAAAATTATAGCCCAGGCGTGCAACCGCACCACGATTGAGGT
GACGGTCCCGTGGAGCGACTACTCTGGTCGCACCGAAGGAGTGTCAGTCGAGGTGA
AATGGTTCTACGGGAATAGTAATCCCGAAAGCTTCGTGTTCGGGGTGGATAGCGAA
ACGGGCAGTGGACACGAGGACCTGTCTACGTGCTGGGCTCTAATCCATAATCTGAAC
GCGTCTGTGTGCAGGGCGTCTGACGCCGGGATACCTGATTTCGACAAGCAGTGCGA
AAAAGTGCAGAGAAGACTGCGCTCCGGGGTGGAACTTGGTAGTTACGTGTCTGGCA
ATGGATCCCTGGTGCTGTACCCAGGGATGTACGATGCCGGCATCTACGCCTACCAGC
TCTCAGTGGGTGGGAAGGGATATACCGGGTCTGTTTATCTAGACGTCGGACCAAACC
CCGGATGCCACGACCAGTATGGGTACACCTATTACAGCCTGGCCGACGAGGCGTCA
GACTTATCATCTTATGACGTAGCCTCGCCCGAACTCGACGGTCCTATGGAGGAAGAT
TATTCCAATTGTCTAGACATGCCCCCGCTACGCCCATGGACAACCGTTTGTTCGCAT
GACGTCGAGGAGCAGGAAAACGCCACGGACGAGCTTTACCTATGGGACGAGGAATG
CGCCGGTCCGCTGGACGAGTACGTCGACGAAAGGTCAGAGACGATGCCCAGGATGG
TTGTCTTTTCACCGCCCTCTACGCTCCAGCAGTAGCCACCCGAGAGTGTTTTTTGTGA
GCGCCCACGCAACATACCTAACTGCTTCATTTCTGATCAATTATTGCGTATTGAATA
AATAAA (SEQ ID NO: 6, residues 126616-129615 of NCBI reference sequence
number NC_006623.1 which is incorporated by reference in its entirety.)

The sequence provided below is a non-limiting example of a fully deoptimized version of SEQ ID NO: 6. Deoptimization was performed on the ORF according to methods of the present disclosure. The codon pair score of the following sequence was calculated to be −0.8071.

ATGACGCTACCGCATCGCCTAACGAAACGACCTTTCGCGCGTCGATTCTGTTC
GGTATTCGTAATACACTATTCGGAAACTAAACTCGATCGGTATAATAAGACGATGCT
ACTCTATCGACCGGACTCAACGATGCGTCATTCGGGTGGCGACGCAAATCATAGGG
GTATACGACCGCGACGGAAGTCGATTGGTGCGTTTTCGGCACGCGAAAAAACTGGT
AAGCGTAATGCGCTAACCGAAAGCAGCTCGTCGTCCGATATGCTAGATCCGTTTTCG
ACCGATAAGGAATTCGGGGGTAAGTGGACGGTCGACGGTCCGGCCGATATAACCGC
GGAGGTCCTATCGCAAGCGTGGGACGTTCTCCAACTCGTAAAACATGAAGACGCGG
AAGAGGAACGCGTGACGTACGAATCGAAACCGACTCCGATACAACCGTTTAATGCC
TGGCCGGACGGGCCGAGTTGGAACGCGCAGGACTTTACGCGAGCGCCAATCGTATA
TCCGTCTGCGGAAGTACTCGACGCAGAAGCGCTTAAGGTCGGGGCGTTCGTAAGCC
GCGTTTTACAATGTGTACCGTTCACGCGATCGAAGAAATCGGTTACGGTGCGCGATG
CGCAATCGTTTCTAGGGGATTCGTTTTGGCGTATAATGCAAAACGTATATACGGTCG
TCTTACGCCAACACATTACTCGATTGCGGCATCCGAGTTCGAAATCGATCGTAAATT
GTAACGACCCGTTATGGTATGCGTACGCGAATCAATTTCATTGGAGAGGTATGCGCG
TGCCGTCGCTAAAACTAGCGAGTCCGCCGGAGGAGAATATACAACACGGCCCGATG
GCCGCCGTATTTCGTAACGCGGGTGCGGGTCTATTCCTATGGCCGGCCATGCGTGCA
GCATTCGAAGAGCGCGATAAACGACTGTTACGCGCATGCCTATCGTCACTAGACATA
ATGGACGCAGCCGTCCTCGCGTCGTTTCCATTTTATTGGCGAGGCGTACAAGATACG
TCGCGCTTCGAACCGGCGCTAGGGTGTCTATCGGAATACTTCGCACTAGTCGTGCTC
CTAGCCGAGACGGTATTAGCGACTATGTTCGACCATGCGCTGGTCTTTATGAGGGCG
CTGGCGGACGGTAATTTCGATGATTACGACGAAACGCGATATATAGACCCGGTTAA
GAACGAATACCTAAACGGTGCCGAAGGTACGCTATTACGCGGTATAGTAGCGTCGA
ACACCGCGCTAGCGGTGGTTTGCGCGAATACGTATTCGACGATACGTAAGTTACCGT
CCGTAGCAACTAGCGCGTGTAATGTAGCGTATCGTACGGAAACGCTGAAGGCGAGG
CGACCGGGCATGTCCGACATATACCGTATATTACAAAAAGAGTTTTTTTTCTATATA
GCGTGGTTACAACGCGTTGCGACACATGCAAATTTTTGCCTAAACATTCTGAAAAGA
TCGGTCGATACGGGGGCGCCACCGTTTTTGTTCCGCGCCAGCTCGGAAAAACGCCTA
CAACAATTGAATAAGATGCTATGCCCGTTACTCGTACCGATTCAATACGAAGACTTT
TCGAAGGCGATGGGTTCTGAACTCAAAAGGGAAAAATTAGAGACATTCGTTAAAGC
TATATCGAGCGATCGCGATCCGCGAGGTTCCTTACGGTTTCTAATCTCGGATCATGC
GCGCGAAATTATAGCAGACGGCGTACGGTTTAAACCGGTAATCGACGAGCCGGTTC
GGGCATCGGTTGCGCTAAGTACCGCGGCTGCGGGGAAGGTAAAAGCGCGACGATTG
ACGTCGGTTCGCGCGCCGGTACCGGGTGCAGGTGCGGTATCCGCGCGCCGTAAATC
GGAAATC (SEQ ID NO: 7, deoptimized version of SEQ ID NO: 6 ORF obtained
from NCBI reference sequence number NC_006623.1 which is incorporated
by reference in its entirety.)

### Example 6: Codon Pair Deoptimization of ILTV Glycoprotein C Gene

Using sequences obtained from NCBI reference sequence number NC_006623.1, located between nucleotides 75683-76942 the glycoprotein C gene (UL44) consists of 1260 nucleotides. Glycoprotein C is an envelope protein that is abundant on the virus surface. Deletion of the gC displays delayed penetration kinetics and slightly reduced plaque sizes in vitro, whereas virus titers were not reduced significantly compared with wild type. In vivo, the gC deletion mutant had 20% mortality which was reduced from the mortality of 50% associated with the wild type (Pavlova, S. P., et al., “In vitro and in vivo characterization of glycoprotein C-deleted infectious laryngotracheitis virus”, Journal of General Virology, 91: 847-857 (2010)).

The below sequence shows the wild type sequence of the glycoprotein C gene (UL44). The codon pair score for this sequence was calculated to be −0.0391.

ATGCAGCATCAGAGTACTGCGCTAGTTTCGAGTATACTTTTGCTCTTGAGCCT
GCAAAGCCTTGCGTTTGAATTTTTCTGTGATCCGCCACACGTTTTTCGAGGGCAGCTC
GGTGACCCCATTCTATTGCAATGCTTCAGCGACAGACCTCTAACCCACGAAGAATCT
GTAAAAGTAGAAGTAATTCGACACCCAGCCAGCTTAGTTGAAACTGCGCTAAGCGC
CTACGGGATCCCCCCTTCGCTAGATCCATGGAGAGCTACTCCAAGAACTCTCTACAC
ATATGATGCCGCTACTGATTCAATCAAGGACCTAGGATACATTGGTGAAGATGGAAT
TAACCCACCATATTTGGACGACTGTCGTTCAGGTTTTTTCAATGTCTCTATCAAGTCT
AGCATGAGATCTCACATGGCGCGTTATCAGTGGACCGCAAGTCGAGGGTCTACAAA
ACTAAATAGCTCTTTTATCGACGTCTTTTTGGCAAGACCACCTACAACTGTCCGCATC
AAATCAGAAGAACTGTACGAAGACTCAGATAAGGCTTCGCACTTAAGTGTTGAAGC
GCTTGGCGCTTATCCTCCATCTGCTGCGCTGGGTACATGGATGATACATAATGCATC
TCTTGCTGAAAAATACAGTTTAGAAAGAAGAGTTCTTTATGCATCAGGAGAGAATG
GATCGGTGGATCAGACATGGGAACTGGAAATACGTGGAGAAGCCAGCCAGCCCCTC
CCTTCCAAAATTCAATTTGTATATCGATGGACCCCTCCTGAGGACTTTGAAATGCTA
CGACCTGAAACTCGCTTGTTAAGGTTGACTCCCAGCTGGATTAGCAAGCCCCGCATC
ACGGTACAATTCGTCCCTCCTGCCTATGCCCTGTGTAGAGCAGCTAATATTATAGAC
GGCCGAGGATTTATTGAATGGATCGTAGATAATAGAATTTCGACGAGCCCACACCA
GACCTTTGTTTTGGATGAGCCCGAGGGGAAAAATATCGTTACACTAATGGACGTCAT
AAAACTACCACCGGAGGATACATTTCAATCTGCCTCTAATTACGTGTGCGTCATAAG
AGGCTATGAACATGCATACAGATATCTCAACGCCTCCTTAATGATAGATAATCTGCC
AATGCGGCAAGGATTCCCCGCAGTCGCTGCGATTTTTATTATAATTAGTATCGCTTTT
GTGGGTGGGTTACTAGTTGCTTGCTTGGGCGCATGGTGCTGGAAGACAACATAAACG
CTCATTTAATAA (SEQ ID NO: 8, residues 75683-76942 of NCBI reference
sequence number NC 006623.1 which is incorporated by reference in its
entirety.)

The sequence provided below is a non-limiting example of a fully deoptimized version of SEQ ID NO: 8. Deoptimization was performed on the ORF according to methods of the present disclosure. The codon pair score of the following sequence was calculated to be −0.7881.

ATGCAACACCAATCGACTGCGCTAGTTTCGTCGATCCTTCTACTACTATCGCT
ACAAAGCCTAGCGTTCGAATTCTTTTGTGATCCGCCACACGTATTTCGCGGTCAACT
AGGCGACCCGATACTACTCCAATGTTTTTCGGATCGACCGTTAACGCACGAAGAATC
GGTTAAGGTCGAGGTAATACGCCATCCGGCTTCGTTAGTTGAAACCGCGCTATCGGC
ATACGGGATCCCCCCTAGTCTCGATCCGTGGCGTGCGACTCCGCGTACGCTATATAC
GTACGATGCAGCGACCGATTCAATTAAGGACCTAGGGTATATAGGCGAAGACGGAA
TAAATCCGCCGTATCTAGACGATTGTCGATCGGGTTTTTTTAACGTATCGATCAAGTC
GAGCATGCGATCGCATATGGCGCGATATCAATGGACCGCGTCACGCGGGTCTACGA
AATTGAATTCGTCTTTTATCGACGTATTCCTAGCGCGACCACCTACGACCGTACGCA
TAAAATCGGAAGAGCTGTACGAAGATTCGGATAAGGCGTCGCATCTAAGTGTCGAA
GCGCTTGGCGCGTATCCTCCATCTGCAGCGCTGGGTACGTGGATGATACATAATGCG
TCGCTTGCGGAAAAGTATTCGCTCGAACGTCGCGTACTATACGCGTCAGGCGAAAAC
GGTTCGGTGGACCAAACGTGGGAACTCGAGATACGCGGCGAAGCCTCGCAACCGTT
ACCGTCGAAAATCCAATTCGTATATCGTTGGACGCCTCCTGAAGACTTCGAAATGCT
ACGACCGGAAACGCGACTATTACGCCTAACGCCCAGTTGGATATCGAAGCCGCGTA
TAACGGTACAATTCGTACCGCCAGCCTATGCGCTATGTAGAGCAGCGAATATAATCG
ACGGTAGGGGGTTTATTGAATGGATAGTCGACAATCGGATTTCGACGAGTCCGCATC
AAACCTTCGTACTCGATGAACCGGAAGGTAAGAATATCGTAACGCTTATGGACGTA
ATCAAGTTACCGCCGGAGGATACGTTCCAATCGGCTTCTAATTACGTGTGCGTAATA
AGAGGCTACGAACATGCATATCGGTATCTAAACGCCTCGCTAATGATCGATAACCTA
CCGATGCGTCAAGGGTTTCCGGCGGTCGCTGCGATATTCATAATCATATCGATTGCG
TTTGTAGGCGGACTACTCGTAGCGTGTCTAGGCGCATGGTGTTGGAAAACGACT
(SEQ ID NO: 9, deoptimized version of SEQ ID NO: 8 ORF obtained from
NCBI reference sequence number NC_006623.1 which is incorporated by
reference in its entirety.)

### Example 7: Codon Pair Deoptimization of ILTV UL37 Gene

Using sequences obtained from NCBI reference sequence number NC_006623.1, located between nucleotides 65881-62721 the UL37 gene consists of 3161 nucleotides. UL37 is a tegument protein and in other herpesviruses is an essential neuroinvasion effector. Deoptimization of UL37 reduced the ability of ILTV to go latent in the trigeminal ganglia and reactivate in the trachea (Richards, A. L., et al., “The pUL37 tegument protein guides alpha-herpesvirus retrograde axonal transport to promote neuroinvasion”, PLoS Pathog, 13: 1-32 (2017)).

The below sequence shows the wild type sequence of the UL37 gene. The codon pair score for this sequence was calculated to be −0.0977.

ATGCAGGCCATCACGGATAGCCTCAAGGCTTGCCTTGAAGCTGTACACACCG
ACTGCAGTAAAGTTCCGGGTGTTACTTCAGCCCTCACCGGAATTCTAATCTCCAGAA
ACAGAATTCCACTTGAAGATTTGGAGAAAGTAGAAGCTAGGAAAAACATGGTTGAA
ACTATAATGCTCGCATGCACAATGGCGCCTCCGGCAGTAGCGGACGTTACATTGAAA
TTATTTGCACGCTCTCTTATTAGCCGCATATCTGTTCCATGGAACTCTGGAGAAGATT
TCAGAATAATTCAATATTTGAGAGAAGCATTTACTGGACTGCAACATGACCTAGAAA
CAGCTGTACGTAAAGAATTTCCTGGGTTAAATGATCCAAATGTAGAATATTGGAGTC
TAATTAGTGGCTGGTTAACGGAATTTGGGAGTTTGGCAAGATTGGTAGTTGACGAGC
GACAATTGTTCGAGACATCCGGAGACCGACTCGAGCCAACCAAACTCCTATCTCCTT
TAATTGAAAATTATCCACTTTTATACGACCACCAAATGGTTCAAGACGGGATACAGT
ATCTTGGAACAAAGCTACAAGGCTTAGTGGGATACCATGCAATGTTAAATTATATCA
CGGCTTCAACCGGGCTACCCAAAACTAAAGCGCTGATGACCCTCTCTATGGTCAGAG
AGTATTTTGAACCTACAGTAAAACCCCCTTATCGGAAGTTTCCACAGACGGCGCTAG
CTGCCTTTGATCTGGACACAGATTCGGCCAAGTCATGGATCGCTAAAGATTTAGACT
CAAGATACTTTTCTATCTCGCAACTGTACGCCAATGCTGCATACAGTCGAGACCCAC
TACTCGTGTTAGGAGATCCTCCGAAGACGCATGTTCCTGGACATGTAGTTTGCTGGC
GGCACGACTTAGATAGTCTATTAACTATTCCCATTAAACGCCTCGCCCTAGATGTCC
CAACCATCGTACAGCTTTTCGAGGACGCGCCTAATAACTTGACAGAGGAACAAAAG
GAGACCCTAACTGCTTATACAACAAGTACCGCCGATATGATGGGGCGTTCGCCGGA
CTTGGCCGCTACCGGTGGCCCAGAGGAAGTAATTCGTACTTTAGTGCTTAGGGGGTT
TACCAAGAGCAACTGCGAACTGTATATCCAAAAGGCTGAGGCGCTTTGGGGTGAAG
ATACTGACGGAACAATCATTGGAGATTTCCTAGGAGCCGTTGTATATACATCACTTA
TAGGACTCGCTGCGCACTCAATGTATACATATAATCCGCGGACGCTACAGTATTCAG
GGCATTACGCTGGGCTTATATCTGATTGGGGAGACCCATATTCGCATCTTCTCCGGC
GGGTAGGGGTGTCTGAAAGCGAACTACTACTACCACTACGATCATTTGCCCCAGCCC
CATCAAAAAATGCTCTAAACATCATTAGGGACACTCTGGCCTCATTTATAGATGGGA
GCGATGGAGGACCCGAGGCGCTTGGAAATTTGGGGGTGTTCAATTTTGTTCAACACC
TCATATCTGACTTAGGAGGAAAGACCCAGGAAAATTTGGATTGGCTCGAACGCAGG
GAAAAGGCCAAAGATAAGGGGTATGCAAGTGCCGACAAACAATCCTACACTTATGG
ATTTGACCAAACCGTAAAATTAGTCCAGGCCATTGAAGTTGGGGGCGCGGAGCCTG
AAGAGATATGTTCGCGTAATGGAACGGACCCGAAAATCGATATGCAACTCTTTTCCA
ATCTTTCCGTAGCGACCGTATTGAGAGACATTGTTTACGCAGTATCAACTTATCCATT
CACCGTTGACGCGGTACAACACGCGGTAAAGCTTTTATCGGGGATAGAGATTAGGG
CTCTCAGAAAACCGTCTGATACTCAGAAGTACAGGCGAGCAATTTTGGAATTTCAGT
TAGCGGTCTCCCCATTTATTACAAAGACCAATCCGACAGAAAAGATATCCATCGAAA
GTGCAAGCGCGATAGAAAAGGCATTGCTTAAAATTTCTGACGCATGCGACAGGAGC
CTCGACACTCTTCCAGAAACATTTAAGAAAACAGTCCAGCCTGTCCCTGCCGTCGAT
ACCATGAGATCAAAGTTCATTTCCCAGGCATTCGAGCAAGCTGCAACGAGAGACCTT
GAGGAGGTTTCCAAGACTCTAAATAACGCATCTATGCAGATATCTACTGCAGTGCAA
ATACTACTTTCTAAGACTGCTAACATAAGACCACTGCTTTCACAGTCAGCAAATGCG
GATTTTTTGGGCAATGTTTCTTTCTCAAGTTGCCGTGACAAAAAAGAACTAGGACAC
TGGAAGGACGATACTGTAATGCAGGCCATATGGAAAGCCGTCAATTTTGCAGAAAC
TGCAATCCGACACACAGAAAAAGCTATTCGGGCATTAGATTCCACAAAGGTTAAAA
CGCATGCACTTGAGACCACATACTGGCAGCTTGCGGGTTATGCTCAAACACGCTCTC
ACATTTGTAGGAAAGTAGCTGAAGAATTATCTGGCATAGCGGACGCGAGGAATACT
GTCGTCCAGCGTTCGTGGACATTATGTCGTTCACTCGCAGGCGTCGGGGTAAAAAGG
GCCCGCGATGTGGTAAAAGCCTGGAAAACTTTTTCAGATAATGAAGCTGCTCTCGAG
ACATCTTAATCCAAAACCCTGAAGATGTAGTTAAAGTTATTGGCGCGGCATCAGCTC
TGCTAGATATAGAGGAGGGAGGGACCACGACCCTTGAAGGAAAATTGGAAGAAATT
GAAATTTCTGCCCCTTCTAAAGAAGATACAGCGTCAGCTCTTACTCTTTTAGGGCCG
CCTGATGCCAGGGGATATCAAGATACATCATCAAATCAATTTATTACCGAGGCGTCA
GACATTACTTCGTGGGATAAAACCAACAAAGCCCCGCTCTATGTTCGACACTCGACA
AAAGAAGGGATAGACAATGGGTGTGTAGACACACTTCCAGACAAACTAATCTCATC
AGAGACTCTTCTGACAGTTTCCGATCGAGTTTTGGAAAGTGTAAATGTAAAAATGTT
TGAATAAAGAAAGACTAATGAGTAATTGAGTGGTGTGTATTTATCGCGTTGTTGTGT
GCGGTTGGCTAGACATTTACGAAACTCTTCCCAAACAAATAAA (SEQ ID NO: 10,
residues 65881-62721 of NCBI reference sequence number NC_006623.1
which is incorporated by reference in its entirety.)

The sequence provided below is a non-limiting example of a fully deoptimized version of SEQ ID NO: 10. Deoptimization was performed on the ORF according to methods of the present disclosure. The codon pair score of the following sequence was calculated to be −0.8183.

ATGCAAGCGATCACGGATTCGCTAAAGGCGTGCCTTGAAGCTGTACACACCG
ATTGTTCGAAGGTTCCGGGTGTAACGAGTGCCCTCACCGGTATACTCATATCCAGAA
ACCGGATTCCGTTAGAAGATTTGGAAAAGGTAGAAGCTCGTAAGAATATGGTTGAA
ACTATTATGCTAGCGTGTACGATGGCGCCTCCGGCAGTAGCGGACGTTACGTTGAAA
TTATTTGCGCGATCTCTTATCAGCCGTATATCGGTACCGTGGAATTCTGGCGAAGAC
TTTCGCATAATCCAATATTTGAGAGAAGCATTTACGGGTCTGCAACATGACCTAGAA
ACAGCGGTACGCAAAGAGTTTCCTGGTCTAAACGATCCAAACGTCGAATATTGGAG
TCTAATTTCGGGTTGGTTAACGGAATTCGGTAGTCTAGCGCGACTCGTAGTTGACGA
ACGCCAACTATTCGAAACATCCGGAGATCGACTCGAGCCGACCAAACTCCTATCGC
CGTTAATTGAAAACTATCCACTTCTATACGATCACCAAATGGTTCAAGACGGGATAC
AATATCTCGGTACGAAGCTCCAAGGGTTAGTGGGGTATCATGCGATGCTAAATTATA
TCACCGCTTCGACCGGTCTACCCAAAACGAAAGCGCTTATGACGCTATCGATGGTCA
GAGAATATTTCGAACCTACAGTTAAGCCGCCTTATCGTAAGTTTCCACAAACCGCGC
TAGCGGCATTTGATCTGGACACGGATAGTGCCAAGTCATGGATCGCTAAAGATTTAG
ATTCACGATATTTTTCTATCTCGCAACTGTACGCCAATGCTGCATACAGTCGCGATCC
ACTACTCGTACTAGGAGATCCGCCGAAGACGCACGTACCTGGTCATGTCGTATGTTG
GCGACACGACCTCGATAGTCTATTAACGATACCGATTAAACGCCTAGCGCTAGATGT
CCCGACCATCGTACAACTTTTCGAGGACGCACCGAATAACTTGACCGAAGAGCAAA
AGGAAACGCTAACTGCTTATACGACTAGTACCGCGGATATGATGGGTCGGAGTCCG
GACTTGGCGGCTACGGGCGGCCCAGAGGAGGTAATTCGTACGCTAGTCCTTCGCGGT
TTTACGAAGTCGAATTGCGAACTATATATACAAAAAGCTGAAGCGCTATGGGGTGA
AGATACTGACGGTACAATCATAGGCGATTTCCTCGGAGCCGTCGTATATACGTCACT
AATAGGACTAGCTGCACATTCGATGTATACGTATAATCCGCGTACGCTACAGTATTC
GGGTCATTATGCGGGTCTTATATCTGATTGGGGCGACCCGTATTCGCATCTATTGCGT
CGGGTAGGCGTATCGGAATCGGAACTACTACTACCATTACGATCGTTCGCGCCAGCC
CCATCAAAAAACGCTCTAAACATAATAAGGGACACGCTGGCGTCGTTTATCGACGG
TTCGGACGGGGGTCCCGAGGCGCTAGGTAACTTGGGGGTATTCAATTTCGTTCAACA
CCTAATCTCGGACCTAGGCGGAAAGACCCAAGAAAATTTGGATTGGCTCGAACGTA
GGGAAAAAGCCAAAGATAAGGGGTATGCATCAGCGGATAAGCAATCGTATACTTAC
GGATTCGATCAAACGGTAAAGTTAGTCCAGGCCATTGAAGTCGGGGGTGCGGAACC
TGAAGAGATATGTTCGCGTAACGGTACGGATCCGAAAATCGATATGCAACTCTTTTC
CAACCTATCCGTAGCGACCGTATTAAGAGACATCGTTTACGCGGTATCAACGTATCC
GTTCACGGTCGACGCGGTACAACACGCGGTTAAGCTATTATCGGGGATCGAAATTCG
CGCGCTCAGAAAACCGTCCGATACGCAGAAGTACCGACGTGCGATACTCGAATTCC
AGTTAGCGGTAAGTCCGTTTATTACAAAGACCAATCCGACCGAAAAGATATCGATC
GAATCGGCGAGCGCGATAGAAAAGGCACTACTTAAGATTTCTGACGCATGCGATAG
GAGCCTAGATACTCTACCGGAAACGTTTAAGAAAACGGTCCAGCCTGTACCTGCCGT
CGATACGATGAGATCAAAATTCATTTCCCAAGCGTTCGAACAAGCTGCTACGAGAG
ATCTTGAAGAGGTTTCCAAGACGCTAAACAATGCGTCGATGCAAATATCGACCGCA
GTACAAATCCTACTTTCGAAGACCGCTAATATACGACCGCTGCTATCGCAATCAGCG
AATGCGGATTTTTTGGGTAATGTTTCGTTTTCGTCGTGTCGTGATAAAAAAGAATTA
GGGCATTGGAAGGACGATACGGTAATGCAGGCGATATGGAAAGCCGTAAACTTTGC
GGAAACCGCAATACGCCATACAGAAAAAGCGATTCGGGCACTCGATTCGACAAAAG
TTAAAACGCATGCGCTTGAGACGACGTATTGGCAACTTGCGGGTTATGCGCAAACGC
GATCGCATATATGTCGTAAGGTCGCGGAAGAGCTATCTGGTATAGCGGACGCGAGG
AATACGGTCGTACAGCGATCGTGGACACTATGTCGATCGCTCGCGGGCGTCGGCGTA
AAAAGGGCACGCGACGTAGTCAAAGCCTGGAAAACTTTTTCGGATAATGAAGCGGC
TCTCGAGACGAGT (SEQ ID NO: 11, deoptimized version of SEQ ID NO: 10 ORF
obtained from NCBI reference sequence number NC_006623.1 which is
incorporated by reference in its entirety.)

### Example 8. Codon Pair Optimization of the Newcastle Disease Fusion Gene

Using sequences obtained from GenBank DQ195265.1. The fusion is an envelope protein responsible for entry of virus into the cell by fusion of membranes. When inserted into the herpesvirus genome, the fusion protein alone allows for protection against Newcastle disease. (X. Wei, et al., “Glycoprotein-C-gene-deleted recombinant infectious laryngotracheitis virus expressing a genotype VII Newcastle disease virus fusion protein protects against virulent infectious laryngotracheitis virus and Newcastle disease virus”, Veterinary Microbiology, 250: 1-11 (2020); Esaki, M., et al., “Protection and antibody response caused by turkey herpesvirus vector Newcastle disease vaccine” Avian Diseases, 57: 750-755 (2013)). The Fusion gene will be codon pair optimized based on the Gallus gallus genome. In vitro comparative studies will be conducted showing expression level of codon pair optimized verses codon optimized versions of the fusion gene.

The below sequence shows the wild type sequence of the fusion gene. The codon pair score for this sequence was calculated to be −0.0429.

ATGGGCTCCAGACCTTCTACCAAGAACCCAGCACCTATGACGCTGACTATCC
GGGTTGCGCTGGTACTGAGTTGCATCTGTCCGGCAAACTCCATTGATGGCAGGCCTC
TTGCAGCTGCAGGAATCGTGGTTACAGGAGACAAAGCCGTCAACATATACACCTCA
TCCCAGACAGGATCAATCATAGTTAAGCTCCTCCCGAATCTGCCCAAGGATAAGGA
GGCATGTGCGAAAGCCCCCTTGGATGCATACAACAGGACATTGACCACTTTGCTCAC
CCCCCTTGGTGACTCTATCCGTAGGATACAAGAGTCTGTGACTACATCTGGAGGGGG
GAGACAGGGGCGCCTTATAGGTGCCATTATTGGCGGTGTGGCTCTTGGGGTTGCAAC
TGCCGCACAAATAACAGCGGCCGCAGCTCTGATACAAGCCAAACAAAATGCTGCCA
ACATCCTCCGACTTAAAGAGAGCATTGCCGCAACCAATGAGGCTGTGCATGAGGTC
ACTGACGGATTATCGCAACTAGCAGTGGCGGTTGGGAAGATGCAGCAGTTTGTTAAT
GACCAATTTAATAAAACAGCTCAGGAATTAGACTGCATCAAAATTGCACAGCAAGT
TGGTGTAGAGCTCAACCTGTACCTAACCGAATTGACTACAGTATTCGGACCACAAAT
CACTTCACCTGCTTTAAACAAGCTGACTATTCAGGCACTTTACAATCTAGCTGGTGG
AAATATGGATTACTTATTGACTAAGTTAGGTGTAGGGAACAATCAACTCAGCTCATT
AATCGGTAGCGGCTTAATCACCGGTAACCCTATTCTATACGACTCACAGACTCAACT
CTTGGGTATACAGGTAACTCTACCTTCAGTCGGGAACCTAAATAATATGCGTGCCAC
CTACTTGGAAACCTTATCCGTAAGCACAACCAGGGGATTTGCCTCGGCACTTGTCCC
AAAAGTGGTGACACAGGTCGGTTCTGTGATAGAAGAACTTGACACCTCATACTGTAT
AGAAACTGACTTAGATTTATATTGTACAAGAATAGTAACGTTCCCTATGTCCCCTGG
TATTTATTCCTGCTTGAGCGGCAATACGTCGGCCTGTATGTACTCAAAGACCGAAGG
CGCACTTACTACACCATACATGACTATCAAAGGTTCAGTCATCGCCAACTGCAAGAT
GACAACATGTAGATGTGTAAACCCCCCGGGTATCATATCGCAAAACTATGGAGAAG
CCGTGTCTCTAATAGATAAACAATCATGCAATGTTTTATCCTTAGGCGGGATAACTT
TAAGGCTCAGTGGGGAATTCGATGTAACTTATCAGAAGAATATCTCAATACAAGATT
CTCAAGTAATAATAACAGGCAATCTTGATATCTCAACTGAGCTTGGGAATGTCAACA
ACTCGATCAGTAATGCTTTGAATAAGTTAGAGGAAAGCAACAGAAAACTAGACAAA
GTCAATGTCAAACTGACTAGCACATCTGCTCTCATTACCTATATCGTTTTGACTATCA
TATCTCTTGTTTTTGGTATACTTAGCCTGATTCTAGCATGCTACCTAATGTACAAGCA
AAAGGCGCAACAAAAGACCTTATTATGGCTTGGGAATAATACTCTAGATCAGATGA
GAGCCACTACAAAAATGTGA (SEQ ID NO: 12, residues 1 to 1662 of NCBI
reference sequence number DQ195265.1 which is incorporated by reference
in its entirety.)

The sequence provided below is a non-limiting example of a fully optimized version of SEQ ID NO: 12. Optimization was performed on the ORF according to methods of the present disclosure. The codon pair score of the following sequence was calculated to be 0.4418.

ATGGGCAGCCGCCCTTCTACCAAGAACCCAGCACCAATGACGCTGACCATCCGGGTTGCCCTGGTACTGAGCTGCAT
CTGCCCAGCCAACAGCATTGATGGCCGGCCTCTGGCAGCAGCAGGAATTGTGGTGACAGGAGATAAAGCCGTGAACA
TCTACACCTCAAGCCAGACAGGCAGCATCATAGTGAAGCTGCTCCCGAACCTGCCCAAGGACAAAGAAGCCTGTGCC
AAAGCTCCCCTGGATGCCTACAACAGAACATTGACCACTCTGCTGACACCACTGGGAGACAGCATCCGCAGGATCCA
GGAGTCTGTGACCACCAGTGGAGGAGGAAGGCAGGGCCGCCTGATTGGAGCCATCATTGGCGGTGTGGCTCTTGGAG
TTGCAACTGCCGCACAGATAACAGCAGCCGCAGCACTGATACAGGCCAAACAGAATGCTGCCAACATCCTGCGGCTG
AAAGAGAGCATTGCCGCCACCAATGAGGCTGTGCATGAAGTGACAGATGGGCTGAGCCAGCTAGCAGTGGCTGTGGG
GAAGATGCAGCAGTTTGTGAATGACCAGTTTAACAAAACAGCACAGGAACTGGACTGCATCAAGATTGCACAGCAAG
TGGGTGTGGAGCTGAACCTGTACCTGACCGAGCTGACAACAGTATTTGGACCACAAATCACCTCTCCAGCTTTAAAC
AAGCTGACTATCCAGGCACTGTACAACCTGGCAGGTGGAAACATGGACTACCTGTTGACCAAGCTGGGAGTAGGCAA
CAACCAGCTGAGCAGCTTAATTGGCAGCGGCTTAATCACCGGAAACCCCATCCTGTATGACTCACAGACACAGCTCC
TGGGCATCCAGGTGACTCTGCCTTCTGTCGGGAACCTAAATAACATGCGTGCCACCTACCTGGAAACACTGTCTGTC
AGCACAACCAGAGGATTTGCCTCGGCACTGGTGCCCAAAGTGGTGACACAGGTCGGTAGCGTGATTGAAGAGCTTGA
CACCTCCTACTGTATTGAAACAGACCTGGACTTATACTGCACAAGAATTGTCACCTTCCCCATGAGCCCCGGCATTT
ACAGCTGCCTGAGCGGCAACACATCGGCCTGCATGTACAGCAAAACCGAAGGAGCACTTACCACCCCCTACATGACT
ATCAAAGGATCAGTGATTGCCAACTGCAAGATGACCACATGCCGCTGCGTGAATCCTCCAGGTATCATCAGCCAGAA
CTATGGAGAAGCTGTGTCCCTAATTGATAAGCAGTCATGCAATGTGTTAAGCCTGGGCGGCATCACCTTACGGCTGA
GTGGAGAATTTGATGTCACTTATCAGAAGAACATCAGCATACAGGACAGCCAGGTCATCATAACAGGCAATCTGGAC
ATCTCAACAGAGCTTGGAAATGTCAACAACTCGATCAGCAATGCTTTGAATAAGCTGGAGGAAAGCAACAGGAAGCT
AGACAAGGTGAATGTGAAACTGACCAGCACTTCTGCTCTGATCACCTACATTGTGTTGACTATCATATCTCTTGTGT
TTGGCATCCTGAGCCTCATCCTGGCCTGCTACCTGATGTACAAGCAAAAAGCGCAACAAAAGACACTGTTATGGCTG
GGGAACAACACACTGGACCAGATGAGAGCCACCACCAAGATGTGA
(SEQ ID NO: 13, fully optimized version of SEQ ID NO: 12 ORF obtained from
NCBI reference sequence number DQ195265. 1 which is incorporated by
reference in its entirety.)

### Example 9: Insertion of the Codon Pair Optimized Fusion Gene into the ILTV Genome

CRISPR/Cas9 editing technologies will be used to generate rILTV/F. Briefly, LMH cells will be transfected with Ribonucleoprotein Complexes (RNPs) Cas9/Cpf1 with complexed guide as well as repair templates containing a promoter, NDV fusion gene, and selection cassette. The guides will be directed to cut at PAM sites in the targeted insertion sites. The selection cassette carries a green florescent protein (GFP) gene. These components will be introduced by nucleofection with a LT LMH adapted strain and selected based on fluorescing plaques. Once a candidate is identified, the selection cassette will be removed using CRISPR/Cas targeted to the two sgA PAM sequences positioned before and after the selection cassette.

### Example 10: Evaluation of In Vitro Codon Pair Optimized Fusion Gene Expression and Genetic Stability of rILTV/F

Expression levels of the fusion gene in rILTV/F candidates with different insertion sites will be evaluated by in vitro methods and those with highest relative expression will be selected. To determine the genetic stability, the insert site will be sequenced to verify the inserted DNA before and after 10 passages in LHM cells. Stable rILTV/F candidates will be selected for in vivo ND efficacy.

### Example 11: ND Efficacy to Evaluate Insertion Sites of rILTV/F Candidates

To determine the expression of the fusion gene from various insertion sites in the LT genome. Briefly, 3-week-old SPF chickens will be vaccinated by eye-drop, held for 20 days for immunity to develop and observed daily for post vaccination reactions. At the end of the 20-day period, blood and oropharyngeal swabs will be collected for serology and detection of vaccine virus, respectively and then chickens will be challenged with a neurotrophic NDV by the intramuscular route. Chickens will be observed daily for 14 days for neurological signs such as head or muscle tremors, torticollis, paralysis of one wing or one leg and mortality (Miller, 2013). Successful outcomes will be determined by comparing the efficacy of the various rILTV/F candidates with the fusion gene inserted into different insertion sites.

TABLE 1
Study design for ND efficacy to determine
the best insertion site into the LT genome.

| Group | Group | Vaccine | Vaccination | | | |
|---|---|---|---|---|---|---|
| # | purpose | name | age | dose (10^{x}) | route | # chickens |
| 1 | controls | Placebo-vaccinated, non- | 3 | placebo | eye | 5 |
| | | challenged negative controls | week | | drop | |
| 2 | | Placebo-vaccinated, | | | | 10 |
| | | challenged positive controls | | | | |
| 3 | test | rILTV/F¹ candidate 1 | | 2.0-3.0 | | 10 |
| 4 | | rILTV/F candidate 2 | | TCID₅₀² | | 10 |
| 5 | | rILTV/F candidate 3 | | | | 10 |

rILTV/F¹ = F gene of Newcastle disease virus inserted into the laryngotracheitis virus genome
TCID₅₀² = tissue culture infectious dose in 50%

### Example 12: ND Efficacy in Maternal Antibody Positive Chicks

To verify that maternal antibodies specific to ND do not interfere with day-old vaccination, efficacy will be evaluated by vaccinating layer or broiler chickens at day-old. Briefly, to prove that maternal antibodies to NDV are present, several day-old chickens will be bled and serology conducted. Other day-old chickens will be vaccinated by eye-drop, and held for 6 to 7 weeks or until the maternal antibody decreases. At the end of the 6 to 7-week period, oropharyngeal swabs will be collected for detection of vaccine virus, and then chickens will be challenged with a neurotrophic NDV by the intramuscular route. Chickens will be observed daily for 14 days for neurological signs of ND as described above. Successful outcomes will be determined by comparing the results of rILTV/F candidates with a rHVT/ND licensed vaccine.

TABLE 2
Study design for ND efficacy in maternal antibody positive chickens.

| Group | Group | Vaccine | Vaccination | | | |
|---|---|---|---|---|---|---|
| # | purpose | name | age | dose (10^{x}) | route | # chickens |
| 1 | controls | Placebo-vaccinated, non- | Day | placebo | eye | 10 |
| | | challenged negative controls | old | | drop | |
| 2 | | Placebo-vaccinated, | | | | 20 |
| | | challenged positive controls | | | | |
| 3 | | rHVT/ND¹ | | 3000 pfu³ | | 20 |
| 4 | test | rILTV/F² candidate 1 | | 2.0-3.0 | | 20 |
| 5 | | rILTV/F candidate 2 | | TCID₅₀⁴ | | 20 |

rHVT/ND¹ = F gene of Newcastle disease virus inserted into the herpesvirus of turkey genome
rILTV/F² = F gene of Newcastle disease virus inserted into the laryngotracheitis virus genome
pfu³ = plaque forming units
TCID₅₀⁴ = tissue culture infectious dose in 50%

### Example 13: Generation of Deoptimized ILTV Virus Clones with Deoptimized Codon-Pair Sequences

CRISPR/Cas9 editing technologies will be used to generate deoptimized ILTV sequences. LMH cells will be used for LT replication in culture as well as introduction of deoptimized sequences. Briefly, LMH cells transfected with RNPs Cas9/Cpf1 with complexed guide as well as repair templates for each of the defined sequences targeted for deoptimization. Other methods to edit the ILTV genome may be used if CRISPR has limitations. Deoptimized target ORFs in deoptimized ILTV will be clonally isolated and verified by sequence analysis.

### Example 14: Evaluation of Genetic Stability and Replication Profiles of the Recombinant Viruses

LMH cells will be infected with purified virus stocks and evaluated for in vitro replication by analyzing virus titers at different time points. Virus stocks will also be assessed for genetic stability by sequencing for up to 20 continuous passages. If attenuation is not discernible using replication in vitro then, candidates will be screened for attenuation in vivo. Multiple candidates will be inoculated by the infraorbital route at equivalent doses to determine attenuation. Broilers will be observed daily for 10 days for clinical signs of LT as described above. In addition, chickens will be scored from 0 to 3 for nasal exudate between 3 and 7-days post challenge. On the days 3, 5, 7, and 9 post challenge, oropharyngeal swabs will be collected to evaluate viral load. At 10 days post challenge, weights will be obtained and tracheas collected for histopathology. Attenuated vaccine candidates are defined by 1) clinical signs and/or nasal exudate comparable to the tissue culture origin (TCO) vaccine 2) clinical signs and/or nasal exudate less than the parent/reference strain and 3) a significant difference in weights compared to the parent strain but not the TCO vaccine. Depending on the number of candidates screened, multiple studies may be performed. If candidates from different studies are selected, attenuation will be re-evaluated in the same study.

TABLE 3
Study design for LT screening by in vivo attenuation.

| Group | Group | Vaccine | Vaccination | | | |
|---|---|---|---|---|---|---|
| # | purpose | name | age | dose (10^{x}) | route | # chickens |
| 1 | controls | Parent | 3 | 3.0-4.0 | infraorbital | 10 |
| 2 | | TCO¹ | week | TCID₅₀³ | | 10 |
| 3 | test | deoptimized rILTV/F² | | | | 10 |
| | groups | candidate 1 | | | | |
| 4 | | deoptimized rILTV/F | | | | 10 |
| | | candidate 2 | | | | |
| 5 | | deoptimized rILTV/F | | | | 10 |
| | | candidate 3 | | | | |
| 6 | | deoptimized rILTV/F² | | | | 10 |
| | | candidate 4 | | | | |
| 7 | | deoptimized rILTV/F | | | | 10 |
| | | candidate 5 | | | | |
| 8 | | deoptimized rILTV/F | | | | 10 |
| | | candidate 6 | | | | |

TCO¹ = tissue culture origin vaccine
Deoptimized rILTV/F² = F gene of Newcastle disease virus inserted into the codon pair deoptimized laryngotracheitis virus genome
TCID₅₀³ = tissue culture infectious dose in 50%

### Example 15: LT Preliminary Efficacy and Safety in 3-Week-Old SPF Chickens

To determine the preliminary efficacy and safety of live attenuated LT candidates, the first efficacy study is designed with the inclusion of a safety test in the chickens from same hatch as the efficacy test. If this study is successful, the next step is to evaluate efficacy and safety in day-old SPF chickens and day-old broilers, optimal target age for administration. Efficacy. Briefly, 3-week-old SPF chickens will be vaccinated by eye-drop, the gold standard for administration, and the administered highest relevant dose estimated at 10³ to 10⁴ TCID₅₀ (tissue culture infectious dose in 50%) to give the vaccine the best chance to succeed. Then chickens will be held for 14 days for immunity to develop and observed daily for post vaccination reactions. At the end of the 14-day period, blood and oropharyngeal swabs will be collected for serology and detection of vaccine virus, respectively and then chickens will be challenged by the infraorbital route. Chickens will be observed daily for 10 days for clinical signs of LT as described above. In addition, chickens will be scored 0 to 3 for nasal exudate between 3 and 7-days post challenge. On the days 3, 5, 7, and 9 post challenge, oropharyngeal swabs will be collected to evaluate challenge virus load and on day 10 post challenge, tracheas will be collected for histopathology. For a successful vaccine outcome, at least 80% of the chickens in the positive control group must develop clinical signs of LT while 90% of the chickens in a vaccine group are free from LT clinical signs. In addition, the vaccine candidate should be similar to a control chicken embryo origin (CEO) vaccine by pre-challenge serology and post-challenge by the following, (1) a quantitative scoring method of nasal exudate from 0 to 3, (2) reduction of challenge virus load in the trachea and (3) microscopic tracheal lesions. Safety. Briefly, 3-week-old SPF chickens will be inoculated intratracheally with the vaccine candidate at the same dose used in the efficacy portion of the trial. Chickens will be observed daily for 14 days for mortality. Unsafe vaccine outcomes are defined as, if more than 20 percent of the chickens die during the observation period, the virus is unsatisfactory.

TABLE 4
Study design for preliminary LT efficacy and safety in 3-week-old SPF chickens.

| Group | Group | Vaccine | Vaccination | | | |
|---|---|---|---|---|---|---|
| # | purpose | name | age | dose (10^{x}) | route | # chickens |
| 1 | efficacy | Placebo-vaccinated, | 3 | placebo | eye | 10 |
| | controls | non-challenged | week | | drop | |
| | | negative controls | | | | |
| 2 | | Placebo-vaccinated, | | | | 20 |
| | | challenged positive | | | | |
| | | controls | | | | |
| 3 | | CEO¹ | | 3.0 EID₅₀³ | | 20 |
| 4 | efficacy | deoptimized rILTV/F² | | 3.0-4.0 | | 20 |
| | test | candidate 1 | | TCID₅₀⁴ | | |
| 5 | groups | deoptimized rILTV/F | | | | 20 |
| | | candidate 2 | | | | |
| 6 | safety controls | CEO | 3 | 3.0 EID₅₀ | intratracheal | 25 |
| 7 | safety test | deoptimized rILTV/F | week | 3.0-4.0 | | 25 |
| | groups | candidate 1 | | TCID₅₀ | | |
| 8 | | deoptimized rILTV/F | | | | 25 |
| | | candidate 2 | | | | |

CEO¹ = embryo origin vaccine
Deoptimized rILTV/F² = F gene of Newcastle disease virus inserted into the codon pair deoptimized laryngotracheitis virus genome
EID₅₀³ = embryo infectious dose in 50%
TCID₅₀⁴ = tissue culture infectious dose in 50%

### Example 16: LT Efficacy and Safety in Day-Old SPF Chickens

Optimally, administration is to day-old chickens by spray at the hatchery verses 3-week-old chickens vaccinated in the field by drinking water. Efficacy will be conducted as above except that the vaccine candidates will be administered by multiple, lowering doses to day-old SPF chickens. If efficacy is demonstrated in day-old SPF chickens, then safety and efficacy in broilers will be evaluated as outlined below. If efficacy is not demonstrated, then studies will be conducted in 3-week-old SPF and broiler chickens to verify efficacy and safety by drinking water administration.

TABLE 5
Study design for LT efficacy and safety in day-old SPF chickens.

| Group | Group | Vaccine | Vaccination | | | |
|---|---|---|---|---|---|---|
| # | purpose | name | age | dose (10^{x}) | route | # chickens |
| 1 | efficacy | Placebo-vaccinated, | Day | placebo | eye | 10 |
| | controls | non-challenged | old | | drop | |
| | | negative controls | | | | |
| 2 | | Placebo-vaccinated, | | | | 40 |
| | | challenged positive | | | | |
| | | controls | | | | |
| 3 | | CEO¹ | | 3.0 EID₅₀³ | | 40 |
| 4 | efficacy | deoptimized rILTV/F² | | 3.0-4.0 | | 40 |
| | test | candidate 1 | | TCID₅₀⁴ | | |
| 5 | groups | Candidate 2 or | | | | 40 |
| | | candidate 1 at lower | | | | |
| | | dose | | | | |
| 6 | safety controls | CEO | Day | 3.0 EID₅₀ | intratracheal | 25 |
| 7 | safety test | deoptimized rILTV/F | old | 3.0-4.0 | | 25 |
| | groups | candidate 1 | | TCID₅₀ | | |
| 8 | | Candidate 2 or | | | | 25 |
| | | candidate 1 at lower | | | | |
| | | dose | | | | |

CEO¹ = embryo origin vaccine
Deoptimized rILTV/F² = F gene of Newcastle disease virus inserted into the codon pair deoptimized laryngotracheitis virus genome
EID₅₀³ = embryo infectious dose in 50%
TCID₅₀⁴ = tissue culture infectious dose in 50%

### Example 17: LT Safety in Day-Old Broilers

For a thorough proof of concept, we will also evaluate safety in broilers since more post vaccination reactions are observed with the current CEO vaccines in broilers than in layers. In addition, post vaccination reactions will be evaluated for vaccine candidates in combination with other normal hatchery vaccinations such as infectious bronchitis (IB) vaccines. Briefly, day-old broilers will be vaccinated by eye drop with either vaccine candidates or vaccine candidates in combination with IB vaccine. Comparative groups will be vaccinated with a CEO vaccine or CEO vaccine and IB vaccine. Respiratory responses will be evaluated daily for 14 days for clinical signs LT and IB including gasping, coughing, sneezing, tracheal rales, and nasal discharge (Jackwood, 2013). On the days 3, 5, 7, and 9 post inoculation, oropharyngeal swabs will be collected to evaluate LT and IB load. At 10 days post inoculation, weights will be obtained and tracheas collected for histopathology. Unsafe outcomes are defined by 1) dramatically exacerbated respiratory reactions and/or increased LT vaccine load when in combination with IB compared to groups without IB and 2) significant weight loss in groups with IB compared to groups without IB.

TABLE 6
Study design for LT safety in day-old broilers.

| Group | Group | Vaccine | Vaccination | | | |
|---|---|---|---|---|---|---|
| # | purpose | name | age | dose (10^{x}) | route | # chickens |
| 1 | control | CEO¹ | Day | 3.0 EID₅₀³ | eye | 25 |
| 2 | test groups | deoptimized rILTV/F² | old | 3.0-4.0 | drop | 25 |
| | | candidate 1 | | TCID₅₀⁴ | | |
| 3 | | Candidate 2 or candidate 1 | | | | 25 |
| | | at lower dose | | | | |
| 4 | control | CEO + IB⁵ vaccine | | 3.0 TCID₅₀ + | | 25 |
| 5 | test groups | deoptimized rILTV/F | | label | | 25 |
| | | candidate 1 + IB vaccine | | | | |
| 6 | | Candidate 2 or candidate 1 | | | | 25 |
| | | at lower dose + IB vaccine | | | | |

CEO¹ = embryo origin vaccine
Deoptimized rILTV/F² = F gene of Newcastle disease virus inserted into the codon pair deoptimized laryngotracheitis virus genome
EID₅₀³ = embryo infectious dose in 50%
TCID₅₀⁴ = tissue culture infectious dose in 50%
IB⁵ = infectious bronchitis

### Example 18: LT Efficacy in Day-Old Broilers

To verify LT efficacy in a day-old broiler, the study will be conducted as above for 3-week-old, SPF chickens except the vaccine candidates will be administered by spray to day-old broilers.

TABLE 7
Study design for LT efficacy in day-old broilers.

| Group | Group | Vaccine | Vaccination | | | |
|---|---|---|---|---|---|---|
| # | purpose | name | age | dose (10^{x}) | route | # chickens |
| 1 | controls | Placebo-vaccinated, non- | Day | placebo | spray | 10 |
| | | challenged negative | old | | | |
| | | controls | | | | |
| 2 | | Placebo-vaccinated, | | | | 40 |
| | | challenged positive | | | | |
| | | controls | | | | |
| 3 | | CEO¹ | | 3.0 EID₅₀³ | | 40 |
| 4 | test groups | deoptimized rILTV/F² | | 3.0-4.0 | | 40 |
| | | candidate 1 | | TCID₅₀⁴ | | |
| 5 | | Candidate 2 or candidate 1 | | | | 40 |
| | | at lower dose | | | | |

CEO¹ = embryo origin vaccine
Deoptimized rILTV/F² = F gene of Newcastle disease virus inserted into the codon pair deoptimized laryngotracheitis virus genome
EID₅₀³ = embryo infectious dose in 50%
TCID₅₀⁴ = tissue culture infectious dose in 50%

### Example 19: LT Efficacy in Maternal Antibody Positive Chicks and Minimum Protective Dose

To verify that maternal antibodies specific to LT do not interfere with day-old vaccination, efficacy will be evaluated by vaccinating layer or broiler chickens at day-old. In addition, the minimum protective dose will be determined. Efficacy will be conducted as above except that the vaccine candidates will be administered by multiple, lowering doses to day-old maternal antibody positive chickens instead of day-old SPF chickens. One outcome is that the lowest dose is expected to fail efficacy, verifying the endpoint of the dose response.

TABLE 8
Study design for LT efficacy in maternal antibody
positive chicks and minimum protective dose.

| Group | Group | Vaccine | Vaccination | | | |
|---|---|---|---|---|---|---|
| # | purpose | name | age | dose (10^{x}) | route | # chickens |
| 1 | controls | Placebo-vaccinated, non- | Day | placebo | spray | 10 |
| | | challenged negative controls | old | | | |
| 2 | | Placebo-vaccinated, | | | | 40 |
| | | challenged positive controls | | | | |
| 3 | | CEO¹ | | 3.0 EID₅₀³ | | 40 |
| 4 | test | deoptimized rILTV/F² | | dose 1 | | 40 |
| | groups | candidate 1 | | | | |
| 5 | | deoptimized rILTV/F | | dose 2 | | 40 |
| | | candidate 1 | | | | |
| 6 | | deoptimized rILTV/F | | dose 3 | | 40 |
| | | candidate 1 | | | | |

CEO¹ = embryo origin vaccine
Deoptimized rILTV/F² = F gene of Newcastle disease virus inserted into the codon pair deoptimized laryngotracheitis virus genome
EID₅₀³ = embryo infectious dose in 50%

### Example 20

Summary statistics were derived in Excel from outputs of the deoptimization method disclosed herein using all ILTV CDS sequences. This excluded sequences longer than 3000 nucleotides due to limitations of synthesis by integrated DNA technology (IDT) techniques.

Table 9 below provides the summary statistics. A trimmed sequence is the sequence between the start and stop codons (the ORF/open reading frame). Deoptimized sequences include sequences following deoptimization methods disclosed herein. Repaired sequences include sequences after both deoptimization and repair according to methods disclosed herein. Scores and sequence scores are codon pair bias (CPB) scores. The IDT score is the complexity value provided by IDT's tools.

TABLE 9

| | | | | | Trimmed | | | |
|---|---|---|---|---|---|---|---|---|
| | | | Percent | Percent | Integrated | | | |
| | | | Difference | Difference | DNA | | | |
| | | | Deoptimized | Repaired | Technologies | Deoptimized | Repaired | Repaired |
| | Deoptimized | Trimmed | to | to | (IDT) | IDT | IDT | Sequence |
| | Score | Score | Trimmed | Trimmed | Score | Score | Score | Score |
| Min | −0.87 | −0.19 | 57.26 | 33.20 | 0.00 | 0.00 | 0.00 | −0.79 |
| Max | −0.73 | 0.01 | 72.73 | 66.25 | 64.10 | 65.00 | 30.00 | −0.44 |
| Mean | −0.79 | −0.09 | 66.00 | 51.55 | 2.88 | 14.94 | 1.25 | −0.61 |
| Median | −0.79 | −0.09 | 66.29 | 51.66 | 0.00 | 11.10 | 0.00 | −0.61 |

Table 9 displays that a 14.5% reduction in the difference between deoptimized and trimmed sequences was observed following the use of repair methods. Additionally, a 23% reduction in deoptimization score was observed following the use of repair methods. A 57% reduction in IDT synthesis complexity was also observed when trimmed sequences underwent repair methods.

### Example 21

Table 10 below shows the number of sequences (based on the same ILTV genes as Table 9) that were too complex for IDT to synthesize. From top to bottom is each stage of the algorithm. That is, there were 4 trimmed sequences, 41 deoptimized sequences, and 3 deoptimized and repaired sequences that were too complex for synthesis by IDT techniques.

TABLE 10

| Stage | Too Complex |
|---|---|
| Trimmed | 4 |
| Deoptimized | 41 |
| Repaired | 3 |

### Example 22: Forward and Backward Repair

Forward and backward repairs were performed according to the present disclosure. As an example, parts of a sequence A were repaired using codons from B resulting in sequence C.

A first round of repair occurred where a deoptimized sequence was reverted to an original sequence (WIP sequence 1). A second round occurred where the original sequence (WIP sequence 1) was then deoptimized according to methods of the disclosure (WIP sequence 2). A third round then occurred where the deoptimized sequence (WIP sequence 2) was reverted to an original sequence (Repaired sequence).

### Example 23: Codon Pair Optimization of Fibroblast Activation Protein Gene

Genes for the human, chicken, and consensus FAP proteins listed below will be codon pair optimized to the bias of the genome of Feline cats. Increased expression of these proteins will be tested by ELISA when CRFK feline cells are transfected with the sequences below in expression plasmids. There will be a higher expression of the codon pair optimized protein in this cat cell line than one or more of the original sequence for human, chicken, and consensus sequences.

### Human FAP:

(SEQ ID NO: 14, human FAP.)
ATGGATTGGACCTGGATCCTGTTCCTGGTGGCTGCCGCTACAAGGGTGC
ACTCCCTCAGGCCCTCCAGGGTCCACAACTCCGAAGAGAATACTATGAG
GGCCCTCACACTGAAGGATATCCTGAACGGCACATTCTCCTATAAAACC
TTCTTTCCAAATTGGATCAGCGGCCAAGAGTACCTCCACCAGAGCGCCG
ACAATAACATCGTGCTGTACAATATCGAGACCGGACAAAGCTATACAAT
CCTCTCCAACAGAACCATGAAATCCGTCAATGCCTCCAATTATGGACTC
TCCCCAGACAGACAATTCGTCTACCTGGAGTCCGACTACTCCAAGCTCT
GGAGATATAGCTATACCGCCACATATTACATTTACGACCTCAGCAACGG
CGAATTCGTGAGGGGGAACGAGCTCCCCAGGCCCATCCAGTACCTGTGT
TGGAGCCCCGTGGGCAGCAAGCTCGCCTACGTCTACCAGAACAATATCT
ATCTCAAGCAAAGGCCCGGCGACCCTCCCTTCCAGATTACCTTTAATGG
CAGAGAGAACAAGATCTTTAACGGGATCCCTGATTGGGTCTATGAAGAG
GAAATGCTGGCCACTAAGTACGCCCTGTGGTGGAGCCCCAACGGCAAGT
TCCTGGCCTACGCTGAGTTTAACGATACAGACATTCCAGTGATCGCTTA
TAGCTACTATGGCGATGAGCAGTATCCCAGAACCATCAACATCCCCTAC
CCCAAGGCCGGCGCCAAGAATCCAGTCGTGAGAATTTTCATCATTGACA
CAACCTACCCCGCCTACGTCGGCCCCCAGGAAGTCCCTGTGCCTGCTAT
GATCGCCAGCTCCGACTACTATTTCAGCTGGCTCACTTGGGTCACCGAT
GAGAGAGTGTGCCTCCAGTGGCTCAAGAGGGTCCAGAACGTCTCCGTGC
TCAGCATTTGCGATTTCAGAGAAGACTGGCAGACTTGGGATTGTCCCAA
AACCCAGGAACACATCGAGGAATCCAGGACCGGCTGGGCTGGAGGGTTT
TTCGTCAGCACCCCAGTCTTCAGCTACGATGCTATCAGCTACTATAAAA
TCTTTTCCGATAAAGACGGCTACAAACATATCCACTATATCAAGGATAC
AGTCGAGAACGCCATCCAGATCACATCCGGCAAATGGGAGGCCATTAAT
ATTTTCAGAGTGACACAGGACAGCCTGTTTTACAGCTCCAACGAGTTCG
AAGAGTATCCTGGCAGGAGAAACATTTATAGAATCAGCATCGGAAGCTA
TCCACCCTCCAAGAAATGCGTCACATGCCACCTGAGAAAAGAGAGATGC
CAGTATTACACTGCTAGCTTCAGCGATTATGCCAAGTATTACGCTCTGG
TGTGTTATGGGCCTGGAATTCCCATTAGCACTCTGCACGATGGCAGAAC
TGACCAGGAAATTAAAATCCTGGAGGAAAACAAGGAACTGGAAAACGCC
CTGAAGAACATCCAGCTGCCCAAAGAAGAGATCAAGAAACTCGAGGTGG
ACGAGATTACTCTGTGGTACAAGATGATTCTCCCACCCCAATTTGACAG
AAGCAAGAAATATCCCCTCCTGATCCAGGTCTACGGCGGGCCCTGTTCC
CAGAGCGTGAGGAGCGTGTTCGCCGTGAACTGGATTTCCTACCTGGCCT
CCAAGGAAGGAATGGTGATCGCCCTGGTGGACGGAAGAGGAACCGCCTT
CCAAGGGGATAAACTCCTGTACGCCGTGTATAGAAAACTGGGGGTGTAC
GAAGTCGAGGATCAGATCACTGCCGTGAGGAAGTTTATCGAAATGGGCT
TCATTGACGAGAAGAGGATCGCCATTTGGGGCTGGGCTTACGGAGGCTA
CGTGTCCAGCCTCGCTCTGGCTAGCGGCACCGGGCTCTTTAAGTGCGGG
ATCGCCGTGGCCCCCGTCTCCAGCTGGGAGTATTACGCTAGCGTGTACA
CAGAAAGATTCATGGGCCTGCCAACAAAAGATGACAATCTGGAACACTA
TAAGAATAGCACTGTCATGGCTAGGGCTGAGTACTTTAGGAACGTCGAT
TATCTCCTGATTCACGGCACTGCTGACGATAACGTGCACTTCCAGAACT
CCGCCCAGATTGCTAAAGCTCTCGTGAACGCTCAGGTGGACTTCCAGGC
TATGTGGTACTCCGACCAAAATCATGGGCTCAGCGGACTCAGCACAAAC
CACCTGTATACCCACATGACTCATTTTCTGAAACAATGTTTTTCCCTGA
GCGACTAAA

### Codon Pair Optimized Human FAP:

(SEQ ID NO: 15, codon pair optimized human FAP.)
ATGGACTGGACCTGGATCCTCTTCCTGGTGGCTGCTGCCACCCGGGTGC
ACAGCCTGCGGCCCAGCCGGGTGCACAACAGCGAGGAGAACACCATGAG
AGCCCTGACCCTGAAGGACATCCTGAATGGCACCTTCTCCTACAAGACC
TTCTTCCCCAACTGGATCAGCGGCCAGGAGTACCTGCACCAGAGCGCCG
ACAACAACATCGTGCTGTACAACATTGAAACAGGCCAGAGCTACACCAT
CCTGAGCAACAGGACCATGAAGTCTGTGAATGCCAGCAACTATGGCCTG
TCCCCAGACAGGCAGTTTGTGTACCTGGAGAGTGACTACAGCAAGCTGT
GGAGATACAGCTACACAGCCACCTACTACATCTATGACCTGAGCAATGG
AGAGTTTGTGAGAGGAAATGAGCTGCCCCGGCCCATCCAGTACCTGTGC
TGGAGCCCTGTGGGCAGCAAGCTGGCCTACGTGTACCAGAACAACATCT
ACCTGAAGCAGAGGCCTGGGGACCCCCCCTTCCAGATCACCTTCAATGG
CCGGGAGAACAAGATCTTCAATGGCATCCCAGACTGGGTGTATGAGGAG
GAGATGCTGGCCACCAAGTATGCCCTGTGGTGGAGCCCCAATGGCAAGT
TCCTGGCCTATGCCGAGTTCAATGACACAGACATCCCTGTGATTGCCTA
CAGCTACTATGGAGATGAGCAGTACCCCAGGACCATCAACATCCCCTAC
CCCAAGGCCGGGGCCAAGAACCCTGTGGTGCGCATCTTCATCATTGACA
CCACCTACCCAGCCTACGTGGGCCCCCAGGAGGTGCCTGTGCCTGCCAT
GATTGCCAGCAGTGACTACTACTTCTCCTGGCTGACCTGGGTGACAGAT
GAGCGGGTGTGCCTGCAGTGGCTGAAGAGGGTGCAGAATGTGTCTGTGC
TGAGCATCTGTGACTTCCGGGAGGACTGGCAGACCTGGGACTGCCCCAA
GACCCAGGAGCACATCGAGGAGAGCAGAACAGGCTGGGCCGGGGGCTTC
TTTGTGTCCACCCCTGTGTTCTCCTATGATGCCATCAGCTACTACAAGA
TCTTCAGTGACAAGGATGGCTACAAGCACATCCACTACATCAAGGACAC
CGTGGAGAATGCCATCCAGATCACCAGCGGCAAGTGGGAGGCCATCAAC
ATCTTCCGGGTGACCCAGGACAGCCTCTTCTACAGCAGCAATGAATTTG
AGGAGTACCCAGGAAGAAGAAACATCTACCGCATCAGCATTGGCAGCTA
CCCCCCCAGCAAGAAGTGTGTGACCTGCCACCTGAGGAAGGAGCGCTGC
CAGTACTACACAGCCTCCTTCAGTGACTATGCCAAGTACTATGCCCTGG
TGTGCTATGGCCCCGGCATCCCCATCAGCACCCTGCATGATGGAAGAAC
AGACCAGGAGATCAAGATCCTGGAGGAGAACAAGGAGCTGGAGAATGCC
CTGAAGAACATCCAGCTGCCCAAGGAGGAGATCAAGAAGCTGGAGGTGG
ATGAGATCACCCTGTGGTACAAGATGATCCTGCCCCCCCAGTTTGACCG
CAGCAAGAAGTACCCCCTGCTCATCCAGGTGTATGGAGGCCCCTGCAGC
CAGTCTGTGCGCAGCGTGTTTGCTGTGAACTGGATCAGCTACCTGGCCA
GCAAGGAGGGCATGGTGATTGCCCTGGTGGATGGCCGGGGCACAGCCTT
CCAGGGGGACAAGCTGCTGTATGCTGTGTACAGGAAGCTGGGCGTGTAT
GAGGTGGAGGACCAGATCACAGCTGTGAGGAAGTTCATCGAGATGGGCT
TCATTGATGAGAAGAGAATTGCCATCTGGGGCTGGGCCTATGGAGGCTA
CGTGTCCAGCCTGGCCCTGGCCAGCGGCACCGGCCTCTTCAAGTGTGGC
ATTGCTGTGGCCCCTGTGTCCTCCTGGGAGTACTATGCCTCTGTGTACA
CAGAGCGCTTCATGGGCCTGCCCACCAAGGATGACAACCTGGAGCACTA
CAAGAACAGCACCGTGATGGCCCGGGCCGAGTACTTCAGAAATGTGGAC
TACCTGCTCATCCACGGCACAGCAGATGACAACGTGCACTTCCAGAACA
GCGCCCAGATTGCCAAGGCCCTGGTGAATGCCCAGGTGGACTTCCAGGC
CATGTGGTACAGTGACCAGAACCACGGCCTGAGCGGCCTGAGCACCAAC
CACCTGTACACCCACATGACCCACTTCCTGAAGCAGTGCTTCAGCCTGA
GTGAC

### Chicken FAP:

(SEQ ID NO: 16, chicken FAP.)
ATGGATTGGACATGGATTCTGTTCCTGGTGGCCGCTGCCACCAGAGTGC
ACAGCCTCCTGCCCTCCAAAGTCGTGACAACTGTGGACGGCCCAAGGGC
TCTCACCCTCGATGACTATCTCAACGGAAACTTTCAGTACAAGACATAT
TTCCCCTATTGGGTCTCCGATTCCGAATACCTCCACCAGAACCAGGAAG
ATGACATTATCCTCTTCAATGTGGACATGAATTACCTCACTACCATCAT
GACCAACTCCACCATGAAGCAGGTGAACGCCAGCAATTACGTGATGAGC
TCCGACAAGTATTTTATCGCTCTGGAAAGCAATTATTCCAAGCTCTGGA
GATATAGCTACACCGCCAGCTATCACATTTATGATCTCATCTACGGAGG
CTTCGTGACCGAAAATCAGCTGCCCCACAAAATTCAGTACATTTCCTGG
AGCCCCGTCGGCCACAAGCTGGCTTACGTCTACCAGAACAATATCTACC
TCAAACAAAGCCCCAGAGAGGCCCCAATTAAACTCACCTCCGACGGCAA
AGAAAATGAAATCTTTAACGGAATCCCTGATTGGGTGTACGAAGAGGAA
ATGCTGGCTACCAAATACGCCCTCTGGTGGAGCCCAAGCGGGAAATACC
TCGCCTACGTGCAGTTTAACGACTCCGATATTCCAGTGATTGAGTATTC
CTACTTCGGAGAGGACCAGTACCCCAGAAAAATTATCATTCCATATCCT
AAGGCCGGAGCTAAGAACCCTACCGTGAAAGTGTTCATCGTCGACACTA
CAAACATCGAAGCCTTTGGCCCTAAAGAAGTCCCTGTGCCAGCCGTGAT
CGCTTCCAGCGACCACTATTTCACCTGGCTGACCTGGGTGACCGACTCC
AGAGTCGGCGTGCAATGGCTGAAGAGGATTCAGAACTTCTCCGTCCTGG
CCATCTGCGACTTCAAAGAAAACAGCAACACATGGGACTGTCCCGAAAA
CCAGCAACATATTGAAGAGTCCCAAACAGGCTGGGCTGGAGGGTTTTTC
GTCTCCGCCCCATATTTTACATCCGATGGCAGCTCCTATTACAAGATCT
TCAGCGACAAGAACGGGTATAAACACATCCACTACATCAATGGCAGCGT
CGAAAACGCCATCCAGATTACCAGCGGCGAATGGGAAGCCATTTACATT
TTCAGAGTGACCAACGATGCCATTTTCTATAGCTCCAACGAATTTGAGG
GGTATCCCGGCAGGAGAAACATCTACAAAATTAGCATCGGATCCAAGCC
AATTAGAAAGCTGTGCATCACTTGTAATCTCAGGAAGGAAAGGTGTCAG
TATTACACCGCCAGATTCAGCGAGAGGAGCAAGTATTACGCCCTGATCT
GCTACGGGCCTGGCATTCCTATCAGCACCCTGTTTGAGACAGAAAGCGA
CAGAGAACTCAGAATCCTGGAAGACAACCAGGAGCTCCAGTCCGCCCTG
CAAGAGATCATTCTGCCCAAGGAAGAGATTAACAAACTCGAGGTGGACG
GAATCACCCTGTGGTATAAGATGCTGATTCCCCCTCAATTCGATAGGAG
CAAAAAGTACCCACTGCTCATCCAGGTGTATGGGGGCCCATGCTCCCAG
AATGTCAAGCACACATTCAGCATCAGCTGGATTACATACCTGGCCAGCA
AAGAAGGGATTATCGTCGCCCTGGTGGACGGAAGAGGCACCGCCTACCA
GGGAGATAAGATCCTGCACGCCGTCTATAGAAGGCTCGGGGTGTACGAA
GTCGAGGACCAAATTTCCGCCGTGAAGAAATTCATTGAGATGGGCTTCA
TTGATGAAAAGAGAATCGCTATCTGGGGCTGGGCTTATGGGGGCTATGT
CACCTCCCTGGCCCTGGGCAGCGGGTCCGGCGTGTTCAAATGCGGCATC
GCTGTGGCCCCCGTGTCCAGCTGGGAGTACTATGCCAGCATCTATACTG
AAAGGTTTATGGGCCTCCCCGTGGAGAGCGATAACCTGGAACACTATAA
AAACAGCACAGTGATGGCTAGAGCCAAAAACTTCCAAAATGTGGAATAC
CTCCTGATCCACGGGACTGCCGATGACAACGTCCACTTCCAAAATAGCG
CCCAGATCGCCAAGGCCCTGGTCAACGCTCAGGTGGACTTTCAAGCCAT
GTGGTACACAGATCAGAACCACGGCATTCCCGGGCTCAGCTCCAAGCAT
CTCTATACCCACATGACTCACTTCCTGAAGCAGTGCTTCAGCCTGAGCG
AATAAA

### Codon Pair Optimized Chicken FAP:

(SEQ ID NO: 17, codon pair optimized chicken FAP.)
ATGGACTGGACCTGGATCCTCTTCCTGGTGGCTGCTGCCACCCGGGTGC
ACAGCCTGCTGCCCAGCAAGGTGGTGACCACCGTGGATGGCCCCCGGGC
CCTGACCCTGGATGACTACCTGAATGGCAACTTCCAGTACAAGACCTAC
TTCCCCTACTGGGTGTCTGACAGTGAGTACCTGCACCAGAACCAGGAGG
ATGACATCATCCTCTTCAACGTGGACATGAACTACCTGACCACCATCAT
GACCAACAGCACCATGAAGCAGGTGAATGCCAGCAACTACGTGATGAGC
AGTGACAAGTACTTCATCGCCCTGGAGAGCAACTACAGCAAGCTGTGGA
GATACAGCTACACAGCCAGCTACCACATCTATGACCTCATCTATGGAGG
CTTTGTGACAGAGAACCAGCTGCCCCACAAGATCCAGTACATCTCCTGG
AGCCCTGTGGGCCACAAGCTGGCCTACGTGTACCAGAACAACATCTACC
TGAAGCAGAGCCCCCGGGAGGCCCCCATCAAGCTGACCAGTGATGGCAA
GGAGAATGAGATCTTCAATGGCATCCCAGACTGGGTGTATGAGGAGGAG
ATGCTGGCCACCAAGTATGCCCTGTGGTGGAGCCCCAGCGGCAAGTACC
TGGCCTACGTGCAGTTCAATGACAGTGACATCCCTGTGATTGAGTACAG
CTACTTTGGAGAGGACCAGTACCCCAGGAAGATCATCATCCCCTACCCC
AAGGCCGGGGCCAAGAACCCCACCGTGAAGGTGTTCATCGTGGACACCA
CCAACATCGAGGCCTTTGGCCCCAAGGAGGTGCCTGTGCCTGCTGTGAT
TGCCAGCAGTGACCACTACTTCACCTGGCTGACCTGGGTGACAGACAGC
CGGGTGGGCGTGCAGTGGCTGAAGAGGATCCAGAACTTCTCTGTGCTGG
CCATCTGTGACTTCAAGGAGAACAGCAACACCTGGGACTGCCCAGAGAA
CCAGCAGCACATCGAGGAGAGCCAGACAGGCTGGGCCGGGGGCTTCTTT
GTGTCTGCCCCCTACTTCACCAGTGATGGCAGCAGCTACTACAAGATCT
TCAGTGACAAGAATGGCTACAAGCACATCCACTACATCAATGGCTCTGT
GGAGAATGCCATCCAGATCACCAGCGGGGAGTGGGAGGCCATCTACATC
TTCCGGGTGACCAATGATGCCATCTTCTACAGCAGCAATGAATTTGAGG
GCTACCCAGGAAGAAGAAACATCTACAAGATCAGCATTGGCAGCAAGCC
CATCCGGAAGCTGTGCATCACCTGCAACCTGAGGAAGGAGCGCTGCCAG
TACTACACAGCCCGCTTCTCTGAGAGGAGCAAGTACTATGCCCTGATCT
GCTATGGCCCCGGCATCCCCATCAGCACCCTGTTTGAGACAGAGAGTGA
CCGGGAGCTGCGCATCCTGGAGGACAACCAGGAGCTGCAGAGCGCCCTG
CAGGAGATCATCCTGCCCAAGGAGGAGATCAACAAGCTGGAGGTGGATG
GCATCACCCTGTGGTACAAGATGCTGATCCCCCCCCAGTTTGACCGCAG
CAAGAAGTACCCCCTGCTCATCCAGGTGTATGGAGGCCCCTGCAGCCAG
AATGTGAAGCACACCTTCAGCATCTCCTGGATCACCTACCTGGCCAGCA
AGGAGGGCATCATCGTGGCCCTGGTGGATGGCCGGGGCACAGCCTACCA
GGGGGACAAGATCCTGCACGCCGTGTACCGGCGGCTGGGCGTGTATGAG
GTGGAGGACCAGATCTCTGCTGTGAAGAAGTTCATCGAGATGGGCTTCA
TTGATGAGAAGAGAATTGCCATCTGGGGCTGGGCCTATGGAGGCTACGT
GACCAGCCTGGCCCTGGGCAGCGGCAGCGGCGTGTTCAAGTGTGGCATT
GCTGTGGCCCCTGTGTCCTCCTGGGAGTACTATGCCAGCATCTACACAG
AGCGCTTCATGGGCCTGCCTGTGGAGAGTGACAACCTGGAGCACTACAA
GAACAGCACCGTGATGGCCCGGGCCAAGAACTTCCAGAATGTGGAGTAC
CTGCTCATCCACGGCACAGCAGATGACAACGTGCACTTCCAGAACAGCG
CCCAGATTGCCAAGGCCCTGGTGAATGCCCAGGTGGACTTCCAGGCCAT
GTGGTACACAGACCAGAACCACGGCATCCCCGGCCTGAGCAGCAAGCAC
CTGTACACCCACATGACCCACTTCCTGAAGCAGTGCTTCAGCCTGTCTG
AG

### Consensus FAP:

(SEQ ID NO: 18, consensus FAP.)
ATGGATTGGACCTGGATTCTCTTTCTGGTCGCCGCTGCCACTAGAGTGC
ACAGCCTCAGGCCTAGCAGGGTGCACAACAGCGAAGGGAACACCACTAG
GGCCCTCACCCTGAAGGACATCCTGAATGGGACATTCAGCTACAAGACC
TTCTTTCCTAATTGGATCAGCGGCCAGGAGTATCTCCACCAATCCACCG
ACAATAACATCGTGCTCTACAATATTGAGACCGGGGAATCCTATACCAT
TCTCAGCAACTCCACAATGAAATCCGTGAACGCCAGCAATTACGGACTG
TCCCCCGACAGGCAATTCGCTTATCTGGAGTCCGACTACTCCAAGCTGT
GGAGGTATAGCTATACTGCCACTTACCACATTTATGATCTGTCCAATGG
CGAATTTGTGAGAGGGAACGAGCTGCCCAGACCAATCCAGTACCTGTGC
TGGAGCCCAGTGGGGAGCAAGCTCGCTTACGTCTACCAGAACAATATTT
ATCTGAAACAGAGACCCGAAGACCCACCCTTTCAGATCACCTATAATGG
CAGGGAGAACAAGATTTTTAACGGCATCCCCGACTGGGTGTACGAAGAG
GAAATGCTGGCCACCAAATACGCCCTCTGGTGGTCCCCTAACGGCAAAT
TCCTGGCTTATGCCGAGTTCAACGATACCGACATCCCTGTCATCGCCTA
TAGCTATTACGGAGACGAGCAGTACCCTAGAACCATCAATATTCCCTAC
CCAAAAGCCGGCGCCAAAAACCCCGTCGTGAGGATCTTTATTATCGACA
CCACATACCCAGAGCACGTGGGACCCAGGGAGGTCCCCGTGCCTGCTAT
GATTGCCTCCAGCGACTACTATTTTTCCTGGCTCACCTGGGTGACCGAC
GAAAGGGTGTGCCTGCAGTGGCTGAAAAGGATCCAGAACGTGTCCGTGC
TCAGCATCTGCGATTTCAGGGAGGATTGGCAGACCTGGGATTGTCCTAA
GACACAGGAACACATTGAAGAGAGCAGAACAGGCTGGGCCGGCGGATTC
TTTGTGTCCACCCCCGTGTTTTCCTATGACGCTATTAGCTATTACAAAA
TCTTCTCCGATAAGGATGGCTATAAACATATTCACTACATCAAGGACAC
TGTGGAGAATGCCATTCAGATTACTAGCGGCAAGTGGGAGGCCATTAAC
ATCTTCAGAGTCACCCAGGATAGCCTGTTCTACTCCAGCAACGAGTTTG
AGGGATACCCCGGCAGGAGAAACATCTACAGGATTAGCATCGGCAGCTA
TCCCCCATCCAAGAAATGCATCACATGTCATCTGAGGAAGGAGAGATGC
CAGTACTATACAGCCAGCTTCTCCGACTATGCTAAATATTACGCCCTGG
TGTGTTATGGCCCTGGCCTCCCAATCTCCACTCTGCATGACGGGAGAAC
TGACCAAGAGATCAAGATTCTGGAGGAAAACAAGGAGCTGGAAAACGCT
CTGAAGAATATCCAACTGCCCAAGGAAGAGATCAAGAAACTGGAAGTGG
ATGAAATTACCCTGTGGTACAAAATGATCCTGCCTCCCCAATTCGATAG
ATCCAAAAAGTACCCCCTCCTGATCCAAGTGTACGGGGGCCCTTGCAGC
CAGTCCGTGAGGTCCGTGTTTGCCGTCAACTGGATCTCCTACCTGGCCA
GCAAGGAAGGGATCGTGATTGCCCTGGTGGATGGCAGAGGCACAGCTTT
CCAGGGAGATAAACTCCTGTACGCCGTCTATAGAAAACTCGGCGTGTAC
GAGGTGGAGGACCAGATTACCGCCGTGAGGAAGTTTATCGAGATGGGCT
TCATCGATGAGAAAAGGATCGCCATTTGGGGGTGGGCTTACGGAGGGTA
CGTGTCCAGCCTCGCCCTGGCCAGCGGAACAGGGCTGTTCAAGTGCGGG
ATCGCCGTCGCCCCTGTGAGCTCCTGGGAATATTACGCCTCCATCTACA
CCGAAAGGTTCATGGGCCTGCCCACTAAGGATGACAATCTGGAGCACTA
CAAGAACTCCACTGTCATGGCCAGGGCTGAGTACTTCAGAAATGTGGAC
TACCTCCTGATCCATGGCACAGCTGACGATAACGTGCATTTTCAGAACA
GCGCCCAGATTGCCAAAGCCCTGGTGAATGCCCAGGTGGATTTCCAGGC
CATGTGGTATAGCGACCAGAACCACGGAATCAGCGGCCTCAGCACCAAA
CACCTGTATACCCATATGACCCACTTCCTGAAGCAGTGTTTTTCCCTGA
GCGATTAAA

### Codon Pair Optimized Consensus FAP:

(SEQ ID NO: 19, codon pair optimized consensus FAP.)
ATGGACTGGACCTGGATCCTCTTCCTGGTGGCTGCTGCCACCCGGGTGCA
CAGCCTGCGGCCCAGCCGGGTGCACAACAGCGAGGGCAACACCACCCGGG
CCCTGACCCTGAAGGACATCCTGAATGGCACCTTCTCCTACAAGACCTTC
TTCCCCAACTGGATCAGCGGCCAGGAGTACCTGCACCAGAGCACAGACAA
CAACATCGTGCTGTACAACATTGAAACAGGAGAGAGCTACACCATCCTGA
GCAACAGCACCATGAAGTCTGTGAATGCCAGCAACTATGGCCTGTCCCCA
GACAGGCAGTTTGCCTACCTGGAGAGTGACTACAGCAAGCTGTGGAGATA
CAGCTACACAGCCACCTACCACATCTATGACCTGAGCAATGGAGAGTTTG
TGAGAGGAAATGAGCTGCCCCGGCCCATCCAGTACCTGTGCTGGAGCCCT
GTGGGCAGCAAGCTGGCCTACGTGTACCAGAACAACATCTACCTGAAGCA
GAGGCCTGAGGACCCCCCCTTCCAGATCACCTACAATGGCCGGGAGAACA
AGATCTTCAATGGCATCCCAGACTGGGTGTATGAGGAGGAGATGCTGGCC
ACCAAGTATGCCCTGTGGTGGAGCCCCAATGGCAAGTTCCTGGCCTATGC
CGAGTTCAATGACACAGACATCCCTGTGATTGCCTACAGCTACTATGGAG
ATGAGCAGTACCCCAGGACCATCAACATCCCCTACCCCAAGGCCGGGGCC
AAGAACCCTGTGGTGCGCATCTTCATCATTGACACCACCTACCCAGAGCA
CGTGGGCCCCCGGGAGGTGCCTGTGCCTGCCATGATTGCCAGCAGTGACT
ACTACTTCTCCTGGCTGACCTGGGTGACAGATGAGCGGGTGTGCCTGCAG
TGGCTGAAGAGGATCCAGAATGTGTCTGTGCTGAGCATCTGTGACTTCCG
GGAGGACTGGCAGACCTGGGACTGCCCCAAGACCCAGGAGCACATCGAGG
AGAGCAGAACAGGCTGGGCCGGGGGCTTCTTTGTGTCCACCCCTGTGTTC
TCCTATGATGCCATCAGCTACTACAAGATCTTCAGTGACAAGGATGGCTA
CAAGCACATCCACTACATCAAGGACACCGTGGAGAATGCCATCCAGATCA
CCAGCGGCAAGTGGGAGGCCATCAACATCTTCCGGGTGACCCAGGACAGC
CTCTTCTACAGCAGCAATGAATTTGAGGGCTACCCAGGAAGAAGAAACAT
CTACCGCATCAGCATTGGCAGCTACCCCCCCAGCAAGAAGTGCATCACCT
GCCACCTGAGGAAGGAGCGCTGCCAGTACTACACAGCCTCCTTCAGTGAC
TATGCCAAGTACTATGCCCTGGTGTGCTATGGCCCCGGCCTGCCCATCAG
CACCCTGCATGATGGAAGAACAGACCAGGAGATCAAGATCCTGGAGGAGA
ACAAGGAGCTGGAGAATGCCCTGAAGAACATCCAGCTGCCCAAGGAGGAG
ATCAAGAAGCTGGAGGTGGATGAGATCACCCTGTGGTACAAGATGATCCT
GCCCCCCCAGTTTGACCGCAGCAAGAAGTACCCCCTGCTCATCCAGGTGT
ATGGAGGCCCCTGCAGCCAGTCTGTGCGCAGCGTGTTTGCTGTGAACTGG
ATCAGCTACCTGGCCAGCAAGGAGGGCATCGTGATTGCCCTGGTGGATGG
CCGGGGCACAGCCTTCCAGGGGGACAAGCTGCTGTATGCTGTGTACAGGA
AGCTGGGCGTGTATGAGGTGGAGGACCAGATCACAGCTGTGAGGAAGTTC
ATCGAGATGGGCTTCATTGATGAGAAGAGAATTGCCATCTGGGGCTGGGC
CTATGGAGGCTACGTGTCCAGCCTGGCCCTGGCCAGCGGCACCGGCCTCT
TCAAGTGTGGCATTGCTGTGGCCCCTGTGTCCTCCTGGGAGTACTATGCC
AGCATCTACACAGAGCGCTTCATGGGCCTGCCCACCAAGGATGACAACCT
GGAGCACTACAAGAACAGCACCGTGATGGCCCGGGCCGAGTACTTCAGAA
ATGTGGACTACCTGCTCATCCACGGCACAGCAGATGACAACGTGCACTTC
CAGAACAGCGCCCAGATTGCCAAGGCCCTGGTGAATGCCCAGGTGGACTT
CCAGGCCATGTGGTACAGTGACCAGAACCACGGCATCAGCGGCCTGAGCA
CCAAGCACCTGTACACCCACATGACCCACTTCCTGAAGCAGTGCTTCAGC
CTGAGTGAC

### Example 24

### Methods and Materials:

Open reading frames (ORFs) that encode Gaussia luciferase (gLUC) were generated with varying degrees of dicodon optimization or deoptimization (including 100%, 75%, 50% or 25%), using the VICOPA tool. Dicodon usage, in this instance, was based on the canine genome. The products were synthesized as gblocks using integrated DNA technologies (IDT) techniques and cloned into an expression vector under the control of a cytomegalovirus (CMV) promoter. Sequence confirmed constructs were transfected via lipophilic transfection reagent into MyoK9 (canine myoblast cell line), and supernatant was harvested 24 hours post transfection. gLUC activity was assessed using the Gaussia Luciferase Glow Assay Kit (Pierce) and luminescence detected on a plate-based luminometer.

### Results:

Results were normalized based on transfection efficiency from each construct using renilla luciferase. Methods were repeated with an 8-hour collection period to demonstrate a more tunable response. Sequence correct clones were identified for all 8 inducible constructs that had the same gLUC opt/depot sequences.

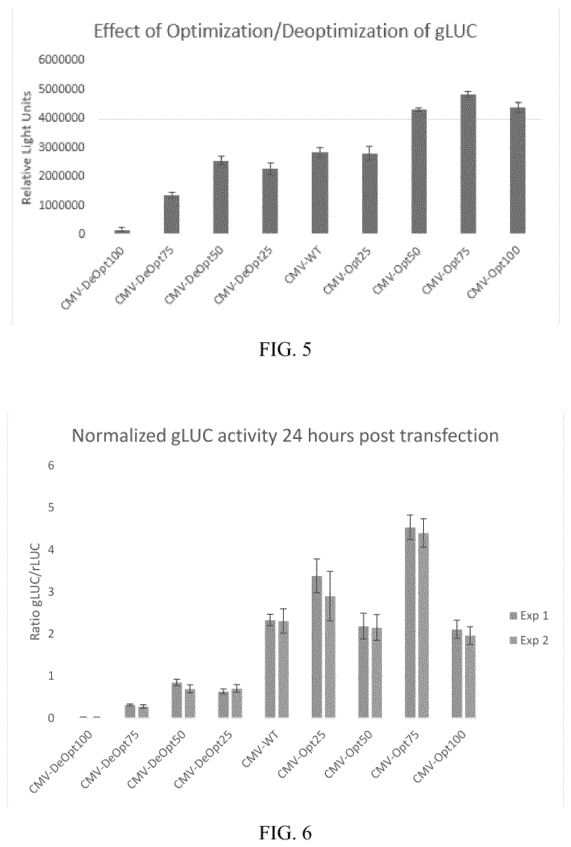

Luciferase values were imported into an excel spreadsheet. The average of luciferase activity from non-transfected cells were subtracted from values obtained the control and experimental transfectant, in order to subtract out background luciferase activity from the assay. Data displayed in FIG. 5 represents the average of four individual transfected wells for each construct.

### Example 25

Open reading frames (ORFs) encoding Gaussia luciferase (gLUC) were generated with varying degrees of dicodon optimization or deoptimization (including 100%, 75%, 50% or 25%), using the VICOPA tool. Dicodon usage was based on the canine genome. The products were synthesized as gblocks using integrated DNA technologies (IDT) and cloned into an expression vector under the control of a cytomegalovirus (CMV) promoter. Sequence confirmed constructs were transfected along with a constitutive rLUC construct at about 4% total DNA content (lipophilic transfection reagent) into MyoK9 (canine myoblast cell line), and supernatant was harvested 24 hours post transfection. Cell lysates gLUC activity was assessed using the Gaussia Luciferase Glow Assay Kit (Pierce) and luminescence detected on a plate-based luminometer. Luciferase values were imported into an excel spreadsheet.

FIG. 6 displays results including the average of four individual, transfected wells for each construct resulting from two independent experiments (Exp 1 and Exp 2). Results showed that use of the VICOPA tool could augment translational efficiency of an ORF.

### Example 26

Open reading frames (ORFs) encoding Gaussia luciferase (gLUC) were generated with varying degrees of dicodon optimization or deoptimization (including 100%, 75%, 50% or 25%), using the VICOPA tool. Dicodon usage was based on the canine genome. The products were synthesized as gblocks using integrated DNA technologies (IDT) and cloned into an expression vector under the control of a cytomegalovirus (CMV) promoter. Sequence confirmed constructs were transfected along with a constitutive rLUC construct at about 4% total DNA content (lipophilic transfection reagent) into MyoK9 (canine myoblast cell line). About 24 hours post-transfection, media was changed and supernatant was harvested about 6 hours later. gLUC activity was assessed using the Gaussia Luciferase Glow Assay Kit (Pierce) and luminescence detected on a plate-based luminometer. Luciferase values were imported into an excel spreadsheet.

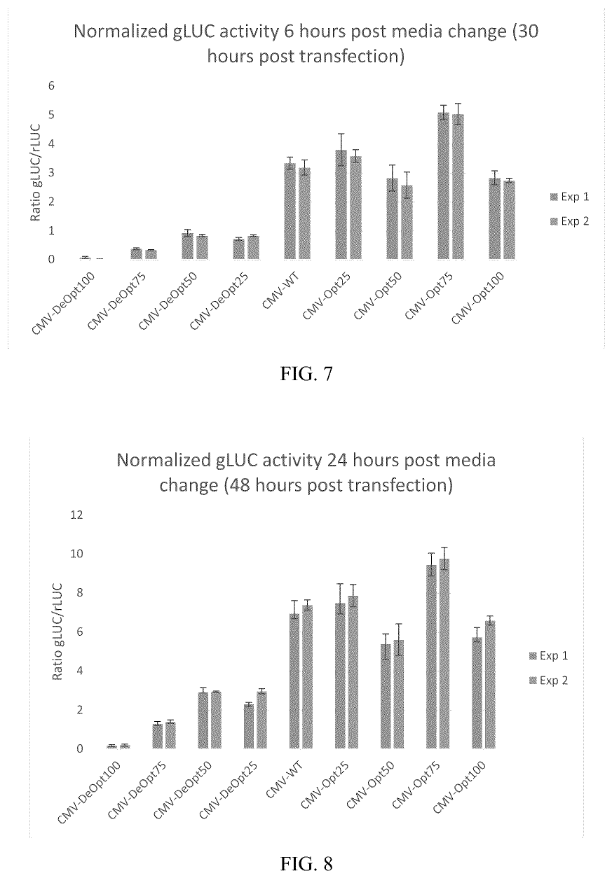

FIG. 7 displays results including the average of four individual, transfected wells for each construct resulting from two independent experiments (Exp 1 and Exp 2).

### Example 27

Open reading frames (ORFs) that encode Gaussia luciferase (gLUC) were generated with varying degrees of dicodon optimization or deoptimization (including 100%, 75%, 50% or 25%), using the VICOPA tool. Dicodon usage was based on the canine genome. The products were synthesized as gblocks integrated DNA technologies (IDT) and cloned into an expression vector under the control of a cytomegalovirus (CMV) promoter. Sequence confirmed constructs were transfected along with a constitutive rLUC construct at about 4% total DNA content (lipophilic transfection reagent) into MyoK9 (canine myoblast cell line). About 24 hours post-transfection, media was changed and supernatant was harvested about 24 hours later. gLUC activity was assessed using the Gaussia Luciferase Glow Assay Kit (Pierce) and luminescence detected on a plate-based luminometer. Luciferase values were imported into an excel spreadsheet.

FIG. 8 displays results including the average of four individual, transfected wells for each construct resulting from two independent experiments (Exp 1 and Exp 2).

### Example 28

Open reading frames (ORFs) that encode Gaussia luciferase (gLUC) were generated with varying degrees of dicodon optimization or deoptimization (including 100%, 75%, 50% or 25%), using the VICOPA tool. Dicodon usage was based on the canine genome. The products were synthesized as gblocks integrated DNA technologies (IDT) and cloned into an expression vector under the control of an inducible promoter. Sequence confirmed constructs were transfected along with a constitutive rLUC construct at about 4% total DNA content (lipophilic transfection reagent) into MyoK9 (canine myoblast cell line). About 24 hours post-transfection, media was changed with either ligand or vehicle-containing media, and supernatant was harvested about 6 hours later. gLUC activity was assessed using the Gaussia Luciferase Glow Assay Kit (Pierce) and luminescence detected on a plate-based luminometer. Luciferase values were imported into an excel spreadsheet.

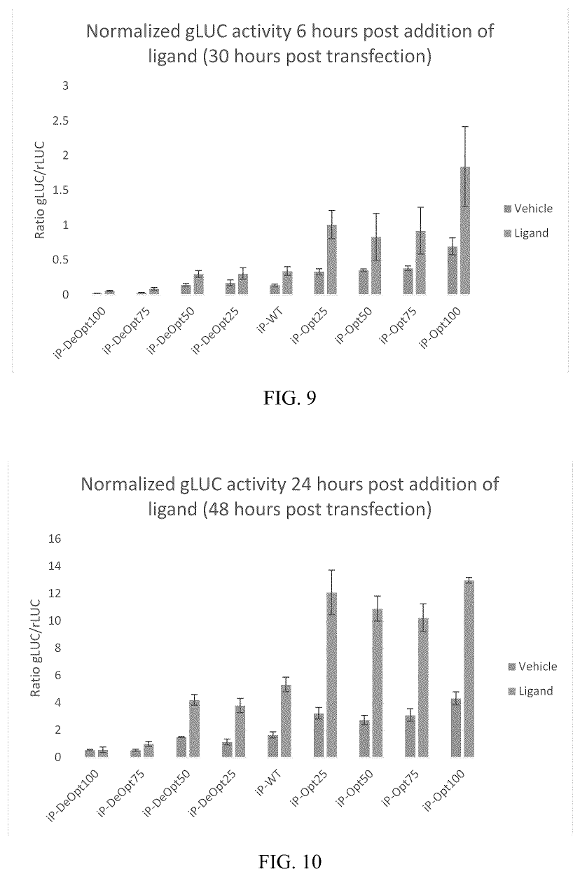

FIG. 9 displays results including the average of four individual, transfected wells for each construct resulting from two independent experiments (Exp 1 and Exp 2).

### Example 29

Open reading frames (ORFs) that encode Gaussia luciferase (gLUC) were generated with varying degrees of dicodon optimization or deoptimization (including 100%, 75%, 50% or 25%), using the VICOPA tool. Dicodon usage was based on the canine genome. The products were synthesized as gblocks integrated DNA technologies (IDT) and cloned into an expression vector under the control of a cytomegalovirus (CMV) promoter. Sequence confirmed constructs were transfected along with a constitutive rLUC construct at about 4% total DNA content (lipophilic transfection reagent) into MyoK9 (canine myoblast cell line). About 24 hours post-transfection, media was changed with either ligand or vehicle-containing media, and supernatant was harvested about 24 hours later. gLUC activity was assessed using the Gaussia Luciferase Glow Assay Kit (Pierce) and luminescence detected on a plate-based luminometer. Luciferase values were imported into an excel spreadsheet.

FIG. 10 displays results including the average of four individual, transfected wells for each construct resulting from two independent experiments (Exp 1 and Exp 2).

The data produced in Examples 25-28 above, as shown in FIGS. 6-10, resulted from transfections of the opt and depot gLUC constructs. Such data represented results using both constitutive and inducible promoter constructs for expression of sequences. Results using CMV constructs were surprising. A significant increase in gLUC was observed with CMV-opt75 and a lesser increase with CMV-opt25.

Deoptimization impacts were observed to be similar between CMV and inducible promoter constructs. A larger hit in gLUC activity was observed with the CMV constructs.

### Example 30

Gaussia luciferase sequences were optimized or deoptimized to selected levels defined by codon pair bias scores including 25, 50, 75 and 100. Optimized or deoptimized sequences, as well as wild-type sequences, were then compared by performing a standard nucleotide alignment of all sequences using a program called Geneious. The results are presented in Table 11 below which shows percent shared identity between the different optimized, deoptimized, or wild sequences.

The row and column titles of Table 11 (e.g., “Gaussia-Dura Luc OPT100”) indicate the codon pair bias optimization or deoptimization that was selected for the output sequence that delivered the sequences used. The x- and y-axes display the same sequences and identify which sequence pair is being compared. The numeric values on the interior of Table 11 display the percentage of identity shared between the two sequences being compared. As an example, the very top right cell displays a comparison of Gaussia-Dura Luc DEOPT100 and Gaussia-Dura Luc Wild Type where the two sequences share 79.6% of their sequence identities. The diagonal of Table 11 is blank because it represents identical sequences being compared.

TABLE 11

| | Gaussia- | Gaussia- | Gaussia- | Gaussia- | Gaussia- | Gaussia- | Gaussia- | Gaussia- | Gaussia- |
|---|---|---|---|---|---|---|---|---|---|
| | Dura Luc | Dura Luc | Dura Luc | Dura Luc | Dura Luc | Dura Luc | Dura Luc | Dura Luc | Dura Luc |
| | DEOPT100 | DEOPT75 | DEOPT50 | DEOPT25 | OPT25 | OPT50 | OPT75 | OPT100 | WT |
| Gaussia- | — | 93.4 | 89.0 | 85.6 | 78.8 | 76.4 | 75.1 | 74.3 | 79.6 |
| Dura Luc | | | | | | | | | |
| DEOPT100 | | | | | | | | | |
| Gaussia- | 93.4 | — | 94.0 | 89.6 | 82.5 | 80.7 | 79.9 | 77.5 | 83.3 |
| Dura Luc | | | | | | | | | |
| DEOPT75 | | | | | | | | | |
| Gaussia- | 89.0 | 94.0 | — | 95.3 | 87.9 | 86.2 | 84.0 | 81.9 | 88.7 |
| Dura Luc | | | | | | | | | |
| DEOPT50 | | | | | | | | | |
| Gaussia- | 85.6 | 89.6 | 95.3 | — | 92.6 | 89.2 | 86.9 | 85.1 | 93.4 |
| Dura Luc | | | | | | | | | |
| DEOPT25 | | | | | | | | | |
| Gaussia- | 78.8 | 82.5 | 87.9 | 92.6 | — | 96.6 | 94.3 | 92.4 | 97.1 |
| Dura Luc | | | | | | | | | |
| OPT25 | | | | | | | | | |
| Gaussia- | 76.4 | 80.7 | 86.2 | 89.2 | 96.6 | — | 97.4 | 94.8 | 93.7 |
| Dura Luc | | | | | | | | | |
| OPT50 | | | | | | | | | |
| Gaussia- | 75.1 | 79.9 | 84.0 | 86.9 | 94.3 | 97.4 | — | 96.8 | 91.4 |
| Dura Luc | | | | | | | | | |
| OPT75 | | | | | | | | | |
| Gaussia- | 74.3 | 77.5 | 81.9 | 85.1 | 92.4 | 94.8 | 96.8 | — | 90.1 |
| Dura Luc | | | | | | | | | |
| OPT100 | | | | | | | | | |
| Gaussia- | 79.6 | 83.3 | 88.7 | 93.4 | 97.1 | 93.7 | 91.4 | 90.1 | — |
| Dura Luc | | | | | | | | | |
| WT | | | | | | | | | |

That which is claimed is:

1. A vaccine composition for immunizing an avian subject against a pathogen comprising a recombinant Infectious Laryngotracheitis Virus (ILTV) vector, wherein the ILTV vector has been deoptimized.

2. The composition of claim 1, wherein the ILTV vector comprises a nucleic acid sequence of at least 95%, or 100% of SEQ ID NO:3.

3. The composition of claim 1, wherein the ILTV vector comprises a nucleic acid sequence of at least 95%, or 100% of SEQ ID NO:5.

4. The composition of claim 1, wherein the ILTV vector comprises a nucleic acid sequence of at least 95%, or 100% of SEQ ID NO:7.

5. The composition of claim 1, wherein the ILTV vector comprises a nucleic acid sequence of at least 95%, or 100% of SEQ ID NO:9.

6. The composition of claim 1, wherein the ILTV vector comprises a nucleic acid sequence of at least 95%, or 100% of SEQ ID NO:11.

7. A method for treating or preventing ILTV comprising administering to an avian in need thereof an effective amount of a recombinant ILTV vector.

8. The method of claim 7, wherein the recombinant ILTV vector comprises a nucleic acid sequence of at least 95%, or 100% of SEQ ID NO: 3, SEQ ID NO: 5, SEQ ID NO:7, SEQ ID NO: 9, or SEQ ID NO: 11.

9. The method of claim 7, wherein the effective amount of the recombinant ILTV vector is administered in ovo after at least two weeks since embryonation day.

10. The method of claim 7, wherein the effective amount of the recombinant ILTV vector comprises more than one dose administered at separate times.

11. A vaccine composition for immunizing an avian subject against a pathogen comprising:
a Herpes Virus of Turkeys (HVT) viral vector;
a fusion gene of Newcastle disease (ND) virus inserted into the recombinant HVT vector and
wherein the recombinant HVT vector having the fusion gene of Newcastle disease (ND) virus is administered to the subject in need thereof.

12. The composition of claim 11, wherein the HVT vector comprises a nucleic acid sequence of at least 95%, or 100% identical to the sequence of a ND Fusion gene SEQ ID NO:13.
